(19) United States
(12) Patent Application Publication
(10) Pub. No.: US 2007/0092882 A1
(43) Pub. Date: Apr. 26, 2007

(54) Analysis of microRNA

(76) Inventors: Hui Wang, Palo Alto, CA (US); Bo U. Curry, Redwood City, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/256,229

(22) Filed: Oct. 21, 2005

(51) Int. Cl. C12Q 1/68; C07H 21/02; C12M 1/34

(52) U.S. Cl. 435/6; 536/23.1; 536/24.3; 435/287.2; 977/924

(57) ABSTRACT

Methods are described in which a sample containing miRNA is contacted with an array having a probe set, followed by interrogating the array to assess binding to the probe set. Probes, probe sets, arrays comprising a probe set, and kits incorporating the probe sets are also described.

Related subject matter is disclosed in copending U.S. patent application Ser. No. 11/173693 filed on Jul. 1, 2005 by Wang entitled “Nucleic Acid Probes for Analysis of Small RNAs and Other Polynucleotides.” Related subject matter is disclosed in copending U.S. patent application Ser. No. 11/048225 filed on Jan. 31, 2005 by Wang entitled “RNA Labeling Method.”

### DESCRIPTION

1. Field of the Invention:

The invention relates generally to methods of biochemical analysis. More specifically, the invention relates to analysis of microRNA.

2. Background of the Invention:

Since the discovery of the biological activity of short interfering RNAs (siRNAs) over a decade ago, so called “small RNAs” (i.e., short non-coding regulatory RNAs that have a defined sequence) have become a subject of intense interest in the research community. See Novina et al., Nature 430: 161-164 (2004). Exemplary short RNAs include siRNAs, microRNAs (miRNAs), tiny non-coding RNAs (tncRNAs) and small modulatory RNAs (smRNAs), as well as many others.

Although the exact biological functions of most small RNAs remain a mystery, it is clear that they are abundant in plants and animals, with up to tens of thousands of copies per cell. For example, to date, over 78 Drosophila microRNA species and 300 human microRNA species have been identified. The levels of the individual species of small RNA, in particular microRNA species, appears to vary according to the developmental stage and type of tissue being examined. It is thought that the levels of particular small RNAs may be correlated with particular phenotypes, as well as with the levels of particular mRNAs and proteins. Further, viral microRNAs have been identified, and their presence has been linked to viral latency (see Pfeffer et al., Science, 304: 734-736 (2004) ).

The sequences of several hundred miRNAs from a variety of different species, including humans, may be found at the microRNA registry (Griffiths-Jones, Nucl. Acids Res. 2004 32:D109-D111), as found at the world-wide website of the Sanger Institute (Cambridge, UK) (which may be accessed by typing “www” followed by “.sanger.ac.uk/cgi-bin/Rfam/mima/browse.pl” into the address bar of a typical internet browser). The sequences of all of the microRNAs deposited at the microRNA registry, including more than 300 microRNA sequences from humans (see Lagos-Quintana et al, Science 294:853-858(2001); Grad et al, Mol Cell 11:1253-1263(2003); Mourelatos et al, Genes Dev 16:720-728(2002); Lagos-Quintana et al, Curr Biol 12:735-739(2002); Lagos-Quintana et al, RNA 9:175-179(2003); Dostie et al, RNA 9:180-186(2003); Lim et al, Science 299:1540(2003); Houbaviy et al, Dev Cell 5:351-358(2003); Michael et al, Mol Cancer Res 1:882-891(2003); Kim et al, Proc Natl Acad Sci U S A 101:360-365(2004); Suh et al, Dev Biol 270:488-498(2004); Kasashima et al, Biochem Biophys Res Commun 322:403-410(2004); and xie et al, Nature 434:338-345(2005)), are incorporated herein by reference. MicroRNAs (miRNAs) are a class of single stranded RNAs of approximately 19-25 nt (nucleotides) in length.

Thus, analysis of of miRNA may be of great importance, for example as a research or diagnostic tool. Analytic methods employing polynucleotide arrays have been used for investigating small RNAs, e.g. miRNAs have become a subject of investigation with microarray analysis. See, e.g., Liu et al., Proc. Nat'l Acad. Sci. USA, 101: 9740-9744 (2004); Thomson et al., Nature Methods, 1:1-7 (2004); and Babak et al., RNA, 10:1813-1819 (2004). A considerable amount of effort is currently being put into developing array platforms to facilitate the analysis of small RNAs, particularly microRNAs. Polynucleotide arrays (such as DNA or RNA arrays) typically include regions of usually different sequence polynucleotides (“capture agents”) arranged in a predetermined configuration on a support. The arrays are “addressable” in that these regions (sometimes referenced as “array features”) have different predetermined locations (“addresses”) on the support of array. The polynucleotide arrays typically are fabricated on planar supports either by depositing previously obtained polynucleotides onto the support in a site specific fashion or by site specific in situ synthesis of the polynucleotides upon the support. After depositing the polynucleotide capture agents onto the support, the support is typically processed (e.g., washed and blocked for example) and stored prior to use.

In use, an array is contacted with a sample or labeled sample containing analytes (typically, but not necessarily, other polynucleotides) under conditions that promote specific binding of the analytes in the sample to one or more of the capture agents present on the array. Thus, the arrays, when exposed to a sample, will undergo a binding reaction with the sample and exhibit an observed binding pattern. This binding pattern can be detected upon interrogating the array. For example all target polynucleotides (for example, DNA) in the sample can be labeled with a suitable label (such as a fluorescent compound), and the label then can be accurately observed (such as by observing the fluorescence pattern) on the array after exposure of the array to the sample. Assuming that the different sequence polynucleotides were correctly deposited in accordance with the predetermined configuration, then the observed binding pattern will be indicative of the presence and/or concentration of one or more components of the sample. Techniques for scanning arrays are described, for example, in U.S. Pat. No. 5,763,870 and U.S. Pat. No. 5,945,679. Still other techniques useful for observing an array are described in U.S. Pat. No. 5,721,435.

There is a continuing need for new methods of analyzing microRNA. The presently described invention addresses this need, and others.

### SUMMARY OF THE INVENTION

The invention thus relates to novel probe sets, arrays, and methods for analyzing microRNA in a sample. In certain embodiments, subject probe sets include a plurality of probes, each probe including a target-complementary sequence independently selected from the group consisting of SEQ ID NOS: 1-1240. In some embodiments, an array comprising a subject probe set is provided. In particular embodiments of a method for analyzing microRNA in a sample, the sample is contacted with an array comprising a probe set that includes at least five probes. Each of the at least five probes includes a target-complementary sequence independently selected from the group consisting of SEQ ID NOS:1-1240. The array is then interrogated to obtain information about miRNAs in the sample.

The invention finds use in a wide variety of diagnostic and research applications. Additional uses and novel features of this invention shall be set forth in part in the descriptions and examples that follow and in part will become apparent to those skilled in the art upon examination of the following specifications or may be learned by the practice of the invention. Practice of the invention may be realized and attained by means of the instruments, combinations, compositions and methods set forth in the specification and particularly pointed out in the appended claims.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein

FIG. 1 schematically illustrates a probe of the invention.

FIGS. 2A-2C schematically illustrate exemplary probes of the invention.

FIG. 3 schematically illustrate an embodiment of the invention.

FIGS. 4A-4C schematically illustrate exemplary methods of the invention.

To facilitate understanding, identical reference numerals have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different order where this is logically possible. However, the order described below is preferred.

It must be noted that, as used in the specification and the appended claims, the singular forms “a,” “an” and “the” include plural referents unless the context clearly dictates otherwise. Thus, for example, reference to “an insoluble support” includes a plurality of insoluble supports. Similarly, reference to “a microRNA” includes a plurality of different identity (sequence) microRNA species.

Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. In this specification and in the claims that follow, reference will be made to a number of terms that shall be defined to have the following meanings unless a contrary intention is apparent. “Optional” or “optionally” means that the subsequently described circumstance may or may not occur, so that the description includes instances where the circumstance occurs and instances where it does not. For example, if a step of a process is optional, it means that the step may or may not be performed, and, thus, the description includes embodiments wherein the step is performed and embodiments wherein the step is not performed (i.e. it is omitted). If an element of an apparatus or composition is optional, the element may or may not be present, and, thus, the description includes embodiments wherein the element is present and embodiments wherein the element is not present (i.e. it is omitted).

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

“Moiety” and “group” are used to refer to a portion of a molecule, typically having a particular functional or structural feature, e.g. a linking group (a portion of a molecule connecting two other portions of the molecule), or an ethyl moiety (a portion of a molecule with a structure closely related to ethane). A moiety is generally bound to one or more other moieties to provide a molecular entity. As a simple example, a hydroxyl moiety bound to an ethyl moiety provides an ethanol molecule. At various points herein, the text may refer to a moiety by the name of the most closely related structure (e.g. an oligonucleotide moiety may be referenced as an oligonucleotide, a mononucleotide moiety may be referenced as a mononucleotide). However, despite this seeming informality of terminology, the appropriate meaning will be clear to those of ordinary skill in the art given the context, e.g. if the referenced term has a portion of its structure replaced with another group, then the referenced term is usually understood to be the moiety. For example, a mononucleotide moiety is a single nucleotide which has a portion of its structure (e.g. a hydrogen atom, hydroxyl group, or other group) replaced by a different moiety (e.g. a linking group, an observable label moiety, or other group). Similarly, an oligonucleotide moiety is an oligonucleotide which has a portion of its structure (e.g. a hydrogen atom, hydroxyl group, or other group) replaced by a different moiety (e.g. a linking group, an observable label moiety, or other group).

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. An “oligonucleotide” is a molecule containing from 2 to about 100 nucleotide subunits. As used herein in the context of a polynucleotide sequence, the term “bases” (or “base”) is synonymous with “nucleotides” (or “nucleotide”), i.e. the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural (not usually occurring in nature) nucleotides, unnatural nucleotide mimetics such as 2′-modified nucleosides, peptide nucleic acids, oligomeric nucleoside phosphonates, and any polynucleotide that has added substituent groups, such as protecting groups or linking moieties.

“Sequence” may refer to a particular sequence of bases and/or may refer to a polynucleotide having the particular sequence of bases and/or may refer to a sub-sequence, i.e. a sequence that is part of a longer sequence. Thus a sequence may be information or may refer to a molecular entity or a portion of a molecular entity, as indicated by the context of the usage.

“Bound” may be used herein to indicate direct or indirect attachment. In the context of chemical structures, “bound” (or “bonded”) may refer to the existence of a chemical bond directly joining two moieties or indirectly joining two moieties (e.g. via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and also embodiments where the attachment is indirect.

“Isolated” or “purified” generally refers to isolation of a substance (compound, polynucleotide, protein, polypeptide, polypeptide, chromosome, etc.) such that the substance comprises a substantial portion of the sample in which it resides (excluding solvents), i.e. greater than the substance is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample (excluding solvents). For example, a sample of isolated RNA will typically comprise at least about 1% total RNA, where percent is calculated in this context as mass (e.g. in micrograms) of total RNA in the sample divided by mass (e.g. in micrograms) of the sum of (total RNA+other constituents in the sample (excluding solvent) ). Techniques for purifying polynucleotides and polypeptides of interest are well known in the art and include, for example, gel electrophresis, ion-exchange chromatography, affinity chromatography, flow sorting, and sedimentation according to density. Generally, a substance is purified when it exists in a sample in an amount, relative to other components of the sample, that is not found naturally. In typical embodiments, the sample includes isolated RNA, such as isolated microRNA, prior to use in the present methods.

The term “sample” as used herein relates to a material or mixture of materials, typically, although not necessarily, in fluid form, containing one or more components of interest. The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of surface-bound oligonucleotides, as is commonly known in the art and described below, is not a mixture of surface-bound oligonucleotides because the species of surface-bound oligonucleotides are spatially distinct and the array is addressable.

The term “analyte” is used herein to refer to a known or unknown component of a sample. In certain embodiments of the invention, an analyte may specifically bind to a capture agent on a support surface if the analyte and the capture agent are members of a specific binding pair. In general, analytes are typically RNA or other polynucleotides. Typically, an “analyte” is referenced as a species in a mobile phase (e.g., fluid), to be detected by a “capture agent” which, in some embodiments, is bound to a support, or in other embodiments, is in solution. However, either of the “analyte” or “capture agent” may be the one which is to be evaluated by the other (thus, either one could be an unknown mixture of components of a sample, e.g., polynucleotides, to be evaluated by binding with the other). A “target” references an analyte.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides, or fragments of double stranded or single-stranded DNA or RNA may employed. Capture agents usually are characterized as being capable of exhibiting “specific binding” for one or more analytes.

The term “specific binding” refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the binding constant of a capture agent and analyte is greater than 10⁶ M⁻¹, greater than 10⁷ M⁻¹, greater than 10⁶ M⁻¹, greater than 10⁹ M⁻¹, greater than 10¹⁰ M⁻¹, usually up to about 10¹² M⁻¹, or even up to about 10¹⁵M⁻¹.

In typical embodiments herein, a “probe” is a capture agent that is directed to a microRNA. An miRNA that a probe is directed to is referenced herein as a “target miRNA”. Each probe of the probe set has a respective target miRNA. A probe set consists of its member probes. A probe/target duplex is a structure formed by hybridizing a probe to its target.

The term “pre-determined” refers to an element whose identity is known prior to its use. For example, a “pre-determined analyte” is an analyte whose identity is known prior to any binding to a capture agent. An element may be known by name, sequence, molecular weight, its function, or any other attribute or identifier. In some embodiments, the term “analyte of interest”, i.e., a known analyte that is of interest, is used synonymously with the term “pre-determined analyte”.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. The term stringent assay conditions refers to the combination of hybridization and wash conditions.

A “stringent hybridization” and “stringent hybridization wash conditions” in the context of nucleic acid hybridization (e.g., as in array, Southern or Northern hybridizations) are sequence dependent, and are different under different experimental conditions. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.1×SSC and 0.1% SDS at 37° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHP04, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Hybridization buffers suitable for use in the methods described herein are well known in the art and may contain salt, buffer, detergent, chelating agents and other components at pre-determined concentrations.

In certain embodiments, the stringency of the wash conditions may affect the degree to which nucleic acids are specifically hybridized to complementary capture agents. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCI at 72° C. for about 15 minutes; or, a salt concentration of about 0.2×SSC at a temperature of at least about 50° C. or about 55° C. to about 60° C. for about 1 to about 20 minutes; or, multiple washes with a solution with a salt concentration of about 0.1×SSC containing 0.1% SDS at 20 to 50° C. for 1 to 15 minutes; or, equivalent conditions. Stringent conditions for washing can also be, e.g., 0.2×SSC/0.1% SDS at 42° C. In instances wherein the nucleic acid molecules are deoxyoligonucleotides (i.e., oligonucleotides), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See, e.g., Ausubel, et al., Short Protocols in Molecular Biology, 3rd ed., Wiley & Sons (1995); Sambrook et al., Molecular Cloning: A Laboratory Manual, Third Edition, Cold Spring Harbor, N.Y. (2001); or Tijssen, Hybridization with Nucleic Acid Probes, Parts I and II (Elsevier, Amsterdam 1993), for detailed descriptions of equivalent hybridization and wash conditions and for reagents and buffers, e.g., SSC buffers and equivalent reagents and conditions.

Those of ordinary skill will readily recognize that alternative but comparable hybridization and wash conditions can be utilized to provide conditions of similar stringency. A specific example of stringent assay conditions is rotating hybridization at a temperature of about 55° C. to about 70° C. in a salt based hybridization buffer with a total monovalent cation concentration of 1.5M (e.g., as described in U.S. patent application Ser. No. 09/655,482 filed on Sep. 5, 2000, the disclosure of which is herein incorporated by reference) followed by washes of 0.5×SSC and 0.1×SSC at room temperature and 37° C.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-i DNA or with random sequence synthetic oligonucleotides (e.g. 25-mers), or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “array” and the equivalent term “microarray” each reference an ordered array of capture agents for binding to aqueous analytes and the like. An “array” includes any two-dimensional or substantially two-dimensional (as well as a three-dimensional) arrangement of spatially addressable regions (i.e., “features”) containing capture agents, particularly polynucleotides, and the like. Any given support may carry one, two, four or more arrays disposed on a surface of a support. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 100 cm², 20 cm² or even less than 10 cm², e.g., less than about 5cm², including less than about 1 cm², less than about 1 mm², e.g., 100 μm², or even smaller. For example, features may have widths (that is, diameter, for a round spot) in the range from a 10 μm² to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter- feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Arrays can be fabricated by depositing (e.g., by contact- or jet-based methods) either precursor units (such as nucleotide or amino acid monomers) or pre-synthesized capture agent. An array is “addressable” when it has multiple regions of different moieties (e.g., different capture agents) such that a region (i.e., a “feature” or “spot” of the array) at a particular predetermined location (i.e., an “address”) on the array will detect a particular target. An “array layout” refers to one or more characteristics of the features, such as feature positioning on the support, one or more feature dimensions, and an indication of a moiety at a given location. “Interrogating” the array refers to obtaining information from the array, especially information about analytes binding to the array. “Hybridization assay” references a process of contacting an array with a mobile phase containing analyte. An “array support” refers to an article that supports an addressable collection of capture agents.

“Linker” references an oligonucleotide moiety that is part of a probe, wherein the linker is bound to the target-complementary sequence. In embodiments in which the probe is bound to a surface of an array support, the target-complementary sequence is bound to the array support via the linker sequence. The linker sequence may be any sequence that does not substantially interfere with hybridization of targets to probes, e.g. the sequence of the probe should be selected to not be complementary to any analytes expected to be assayed. An example used herein is a (T)₁₀ linker (ten contiguous Ts), wherein one end of the (T)₁₀ linker is bound to the target-complementary sequence, and the probe is bound to the array sequence via the other end of the (T)₁₀ linker.

In certain embodiments, a probe includes a Tm enhancement domain that increases the stability of the probe/target duplex. Such “Tm enhancement domains” are described in U.S. patent application Ser. No. 11/173,693, filed by Wang on Jul. 1, 2005, and entitled “Nucleic Acid Probes for Analysis of Small RNAs and Other Polynucleotides”. The Tm enhancement domain may contain a nucleotide clamp and/or a hairpin structure, for example. Briefly, a nucleotide clamp contains a contiguous sequence of up to about 5 nucleotides (i.e., 1, 2, 3, 4 or 5 nucleotides). The identity of the nucleotides employed in the nucleotide clamp may be the same as each other or different to each other. In particular embodiments, the addition of the nucleotide clamp increases the stability of the probe/target duplex, as compared to a probe/target duplex formed in the absence of the clamp. Briefly, a hairpin structure has a loop of at least 3 or 4 nucleotides and a double-stranded stem in which complementary nucleotides bind to each other in an anti-parallel manner. In a duplex formed between a probe containing a hairpin structure and a target, the hairpin region promotes a phenomenon termed stacking (which phenomenon may also be called coaxial stacking) which allows the target to bind more tightly, i.e., more stably, to the probe, as compared to a probe/target duplex formed in the absence of the hairpin structure.

“Complementary” references a property of specific binding between polynucleotides based on the sequences of the polynucleotides. As used herein, a first polynucleotide and a second polynucleotide are complementary if they bind to each other in a hybridization assay under stringent conditions, e.g. if they produce a given or detectable level of signal in a hybridization assay. Portions of polynucleotides are complementary to each other if they follow conventional base-pairing rules, e.g. A pairs with T (or U) and G pairs with C, although small regions (e.g. less than about 5 bases) of mismatch, insertion, or deleted sequence may be present. In this regard, “strictly complementary” is a term used to characterize a first polynucleotide and a second polynucleotide, such as a target and a probe directed to the target, and means that every base in a sequence (or sub-sequence) of contiguous bases in the first polynucleotide has a corresponding complementary base in a corresponding sequence (or sub-sequence) of contiguous bases in the second polynucleotide. “Strictly complementary” means that there are no insertions,deletions, or substitutions in either of the first and second polynucleotides with respect to the other polynucleotide (over the complementary region). Put another way, every base of the complementary region may be paired with its complementary base, i.e. following normal base-pairing rules. The region that is complementary (the complementary region) between a first polynucleotide and a second polynucleotide (e.g. a target analyte and a capture agent) is typically at least about 10 bases long, more typically at least about 12 bases long, still more typically at least about 15 bases long, or at least about 17 bases long. In various typical embodiments, the region that is complementary between a first polynucleotide and a second polynucleotide (e.g. target and a capture agent) may be up to about 30 bases long, or longer, or up to about 27 bases long, up to about 25 bases long, or up to about 23 bases long.

A “target-complementary sequence” references a polynucleotide sequence that is complementary to a target. In the context of a probe which comprises a target-complementary sequence, wherein the probe is directed to a target miRNA, the target-complementary sequence generally is a contiguous nucleotide sequence that is complementary to the nucleotide sequence of the target miRNA and is of a length that is sufficient to provide specific binding between the probe and the target miRNA. In particular embodiments, the target-complementary sequence is complementary to at least 10 bases, at least 12 bases, at least 15 bases, at least 17 bases of the target mIRNA, and may be complementary to up to the full length of the target miRNA.

As used herein, “fully-complementary” is a term used to characterize a probe; a “fully-complementary” probe comprises a target-complementary sequence such that the target-complementary sequence is strictly complementary to the full sequence of the target miRNA to which the probe is directed. Therefore, in a probe that is fully-complementary to its target miRNA, every base of the complete target miRNA sequence has a corresponding complementary base in the target-complementary sequence, such that the target-complementary sequence may be aligned base-for-base along the full sequence of a target miRNA to highlight the complementarity. And, under conditions sufficient to allow for hybridization to occur, a fully-complementary probe may hybridize to its target miRNA such that the entire target sequence can be hybridized to the target-complementary sequence, base to base, following normal hybridization rules (e.g. A base pairs with T (or U), and G base pairs with C). The probe that is fully-complementary to its target miRNA may have additional sequences, e.g. at the 5′- and/or 3′-end of the target-complementary sequence, i.e. the fully complementary sequence may be a portion of a longer sequence.

As used herein, “not fully-complementary” is a term used to characterize a probe; a probe that is “not fully-complementary” lacks a sequence of contiguous bases that is strictly complementary to the full sequence of the target miRNA to which the probe is directed. It should be noted that, under conditions sufficient to allow for hybridization to occur, a not fully-complementary probe may hybridize to its target miRNA such that the target-complementary sequence of the probe can be hybridized to a portion of the target miRNA sequence, base to base, following normal hybridization rules (e.g. A base pairs with T (or U), and G base pairs with C). In such a case, the target-complementary sequence that is complementary to the portion of the target miRNA sequence is typically at least one, more typically at least two, still more typically at least 3, at least 4, at least 5 bases shorter than the full sequence of the target miRNA, and may be up to about 10 bases, or possibly up to about 15 bases, shorter than the full sequence of the target miRNA.

If a polynucleotide, e.g. a probe, is “directed to” a target, the polynucleotide has a sequence that is complementary to a sequence in that target and will specifically bind (i.e. hybridize) to that target under hybridization conditions. The hybridization conditions typically are selected to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. Such hybridization conditions are typically known in the art. Examples of such appropriate hybridization conditions are also disclosed herein for hybridization of a sample to a microarray. The target will typically be an miRNA for embodiments discussed herein.

Accordingly, the invention provides novel probe sets, arrays, and methods for analyzing microRNA in a sample. In certain embodiments, subject probe sets include a plurality of probes, each probe including a target-complementary sequence independently selected from the group consisting of SEQ ID NOS:1-1240. In some embodiments, an array comprising a subject probe set is provided. In particular embodiments of a method for analyzing microRNA in a sample, the sample is contacted with an array comprising a probe set that includes at least five probes. Each of the at least five probes includes a target-complementary sequence independently selected from the group consisting of SEQ ID NOS:1- 1240. The array is then interrogated to obtain information about miRNAs in the sample.

In further describing the present invention, subject probes and probe sets for detecting target miRNAs will be described first, followed by arrays that include such probes. This is followed by a description of how the subject probes may be used to assess polynucleotides (e.g. microRNAs) in a sample. Finally, representative kits for use in practicing the subject methods will be discussed.

Probes

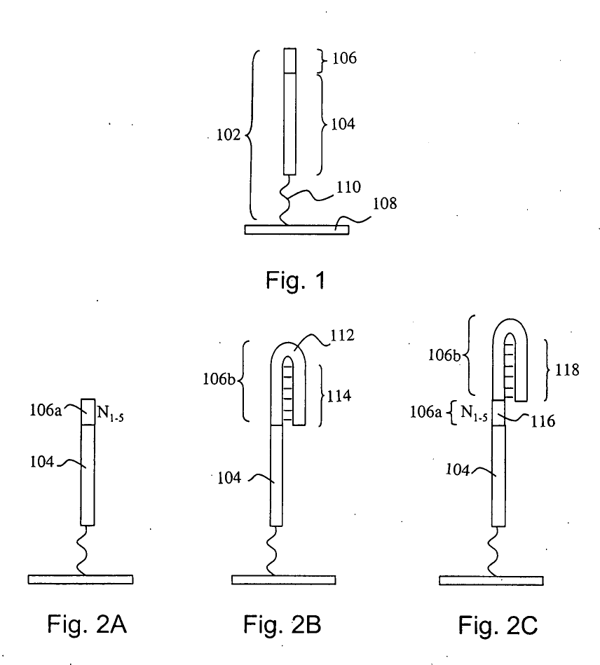

As mentioned above and with reference to FIG. 1, the invention provides a probe 102 for detecting a microRNA. The probe typically includes a first region 104 (i.e., a “target-complementary sequence”) directed to a target miRNA. In particular embodiments the probe further includes an optional Tm enhancement domain 106. In certain embodiments, the probe 102 further includes a linker 110, and the probe is attached to a solid support 108. As will be described in greater detail below, the solid support may be part of an array, and the array may contain a plurality of different probes for detecting a plurality of target miRNAs. The configuration shown in the figure is typical, with the Tm enhancement domain 106 being attached immediately adjacent to the target-complementary sequence 104, which is attached immediately adjacent to the linker 110, which is attached to solid support 108.

As mentioned above, the probes of the invention (subject probes) may be used to detect microRNAs (miRNAs). The methods and compositions described herein may be used to detect any microRNA for which a sequence has been deposited at the microRNA registry, as well as others. (See Griffiths-Jones, Nucl. Acids Res. 2004 32:D109-D111; as well as the world-wide website of the Sanger Institute, supra.) The microRNA registry includes the sequences which are given SEQ ID NOS: 1268-1581 herein. As will be described in greater detail below, the target miRNA to be detected generally has a 3′ end or 5′ end (depending on which end of the probe for detecting that target miRNA is attached to the solid support) of known sequence.

A subject probe may be in the range of about 10 to about 100 bases in length. In certain embodiments, however, a subject probe may be about 18 to about 70 bases, about 19 to about 60 bases, or about 20 to about 50 bases in length. Target-complementary sequence 104 generally contains a contiguous nucleotide sequence that is complementary to the nucleotide sequence of a corresponding miRNA and is of a length that is sufficient to provide specific binding between the probe and the corresponding miRNA. Since miRNAs are generally in the range of about 19 to about 25 nucleotides (nt) in length, target-complementary sequence 104 is generally at least about 10 nt, at least about 12 nt, or at least about 15 nt in length. In certain embodiments target-complementary sequence 104 may be as long as about 18 nt, as long as about 20 nt, as long as about 22 nt, or as long as about 25 nt in length, or longer. The probe 102, if it is attached to a solid support 108, may be attached via its 3′ end or its 5′ end. If the probe 102 is attached to a solid support 108 via its 3′ end, the nucleotide at the 5′ end of the target-complementary sequence 104 of the probe 102 generally base pairs with the 3′ terminal nucleotide of a target miRNA to be detected. Conversely, if the probe 102 is attached to a solid support via its 5′ end, the nucleotide at the 3′ end of the target-complementary sequence 104 of the probe 102 generally base pairs with the 5′ terminal nucleotide of a target miRNA to be detected. A subject probe need not be complementary to the entire length of a corresponding target miRNA to provide for assessing the miRNA in a sample, and a target miRNA to be detected need not be complementary to the entire length of a subject probe.

The target-complementary sequence 104 therefore is directed to, i.e., hybridizes to and may be used to detect, a particular target miRNA. In many embodiments, the target-complementary sequence 104 is specific for a particular target miRNA, in that it can detect a particular target miRNA, even in the presence of other RNAs, e.g., other miRNAs, further e.g. RNA or isolated “small RNA” (isolated RNA smaller than about 500 bases in length, e.g. smaller than about 300 bases in length). In other words, a subject probe contains a target-complementary sequence 104 that is complementary to a particular target miRNA.

Table 1 includes a listing of sequences for some possible target-complementary sequences to be used in probes in accordance with the present invention. The table includes the SEQ ID NO: for each sequence, the base listing of the sequence, the name of the target miRNA, as well as the calculated melting temperature of a duplex between the probe and the target miRNA that the probe is directed to. Note that each probe included a single base nucleotide clamp (a G) at the 5′ end of the target-complementary sequences, which was accounted for in calculating the calculated melting temperature.

TABLE 1

| | | | |
|---|---|---|---|
| SEQ | | | |
| ID | | Target | Tm |
| NO: | Sequence | miRNA | (calc) |

| | | | |
|---|---|---|---|
| 1 | GCACTGATTTCGAATGGT | dme-miR-285 | 58.8 |
| 2 | GCTCCTCAAAGCTGG | dme-miR-2b | 55.4 |
| 3 | TGAGACACACTTTGCCC | dme-miR-3 | 58.8 |
| 4 | CTCACAGTATAATCCTGTGATT | dme-miR-308 | 59.4 |
| 5 | TCAGCTATGCCGACATCT | dme-miR-31a | 59.1 |
| 6 | AAAAAGAACAGCCACTGTG | dme-miR-6 | 58.7 |
| 7 | AACTATACAACCTACTACC | hsa-let-7a | 51.7 |
| 8 | AACTATACAACCTACTACCT | hsa-let-7a | 53.7 |
| 9 | AACCACACAACCTACTA | hsa-let-7b | 52.8 |
| 10 | AACCACACAACCTACTAC | hsa-let-7b | 55.4 |
| 11 | AACCATACAACCTACTAC | hsa-let-7c | 51.6 |
| 12 | AACCATACAACCTACTACC | hsa-let-7c | 55.5 |
| 13 | AACCATACAACCTACTACCT | hsa-let-7c | 57.3 |
| 14 | ACTATGCAACCTACTACCT | hsa-let-7d | 55.8 |
| 15 | ACTATACAACCTCCTACCTC | hsa-let-7e | 57.6 |
| 16 | ACTATACAACCTCCTACCTCA | hsa-let-7e | 59.9 |
| 17 | AACTATACAATCTACTACCT | hsa-let-7f | 50.8 |
| 18 | AACTATACAATCTACTACCTC | hsa-let-7f | 53.3 |
| 19 | AACTATACAATCTACTACCTCA | hsa-let-7f | 55.5 |
| 20 | ACTGTACAAACTACTACCT | hsa-let-7g | 53.6 |
| 21 | ACTGTACAAACTACTACCTC | hsa-let-7g | 56 |
| 22 | ACTGTACAAACTACTACCTCA | hsa-let-7g | 58.2 |
| 23 | ACAGCACAAACTACTACC | hsa-let-7i | 55.3 |
| 24 | ACAGCACAAACTACTACCT | hsa-let-7i | 57.2 |
| 25 | ACAGCACAAACTACTACCTC | hsa-let-7i | 59.4 |
| 26 | TACATACTTCTTTACATTCC | hsa-miR-1 | 52.1 |
| 27 | TACATACTTCTTTACATTCCA | hsa-miR-1 | 54.4 |
| 28 | CACAAGTTCGGATCTAC | hsa-miR-100 | 55.2 |
| 29 | CACAAGTTCGGATCTACG | hsa-miR-100 | 59.2 |
| 30 | CTTCAGTTATCACAGTACTGTA | hsa-miR-101 | 59.1 |
| 31 | TCATAGCCCTGTACAAT | hsa-miR-103 | 51.9 |
| 32 | TCATAGCCCTGTACAATG | hsa-miR-103 | 54.7 |
| 33 | ACAGGAGTCTGAGCA | hsa-miR-105 | 52.5 |
| 34 | ACAGGAGTCTGAGCAT | hsa-miR-105 | 53.6 |
| 35 | ACAGGAGTCTGAGCATT | hsa-miR-105 | 55.4 |
| 36 | ACAGGAGTCTGAGCATTT | hsa-miR-105 | 56.9 |
| 37 | ACAGGAGTCTGAGCATTTG | hsa-miR-105 | 59.3 |
| 38 | GCTACCTGCACTGTAAG | hsa-miR-106a | 56.3 |
| 39 | ATCTGCACTGTCAGCA | hsa-miR-106b | 54.9 |
| 40 | TGATAGCCCTGTACAATGC | hsa-miR-107 | 58.8 |
| 41 | AATGCCCCTAAAAATCC | hsa-miR-108 | 53.2 |
| 42 | AATGCCCCTAAAAATCCT | hsa-miR-108 | 55.3 |
| 43 | AATGCCCCTAAAAATCCTT | hsa-miR-108 | 56.8 |
| 44 | AATGCCCCTAAAAATCCTTA | hsa-miR-108 | 57.2 |
| 45 | AATGCCCCTAAAAATCCTTAT | hsa-miR-108 | 57.8 |
| 46 | CACAAATTCGGATCTACAG | hsa-miR-10a | 57.4 |
| 47 | CACAAATTCGGATCTACAGG | hsa-miR-10a | 60.8 |
| 48 | CACAAATTCGGATCTACAGGG | hsa-miR-10a | 63.9 |
| 49 | ACAAATTCGGTTCTACAGG | hsa-miR-10b | 57.7 |
| 50 | ACAAACACCATTGTCA | hsa-miR-122a | 51.3 |
| 51 | ACAAACACCATTGTCAC | hsa-miR-122a | 54.1 |
| 52 | ACAAACACCATTGTCACA | hsa-miR-122a | 56.6 |
| 53 | ACAAACACCATTGTCACAC | hsa-miR-122a | 58.8 |
| 54 | TGGCATTCACCGC | hsa-miR-124a | 51.5 |
| 55 | TGGCATTCACCGCG | hsa-miR-124a | 56.6 |
| 56 | TGGCATTCACCGCGT | hsa-miR-124a | 59.3 |
| 57 | CACAGGTTAAAGGGTCTC | hsa-miR-125a | 58.9 |
| 58 | CACAGGTTAAAGGGTCTCA | hsa-miR-125a | 61.2 |
| 59 | CACAGGTTAAAGGGTCTCAG | hsa-miR-125a | 62.8 |
| 60 | TCACAAGTTAGGGTCTC | hsa-miR-125b | 54.2 |
| 61 | TCACAAGTTAGGGTCTCA | hsa-miR-125b | 56.8 |
| 62 | GCATTATTACTCACGGTAC | hsa-miR-126 | 56.3 |
| 63 | GCATTATTACTCACGGTACG | hsa-miR-126 | 60 |
| 64 | CGCGTACCAAAAGTAATAATG | hsa-miR-126* | 59.7 |
| 65 | AGCCAAGCTCAGACGG | hsa-miR-127 | 59.6 |
| 66 | AAAAGAGACCGGTTC | hsa-miR-128a | 50.8 |
| 67 | AAAAGAGACCGGTTCA | hsa-miR-128a | 53.8 |
| 68 | AAAAGAGACCGGTTCAC | hsa-miR-128a | 56.5 |
| 69 | AAAAGAGACCGGTTCACT | hsa-miR-128a | 58.3 |
| 70 | GAAAGAGACCGGTTCAC | hsa-miR-128b | 58.8 |
| 71 | GAAAGAGACCGGTTCACT | hsa-miR-128b | 60.7 |
| 72 | GAAAGAGACCGGTTCACTG | hsa-miR-128b | 62.9 |
| 73 | GCAAGCCCAGACC | hsa-miR-129 | 53.3 |
| 74 | GCAAGCCCAGACCG | hsa-miR-129 | 58.6 |
| 75 | ATGCCCTTTTAACATTGC | hsa-miR-130a | 55.1 |
| 76 | ATGCCCTTTTAACATTGCA | hsa-miR-130a | 57.5 |
| 77 | ATGCCCTTTCATCATTG | hsa-miR-130b | 52.9 |
| 78 | CGACCATGGCTGTAG | hsa-miR-132 | 55.2 |
| 79 | CGACCATGGCTGTAGA | hsa-miR-132 | 57.9 |
| 80 | ACAGCTGGTTGAAGGG | hsa-miR-133a | 57 |
| 81 | ACAGCTGGTTGAAGGGG | hsa-miR-133a | 61 |
| 82 | ACAGCTGGTTGAAGGGGA | hsa-miR-133a | 63.3 |
| 83 | TAGCTGGTTGAAGGGGA | hsa-miR-133b | 58.9 |
| 84 | TAGCTGGTTGAAGGGGAC | hsa-miR-133b | 61.2 |
| 85 | TAGCTGGTTGAAGGGGACC | hsa-miR-133b | 64.8 |
| 86 | CCCTCTGGTCAACC | hsa-miR-134 | 54.3 |
| 87 | CCCTCTGGTCAACCA | hsa-miR-134 | 57.4 |
| 88 | TCACATAGGAATAAAAAGCCA | hsa-miR-135a | 58 |
| 89 | TCACATAGGAATAAAAAGCCAT | hsa-miR-135a | 58.5 |
| 90 | TCACATAGGAATAAAAAGCCATA | hsa-miR-135a | 58.8 |
| 91 | CACATAGGAATGAAAAGC | hsa-miR-135b | 54.3 |
| 92 | CACATAGGAATGAAAAGCC | hsa-miR-135b | 57.8 |
| 93 | TCCATCATCAAAACAAATGGA | hsa-miR-136 | 59.3 |
| 94 | CTACGCGTATTCTTAAG | hsa-miR-137 | 51.8 |
| 95 | CTACGCGTATTCTTAAGC | hsa-miR-137 | 56 |
| 96 | CTACGCGTATTCTTAAGCA | hsa-miR-137 | 58.2 |
| 97 | CTACGCGTATTCTTAAGCAA | hsa-miR-137 | 59.4 |
| 98 | CTACGCGTATTCTTAAGCAAT | hsa-miR-137 | 59.9 |
| 99 | GATTCACAACACCAGC | hsa-miR-138 | 54.9 |
| 100 | GATTCACAACACCAGCT | hsa-miR-138 | 57 |
| 101 | AGACACGTGCACTGTAG | hsa-miR-139 | 57.1 |
| 102 | AGACACGTGCACTGTAGA | hsa-miR-139 | 59.5 |
| 103 | CTACCATAGGGTAAAACCA | hsa-miR-140 | 57.5 |
| 104 | CTACCATAGGGTAAAACCAC | hsa-miR-140 | 59.7 |
| 105 | CCATCTTTACCAGACA | hsa-miR-141 | 52 |
| 106 | CCATCTTTACCAGACAG | hsa-miR-141 | 54.3 |
| 107 | CCATCTTTACCAGACAGT | hsa-miR-141 | 56.9 |
| 108 | CCATCTTTACCAGACAGTG | hsa-miR-141 | 59.2 |
| 109 | TCCATAAAGTAGGAAACACTAC | hsa-miR-142-3p | 58.6 |
| 110 | GTAGTGCTTTCTACTTTAT | hsa-miR-142-5p | 51.8 |
| 111 | GTAGTGCTTTCTACTTTATG | hsa-miR-142-5p | 54.3 |
| 112 | TGAGCTACAGTGCTTCATC | hsa-miR-143 | 58.7 |
| 113 | CTAGTACATCATCTATACTGTA | hsa-miR-144 | 54.5 |
| 114 | AAGGGATTCCTGGGAAAA | hsa-miR-145 | 58.6 |
| 115 | AAGGGATTCCTGGGAAAAC | hsa-miR-145 | 60.9 |
| 116 | AAGGGATTCCTGGGAAAACT | hsa-miR-145 | 62.5 |
| 117 | AACCCATGGAATTCAG | hsa-miR-146a | 51.2 |
| 118 | AACCCATGGAATTCAGT | hsa-miR-146a | 54.2 |
| 119 | AACCCATGGAATTCAGTT | hsa-miR-146a | 55.8 |
| 120 | AACCCATGGAATTCAGTTC | hsa-miR-146a | 58.2 |
| 121 | AACCCATGGAATTCAGTTCT | hsa-miR-146a | 59.9 |
| 122 | AGCCTATGGAATTCAGT | hsa-miR-146b | 52.3 |
| 123 | AGCCTATGGAATTCAGTT | hsa-miR-146b | 54.1 |
| 124 | AGCCTATGGAATTCAGTTC | hsa-miR-146b | 56.5 |
| 125 | AGCCTATGGAATTCAGTTCT | hsa-miR-146b | 58.3 |
| 126 | GCAGAAGCATTTCCAC | hsa-miR-147 | 55.1 |
| 127 | GCAGAAGCATTTCCACA | hsa-miR-147 | 57.8 |
| 128 | ACAAAGTTCTGTAGTGCAC | hsa-miR-148a | 57.4 |
| 129 | ACAAAGTTCTGTAGTGCACT | hsa-miR-148a | 59.1 |
| 130 | ACAAAGTTCTGTGATGCAC | hsa-miR-148b | 58.2 |
| 131 | ACAAAGTTCTGTGATGCACT | hsa-miR-148b | 59.8 |
| 132 | GGAGTGAAGACACG | hsa-miR-149 | 51 |
| 133 | GGAGTGAAGACACGG | hsa-miR-149 | 55.7 |
| 134 | GGAGTGAAGACACGGA | hsa-miR-149 | 58.5 |
| 135 | CACTGGTACAAGGG | hsa-miR-150 | 50.3 |
| 136 | CACTGGTACAAGGGT | hsa-miR-150 | 53.6 |
| 137 | CACTGGTACAAGGGTT | hsa-miR-150 | 55.5 |
| 138 | CACTGGTACAAGGGTTG | hsa-miR-150 | 58.1 |
| 139 | CCTCAAGGAGCTTCAG | hsa-miR-151 | 56.2 |
| 140 | CCTCAAGGAGCTTCAGT | hsa-miR-151 | 58.8 |
| 141 | CCCAAGTTCTGTCATGC | hsa-miR-152 | 58.8 |
| 142 | TCACTTTTGTGACTATGC | hsa-miR-153 | 54 |
| 143 | TCACTTTTGTGACTATGCA | hsa-miR-153 | 56.4 |
| 144 | TCACTTTTGTGACTATGCAA | hsa-miR-153 | 57.7 |
| 145 | CGAAGGCAACACG | hsa-miR-154 | 52.3 |
| 146 | CGAAGGCAACACGG | hsa-miR-154 | 56.8 |
| 147 | CGAAGGCAACACGGA | hsa-miR-154 | 59.5 |
| 148 | AATAGGTCAACCGTGT | hsa-miR-154* | 52.5 |
| 149 | AATAGGTCAACCGTGTA | hsa-miR-154* | 53.2 |
| 150 | AATAGGTCAACCGTGTAT | hsa-miR-154* | 54.1 |
| 151 | AATAGGTCAACCGTGTATG | hsa-miR-154* | 56.6 |
| 152 | AATAGGTCAACCGTGTATGA | hsa-miR-154* | 58.8 |
| 153 | AATAGGTCAACCGTGTATGAT | hsa-miR-154* | 59.3 |
| 154 | CCCCTATCACGATTAG | hsa-miR-155 | 52.1 |
| 155 | CCCCTATCACGATTAGC | hsa-miR-155 | 56.8 |
| 156 | CCCCTATCACGATTAGCA | hsa-miR-155 | 59.3 |
| 157 | CCCCTATCACGATTAGCAT | hsa-miR-155 | 59.9 |
| 158 | CACAAACCATTATGTGCTG | hsa-miR-15a | 58.2 |
| 159 | CACAAACCATTATGTGCTGC | hsa-miR-15a | 61.7 |
| 160 | CACAAACCATTATGTGCTGCT | hsa-miR-15a | 63.1 |
| 161 | TGTAAACCATGATGTGC | hsa-miR-15b | 53.1 |
| 162 | TGTAAACCATGATGTGCT | hsa-miR-15b | 55.1 |
| 163 | TGTAAACCATGATGTGCTG | hsa-miR-15b | 57.5 |
| 164 | CGCCAATATTTACGTGC | hsa-miR-16 | 56.8 |
| 165 | CGCCAATATTTACGTGCT | hsa-miR-16 | 58.5 |
| 166 | ACAAGTGCCTTCACTGC | hsa-miR-17-3p | 58.6 |
| 167 | ACTACCTGCACTGTAAGC | hsa-miR-17-5p | 57.5 |
| 168 | ACTACCTGCACTGTAAGCA | hsa-miR-17-5p | 59.8 |
| 169 | ACTCACCGACAGCG | hsa-miR-181a | 54.8 |
| 170 | ACTCACCGACAGCGT | hsa-miR-181a | 57.7 |
| 171 | ACTCACCGACAGCGTT | hsa-miR-181a | 59.2 |
| 172 | CCCACCGACAGCAA | hsa-miR-181b | 57.7 |
| 173 | CCCACCGACAGCAAT | hsa-miR-181b | 58.4 |
| 174 | ACTCACCGACAGGTTGA | hsa-miR-181c | 59.6 |
| 175 | AACCCACCGACAAC | hsa-miR-181d | 52.9 |
| 176 | AACCCACCGACAACA | hsa-miR-181d | 55.9 |
| 177 | AACCCACCGACAACAA | hsa-miR-181d | 57.6 |
| 178 | AACCCACCGACAACAAT | hsa-miR-181d | 58.3 |
| 179 | TGTGAGTTCTACCATTG | hsa-miR-182 | 51.7 |
| 180 | TGTGAGTTCTACCATTGC | hsa-miR-182 | 56.1 |
| 181 | TAGTTGGCAAGTCTAGAACCA | hsa-miR-182* | n/d |
| 182 | TAGTTGGCAAGTCTAGA | hsa-miR-182* | 52 |
| 183 | TAGTTGGCAAGTCTAGAA | hsa-miR-182* | 53.8 |
| 184 | TAGTTGGCAAGTCTAGAAC | hsa-miR-182* | 56.2 |
| 185 | TAGTTGGCAAGTCTAGAACC | hsa-miR-182* | 59.7 |
| 186 | CAGTGAATTCTACCAGT | hsa-miR-183 | 52.9 |
| 187 | CAGTGAATTCTACCAGTG | hsa-miR-183 | 55.6 |
| 188 | CAGTGAATTCTACCAGTGC | hsa-miR-183 | 59.6 |
| 189 | ACCCTTATCAGTTCTCCG | hsa-miR-184 | 57.9 |
| 190 | ACCCTTATCAGTTCTCCGT | hsa-miR-184 | 60.2 |
| 191 | ACCCTTATCAGTTCTCCGTC | hsa-miR-184 | 62.3 |
| 192 | GAACTGCCTTTCTCT | hsa-miR-185 | 50.6 |
| 193 | AAGCCCAAAAGGAGAATTC | hsa-miR-186 | 58.5 |
| 194 | AAGCCCAAAAGGAGAATTCT | hsa-miR-186 | 60.2 |
| 195 | AAGCCCAAAAGGAGAATTCTT | hsa-miR-186 | 61.3 |
| 196 | CGGCTGCAACAC | hsa-miR-187 | 50.3 |
| 197 | CGGCTGCAACACA | hsa-miR-187 | 53.7 |
| 198 | CGGCTGCAACACAA | hsa-miR-187 | 55.6 |
| 199 | CGGCTGCAACACAAG | hsa-miR-187 | 57.7 |
| 200 | ACCCTCCACCATGC | hsa-miR-188 | 54.1 |
| 201 | ACCCTCCACCATGCA | hsa-miR-188 | 57.2 |
| 202 | ACTGATATCAGCTCAGTAGG | hsa-miR-189 | 57.5 |
| 203 | TATCTGCACTAGATGCA | hsa-miR-18a | 51.6 |
| 204 | TATCTGCACTAGATGCAC | hsa-miR-18a | 54.3 |
| 205 | TATCTGCACTAGATGCACC | hsa-miR-18a | 57.9 |
| 206 | TATCTGCACTAGATGCACCT | hsa-miR-18a | 59.7 |
| 207 | TAACTGCACTAGATGCA | hsa-miR-18b | 52.5 |
| 208 | TAACTGCACTAGATGCAC | hsa-miR-18b | 55.1 |
| 209 | TAACTGCACTAGATGCACC | hsa-miR-18b | 58.7 |
| 210 | ACCTAATATATCAAACATATCA | hsa-miR-190 | 51.8 |
| 211 | AGCTGCTTTTGGGA | hsa-miR-191 | 50.3 |
| 212 | AGCTGCTTTTGGGAT | hsa-miR-191 | 51.5 |
| 213 | AGCTGCTTTTGGGATT | hsa-miR-191 | 53.4 |
| 214 | GGGGACGAAATCC | hsa-miR-191* | 50.1 |
| 215 | GGGGACGAAATCCA | hsa-miR-191* | 53.7 |
| 216 | GGGGACGAAATCCAA | hsa-miR-191* | 55.7 |
| 217 | GGGGACGAAATCCAAG | hsa-miR-191* | 57.9 |
| 218 | GGCTGTCAATTCATAG | hsa-miR-192 | 50 |
| 219 | GGCTGTCAATTCATAGG | hsa-miR-192 | 54.4 |
| 220 | GGCTGTCAATTCATAGGT | hsa-miR-192 | 57 |
| 221 | GGCTGTCAATTCATAGGTC | hsa-miR-192 | 59.3 |
| 222 | CTGGGACTTTGTAGG | hsa-miR-193a | 51.7 |
| 223 | CTGGGACTTTGTAGGC | hsa-miR-193a | 56.7 |
| 224 | AAAGCGGGACTTTGA | hsa-miR-193b | 52.9 |
| 225 | AAAGCGGGACTTTGAG | hsa-miR-193b | 55.1 |
| 226 | AAAGCGGGACTTTGAGG | hsa-miR-193b | 59.1 |
| 227 | TCCACATGGAGTTGC | hsa-miR-194 | 53.1 |
| 228 | TCCACATGGAGTTGCT | hsa-miR-194 | 55.3 |
| 229 | GCCAATATTTCTGTGCTG | hsa-miR-195 | 56.5 |
| 230 | CCAACAACATGAAACT | hsa-miR-196a | 51.6 |
| 231 | CCAACAACATGAAACTA | hsa-miR-196a | 52.3 |
| 232 | CCAACAACATGAAACTAC | hsa-miR-196a | 54.9 |
| 233 | CCAACAACATGAAACTACC | hsa-miR-196a | 58.4 |
| 234 | CCAACAACAGGAAAC | hsa-miR-196b | 51.9 |
| 235 | CCAACAACAGGAAACT | hsa-miR-196b | 54.1 |
| 236 | CCAACAACAGGAAACTA | hsa-miR-196b | 54.7 |
| 237 | CCAACAACAGGAAACTAC | hsa-miR-196b | 57.2 |
| 238 | GCTGGGTGGAGAA | hsa-miR-197 | 50.7 |
| 239 | GCTGGGTGGAGAAG | hsa-miR-197 | 53.5 |
| 240 | GCTGGGTGGAGAAGG | hsa-miR-197 | 58.4 |
| 241 | CCTATCTCCCCTCTG | hsa-miR-198 | 52.5 |
| 242 | CCTATCTCCCCTCTGG | hsa-miR-198 | 57.2 |
| 243 | CCTATCTCCCCTCTGGA | hsa-miR-198 | 60 |
| 244 | GAACAGGTAGTCTGAACAC | hsa-miR-199a | 59.2 |
| 245 | AACCAATGTGCAGACTAC | hsa-miR-199a* | 56.1 |
| 246 | AACCAATGTGCAGACTACT | hsa-miR-199a* | 58 |
| 247 | GAACAGATAGTCTAAACACTG | hsa-miR-199b | 57.5 |
| 248 | TCAGTTTTGCATAGATTTGC | hsa-miR-19a | 56.8 |
| 249 | TCAGTTTTGCATAGATTTGCA | hsa-miR-19a | 58.9 |
| 250 | TCAGTTTTGCATGGATTTG | hsa-miR-19b | 56.5 |
| 251 | ACATCGTTACCAGACAG | hsa-miR-200a | 54.4 |
| 252 | ACATCGTTACCAGACAGT | hsa-miR-200a | 56.9 |
| 253 | TCCAGCACTGTCCG | hsa-miR-200a* | 54.4 |
| 254 | TCCAGCACTGTCCGG | hsa-miR-200a* | 58.9 |
| 255 | GTCATCATTACCAGGC | hsa-miR-200b | 53.2 |
| 256 | GTCATCATTACCAGGCA | hsa-miR-200b | 56 |
| 257 | GTCATCATTACCAGGCAG | hsa-miR-200b | 58 |
| 258 | CCATCATTACCCGG | hsa-miR-200c | 51 |
| 259 | CCATCATTACCCGGC | hsa-miR-200c | 56.3 |
| 260 | CCATCATTACCCGGCA | hsa-miR-200c | 59 |
| 261 | TTTTCCCATGCCCT | hsa-miR-202 | 50.5 |
| 262 | TTTTCCCATGCCCTA | hsa-miR-202 | 51.4 |
| 263 | TTTTCCCATGCCCTAT | hsa-miR-202 | 52.5 |
| 264 | TTTTCCCATGCCCTATA | hsa-miR-202 | 53.2 |
| 265 | TTTTCCCATGCCCTATAC | hsa-miR-202 | 55.9 |
| 266 | TTTTCCCATGCCCTATACC | hsa-miR-202 | 59.5 |
| 267 | AAAGAAGTATATGCATAGGA | hsa-miR-202* | 52.4 |
| 268 | AAAGAAGTATATGCATAGGAA | hsa-miR-202* | 53.9 |
| 269 | AAAGAAGTATATGCATAGGAAA | hsa-miR-202* | 55.3 |
| 270 | CTAGTGGTCCTAAACA | hsa-miR-203 | 51.1 |
| 271 | CTAGTGGTCCTAAACAT | hsa-miR-203 | 52.1 |
| 272 | CTAGTGGTCCTAAACATT | hsa-miR-203 | 53.9 |
| 273 | CTAGTGGTCCTAAACATTT | hsa-miR-203 | 55.5 |
| 274 | CTAGTGGTCCTAAACATTTC | hsa-miR-203 | 57.7 |
| 275 | CTAGTGGTCCTAAACATTTCA | hsa-miR-203 | 59.8 |
| 276 | AGGCATAGGATGACAAAGG | hsa-miR-204 | 58.8 |
| 277 | CAGACTCCGGTGG | hsa-miR-205 | 52.6 |
| 278 | CAGACTCCGGTGGA | hsa-miR-205 | 55.9 |
| 279 | CAGACTCCGGTGGAA | hsa-miR-205 | 57.7 |
| 280 | CAGACTCCGGTGGAAT | hsa-miR-205 | 58.5 |
| 281 | CCACACACTTCCTT | hsa-miR-206 | 50.1 |
| 282 | CCACACACTTCCTTA | hsa-miR-206 | 51 |
| 283 | CCACACACTTCCTTAC | hsa-miR-206 | 54 |
| 284 | CCACACACTTCCTTACA | hsa-miR-206 | 56.7 |
| 285 | CCACACACTTCCTTACAT | hsa-miR-206 | 57.5 |
| 286 | CCACACACTTCCTTACATT | hsa-miR-206 | 58.8 |
| 287 | ACAAGCTTTTTGCTCG | hsa-miR-208 | 53.4 |
| 288 | ACAAGCTTTTTGCTCGTC | hsa-miR-208 | 58.2 |
| 289 | ACAAGCTTTTTGCTCGTCT | hsa-miR-208 | 59.9 |
| 290 | CTACCTGCACTATAAGC | hsa-miR-20a | 52.9 |
| 291 | CTACCTGCACTATAAGCA | hsa-miR-20a | 55.6 |
| 292 | CTACCTGCACTATAAGCAC | hsa-miR-20a | 57.9 |
| 293 | CTACCTGCACTATAAGCACT | hsa-miR-20a | 59.6 |
| 294 | CTACCTGCACTATGAG | hsa-miR-20b | 51.2 |
| 295 | CTACCTGCACTATGAGC | hsa-miR-20b | 56 |
| 296 | CTACCTGCACTATGAGCA | hsa-miR-20b | 58.5 |
| 297 | TCAACATCAGTCTGATAAG | hsa-miR-21 | 53.4 |
| 298 | TCAACATCAGTCTGATAAGC | hsa-miR-21 | 57.3 |
| 299 | TCAACATCAGTCTGATAAGCT | hsa-miR-21 | 59 |
| 300 | TCAGCCGCTGTCAC | hsa-miR-210 | 55 |
| 301 | AGGCGAAGGATGACAAAG | hsa-miR-211 | 59.4 |
| 302 | GGCCGTGACTGG | hsa-miR-212 | 51.5 |
| 303 | GGCCGTGACTGGA | hsa-miR-212 | 55.1 |
| 304 | GGCCGTGACTGGAG | hsa-miR-212 | 57.6 |
| 305 | GGTACAATCAACGGT | hsa-miR-213 | 51.6 |
| 306 | GGTACAATCAACGGTC | hsa-miR-213 | 54.6 |
| 307 | GGTACAATCAACGGTCG | hsa-miR-213 | 58.9 |
| 308 | CTGCCTGTCTGTGC | hsa-miR-214 | 54.6 |
| 309 | GTCTGTCAATTCATAGG | hsa-miR-215 | 51 |
| 310 | GTCTGTCAATTCATAGGT | hsa-miR-215 | 53.9 |
| 311 | GTCTGTCAATTCATAGGTC | hsa-miR-215 | 56.3 |
| 312 | GTCTGTCAATTCATAGGTCA | hsa-miR-215 | 58.7 |
| 313 | GTCTGTCAATTCATAGGTCAT | hsa-miR-215 | 59.2 |
| 314 | CACAGTTGCCAGC | hsa-miR-216 | 50.9 |
| 315 | CACAGTTGCCAGCT | hsa-miR-216 | 53.5 |
| 316 | CACAGTTGCCAGCTG | hsa-miR-216 | 56.4 |
| 317 | CACAGTTGCCAGCTGA | hsa-miR-216 | 59.1 |
| 318 | ATCCAATCAGTTCCTG | hsa-miR-217 | 50.1 |
| 319 | ATCCAATCAGTTCCTGA | hsa-miR-217 | 53.1 |
| 320 | ATCCAATCAGTTCCTGAT | hsa-miR-217 | 54 |
| 321 | ATCCAATCAGTTCCTGATG | hsa-miR-217 | 56.6 |
| 322 | ACATGGTTAGATCAAGCAC | hsa-miR-218 | 56.8 |
| 323 | ACATGGTTAGATCAAGCACA | hsa-miR-218 | 59.1 |
| 324 | AGAATTGCGTTTGGA | hsa-miR-219 | 50.3 |
| 325 | AGAATTGCGTTTGGAC | hsa-miR-219 | 53.2 |
| 326 | AGAATTGCGTTTGGACA | hsa-miR-219 | 55.8 |
| 327 | AGAATTGCGTTTGGACAA | hsa-miR-219 | 57.3 |
| 328 | AGAATTGCGTTTGGACAAT | hsa-miR-219 | 57.9 |
| 329 | AGAATTGCGTTTGGACAATC | hsa-miR-219 | 60 |
| 330 | ACAGTTCTTCAACTGGC | hsa-miR-22 | 55.8 |
| 331 | ACAGTTCTTCAACTGGCA | hsa-miR-22 | 58.3 |
| 332 | AAAGTGTCAGATACGG | hsa-miR-220 | 50.5 |
| 333 | AAAGTGTCAGATACGGT | hsa-miR-220 | 53.4 |
| 334 | AAAGTGTCAGATACGGTG | hsa-miR-220 | 56 |
| 335 | AAAGTGTCAGATACGGTGT | hsa-miR-220 | 58.3 |
| 336 | GAAACCCAGCAGACAATG | hsa-miR-221 | 59.8 |
| 337 | GAGACCCAGTAGCCAG | hsa-miR-222 | 57.8 |
| 338 | GGGGTATTTGACAAACTGA | hsa-miR-223 | 59.2 |
| 339 | GGGGTATTTGACAAACTGAC | hsa-miR-223 | 61.4 |
| 340 | GGGGTATTTGACAAACTGACA | hsa-miR-223 | 63.4 |
| 341 | TAAACGGAACCACTAGTGA | hsa-miR-224 | 58.4 |
| 342 | TAAACGGAACCACTAGTGAC | hsa-miR-224 | 60.5 |
| 343 | TAAACGGAACCACTAGTGACT | hsa-miR-224 | 62 |
| 344 | GGAAATCCCTGGCAATG | hsa-miR-23a | 58.9 |
| 345 | GGTAATCCCTGGCAATG | hsa-miR-23b | 57.8 |
| 346 | CTGTTCCTGCTGAACTG | hsa-miR-24 | 58.1 |
| 347 | TCAGACCGAGACAAGTG | hsa-miR-25 | 57.6 |
| 348 | GCCTATCCTGGATTAC | hsa-miR-26a | 51.6 |
| 349 | GCCTATCCTGGATTACT | hsa-miR-26a | 53.9 |
| 350 | GCCTATCCTGGATTACTT | hsa-miR-26a | 55.6 |
| 351 | AACCTATCCTGAATTACT | hsa-miR-26b | 50.2 |
| 352 | AACCTATCCTGAATTACTT | hsa-miR-26b | 52 |
| 353 | AACCTATCCTGAATTACTTG | hsa-miR-26b | 54.5 |
| 354 | AACCTATCCTGAATTACTTGA | hsa-miR-26b | 56.7 |
| 355 | AACCTATCCTGAATTACTTGAA | hsa-miR-26b | 58 |
| 356 | GCGGAACTTAGCCAC | hsa-miR-27a | 56.3 |
| 357 | GCGGAACTTAGCCACT | hsa-miR-27a | 58.4 |
| 358 | GCAGAACTTAGCCACTG | hsa-miR-27b | 57.4 |
| 359 | GCAGAACTTAGCCACTGT | hsa-miR-27b | 59.8 |
| 360 | CTCAATAGACTGTGAGC | hsa-miR-28 | 53.8 |
| 361 | CTCAATAGACTGTGAGCT | hsa-miR-28 | 55.8 |
| 362 | CTCAATAGACTGTGAGCTC | hsa-miR-28 | 58.2 |
| 363 | ACAGGATTGAGGGGG | hsa-miR-296 | 54.7 |
| 364 | AAGCGGTTTACCATC | hsa-miR-299-3p | 50.4 |
| 365 | AAGCGGTTTACCATCC | hsa-miR-299-3p | 54.8 |
| 366 | AAGCGGTTTACCATCCC | hsa-miR-299-3p | 58.7 |
| 367 | ATGTATGTGGGACGG | hsa-miR-299-5p | 51.7 |
| 368 | ATGTATGTGGGACGGT | hsa-miR-299-5p | 54.8 |
| 369 | ATGTATGTGGGACGGTA | hsa-miR-299-5p | 55.4 |
| 370 | ATGTATGTGGGACGGTAA | hsa-miR-299-5p | 56.9 |
| 371 | ATGTATGTGGGACGGTAAA | hsa-miR-299-5p | 58.3 |
| 372 | AACCGATTTCAGATGGTG | hsa-miR-29a | 57.1 |
| 373 | AACACTGATTTCAAATGGTG | hsa-miR-29b | 57.2 |
| 374 | AACACTGATTTCAAATGGTGC | hsa-miR-29b | 60.6 |
| 375 | AACACTGATTTCAAATGGTGCT | hsa-miR-29b | 62.1 |
| 376 | ACCGATTTCAAATGGTGC | hsa-miR-29c | 58.3 |
| 377 | ACCGATTTCAAATGGTGCT | hsa-miR-29c | 60 |
| 378 | ACCGATTTCAAATGGTGCTA | hsa-miR-29c | 60.3 |
| 379 | GCTTTGACAATACTATTGC | hsa-miR-301 | 54.8 |
| 380 | GCTTTGACAATACTATTGCA | hsa-miR-301 | 57.1 |
| 381 | TCACCAAAACATGGAAGC | hsa-miR-302a | 58 |
| 382 | TCACCAAAACATGGAAGCA | hsa-miR-302a | 60.2 |
| 383 | TCACCAAAACATGGAAGCAC | hsa-miR-302a | 62.2 |
| 384 | AAAGCAAGTACATCCAC | hsa-miR-302a* | 52.6 |
| 385 | AAAGCAAGTACATCCACG | hsa-miR-302a* | 56.8 |
| 386 | AAAGCAAGTACATCCACGT | hsa-miR-302a* | 59 |
| 387 | CTACTAAAACATGGAAGCAC | hsa-miR-302b | 58.1 |
| 388 | CTACTAAAACATGGAAGCACT | hsa-miR-302b | 59.7 |
| 389 | AGAAAGCACTTCCATG | hsa-miR-302b* | 51.1 |
| 390 | AGAAAGCACTTCCATGT | hsa-miR-302b* | 54 |
| 391 | AGAAAGCACTTCCATGTT | hsa-miR-302b* | 55.6 |
| 392 | AGAAAGCACTTCCATGTTA | hsa-miR-302b* | 56.1 |
| 393 | AGAAAGCACTTCCATGTTAA | hsa-miR-302b* | 57.4 |
| 394 | AGAAAGCACTTCCATGTTAAA | hsa-miR-302b* | 58.7 |
| 395 | CCACTGAAACATGGA | hsa-miR-302c | 52 |
| 396 | CCACTGAAACATGGAA | hsa-miR-302c | 53.9 |
| 397 | CCACTGAAACATGGAAG | hsa-miR-302c | 56 |
| 398 | CAGCAGGTACCCCC | hsa-miR-302c* | 57 |
| 399 | ACACTCAAACATGGAAGCA | hsa-miR-302d | 59.4 |
| 400 | GCTGCAAACATCCGA | hsa-miR-30a-3p | 55.8 |
| 401 | GCTGCAAACATCCGAC | hsa-miR-30a-3p | 58.5 |
| 402 | CTTCCAGTCGAGGAT | hsa-miR-30a-5p | 53.4 |
| 403 | CTTCCAGTCGAGGATG | hsa-miR-30a-5p | 56.4 |
| 404 | CTTCCAGTCGAGGATGT | hsa-miR-30a-5p | 59 |
| 405 | AGCTGAGTGTAGGATG | hsa-miR-30b | 51.3 |
| 406 | AGCTGAGTGTAGGATGT | hsa-miR-30b | 54.3 |
| 407 | AGCTGAGTGTAGGATGTT | hsa-miR-30b | 55.9 |
| 408 | AGCTGAGTGTAGGATGTTT | hsa-miR-30b | 57.4 |
| 409 | AGCTGAGTGTAGGATGTTTA | hsa-miR-30b | 57.8 |
| 410 | GCTGAGAGTGTAGGATG | hsa-miR-30c | 55.7 |
| 411 | GCTGAGAGTGTAGGATGT | hsa-miR-30c | 58.3 |
| 412 | CTTCCAGTCGGGGA | hsa-miR-30d | 56.3 |
| 413 | CTTCCAGTCGGGGAT | hsa-miR-30d | 57.2 |
| 414 | GCTGTAAACATCCGAC | hsa-miR-30e-3p | 54.5 |
| 415 | GCTGTAAACATCCGACT | hsa-miR-30e-3p | 56.5 |
| 416 | GCTGTAAACATCCGACTG | hsa-miR-30e-3p | 59 |
| 417 | TCCAGTCAAGGATGTTTACA | hsa-miR-30e-5p | 59.4 |
| 418 | CAGCTATGCCAGCA | hsa-miR-31 | 52.2 |
| 419 | CAGCTATGCCAGCAT | hsa-miR-31 | 53.3 |
| 420 | CAGCTATGCCAGCATC | hsa-miR-31 | 56.1 |
| 421 | CAGCTATGCCAGCATCT | hsa-miR-31 | 58.1 |
| 422 | CAGCTATGCCAGCATCTT | hsa-miR-31 | 59.5 |
| 423 | GCAACTTAGTAATGTGCAA | hsa-miR-32 | 56.5 |
| 424 | GCAACTTAGTAATGTGCAAT | hsa-miR-32 | 57.1 |
| 425 | GCAACTTAGTAATGTGCAATA | hsa-miR-32 | 57.5 |
| 426 | TTCGCCCTCTCAAC | hsa-miR-320 | 52 |
| 427 | TTCGCCCTCTCAACC | hsa-miR-320 | 56.5 |
| 428 | TTCGCCCTCTCAACCC | hsa-miR-320 | 60.7 |
| 429 | AGAGGTCGACCGT | hsa-miR-323 | 50.7 |
| 430 | AGAGGTCGACCGTG | hsa-miR-323 | 54.1 |
| 431 | AGAGGTCGACCGTGT | hsa-miR-323 | 57.1 |
| 432 | AGAGGTCGACCGTGTA | hsa-miR-323 | 57.6 |
| 433 | AGAGGTCGACCGTGTAA | hsa-miR-323 | 59.1 |
| 434 | AGAGGTCGACCGTGTAAT | hsa-miR-323 | 59.7 |
| 435 | CCAGCAGCACCTGG | hsa-miR-324-3p | 57.8 |
| 436 | CCAGCAGCACCTGGG | hsa-miR-324-3p | 62.2 |
| 437 | CCAGCAGCACCTGGGG | hsa-miR-324-3p | 66.2 |
| 438 | ACACCAATGCCCTAGG | hsa-miR-324-5p | 56 |
| 439 | ACACTTACTGGACACC | hsa-miR-325 | 53.1 |
| 440 | ACACTTACTGGACACCT | hsa-miR-325 | 55.3 |
| 441 | ACACTTACTGGACACCTA | hsa-miR-325 | 55.8 |
| 442 | ACACTTACTGGACACCTAC | hsa-miR-325 | 58.2 |
| 443 | ACACTTACTGGACACCTACT | hsa-miR-325 | 59.9 |
| 444 | CTGGAGGAAGGGC | hsa-miR-326 | 52.3 |
| 445 | CTGGAGGAAGGGCC | hsa-miR-326 | 57.4 |
| 446 | ACGGAAGGGCAGAGAG | hsa-miR-328 | 59.2 |
| 447 | ACGGAAGGGCAGAGAGG | hsa-miR-328 | 63.2 |
| 448 | ACGGAAGGGCAGAGAGGG | hsa-miR-328 | 66.8 |
| 449 | AAAGAGGTTAACCAGG | hsa-miR-329 | 50.5 |
| 450 | AAAGAGGTTAACCAGGT | hsa-miR-329 | 53.5 |
| 451 | AAAGAGGTTAACCAGGTG | hsa-miR-329 | 56.2 |
| 452 | AAAGAGGTTAACCAGGTGT | hsa-miR-329 | 58.5 |
| 453 | CAATGCAACTACAATGCAC | hsa-miR-33 | 58.5 |
| 454 | TCTCTGCAGGCCG | hsa-miR-330 | 52.6 |
| 455 | TCTCTGCAGGCCGT | hsa-miR-330 | 55.8 |
| 456 | TCTCTGCAGGCCGTG | hsa-miR-330 | 58.8 |
| 457 | TTCTAGGATAGGCCCAGG | hsa-miR-331 | 59.3 |
| 458 | ACATTTTTCGTTATTGCTC | hsa-miR-335 | 54.1 |
| 459 | ACATTTTTCGTTATTGCTCT | hsa-miR-335 | 55.8 |
| 460 | ACATTTTTCGTTATTGCTCTT | hsa-miR-335 | 57.1 |
| 461 | ACATTTTTCGTTATTGCTCTTG | hsa-miR-335 | 59 |
| 462 | AAAGGCATCATATAGGAG | hsa-miR-337 | 51.2 |
| 463 | AAAGGCATCATATAGGAGC | hsa-miR-337 | 55.6 |
| 464 | AAAGGCATCATATAGGAGCT | hsa-miR-337 | 57.4 |
| 465 | AAAGGCATCATATAGGAGCTG | hsa-miR-337 | 59.6 |
| 466 | TCAACAAAATCACTGATGCTG | hsa-miR-338 | 60 |
| 467 | TCAACAAAATCACTGATGCTGG | hsa-miR-338 | 62.9 |
| 468 | TCAACAAAATCACTGATGCTGGA | hsa-miR-338 | 64.6 |
| 469 | TGAGCTCCTGGAGGAC | hsa-miR-339 | 58.5 |
| 470 | GGCTATAAAGTAACTGAGAC | hsa-miR-340 | 56.2 |
| 471 | GGCTATAAAGTAACTGAGACG | hsa-miR-340 | 59.8 |
| 472 | GACGGGTGCGATTTC | hsa-miR-342 | 57.6 |
| 473 | GACGGGTGCGATTTCT | hsa-miR-342 | 59.7 |
| 474 | GCCCTGGACTAGGAG | hsa-miR-345 | 56.4 |
| 475 | GCCCTGGACTAGGAGT | hsa-miR-345 | 59.3 |
| 476 | AGAGGCAGGCATGC | hsa-miR-346 | 53.9 |
| 477 | AGAGGCAGGCATGCG | hsa-miR-346 | 58.8 |
| 478 | AACAACCAGCTAAGACACT | hsa-miR-34a | 58.1 |
| 479 | AACAACCAGCTAAGACACTG | hsa-miR-34a | 60.3 |
| 480 | AACAACCAGCTAAGACACTGC | hsa-miR-34a | 63.7 |
| 481 | CAATCAGCTAATGACAC | hsa-miR-34b | 52.3 |
| 482 | CAATCAGCTAATGACACT | hsa-miR-34b | 54.4 |
| 483 | CAATCAGCTAATGACACTG | hsa-miR-34b | 56.8 |
| 484 | GCAATCAGCTAACTACAC | hsa-miR-34c | 55.4 |
| 485 | GCAATCAGCTAACTACACT | hsa-miR-34c | 57.2 |
| 486 | GTACCCCTGGAGATT | hsa-miR-361 | 52.2 |
| 487 | CTCACACCTAGGTTC | hsa-miR-362 | 51.3 |
| 488 | CTCACACCTAGGTTCC | hsa-miR-362 | 55.8 |
| 489 | CTCACACCTAGGTTCCA | hsa-miR-362 | 58.5 |
| 490 | CTCACACCTAGGTTCCAA | hsa-miR-362 | 59.9 |
| 491 | TTACAGATGGATACCGT | hsa-miR-363 | 52.4 |
| 492 | TTACAGATGGATACCGTG | hsa-miR-363 | 55.1 |
| 493 | TTACAGATGGATACCGTGC | hsa-miR-363 | 59.1 |
| 494 | ATAAGGATTTTTAGGGGC | hsa-miR-365 | 53.2 |
| 495 | ATAAGGATTTTTAGGGGCA | hsa-miR-365 | 55.8 |
| 496 | ATAAGGATTTTTAGGGGCAT | hsa-miR-365 | 56.5 |
| 497 | ATAAGGATTTTTAGGGGCATT | hsa-miR-365 | 57.8 |
| 498 | ATAAGGATTTTTAGGGGCATTA | hsa-miR-365 | 58.2 |
| 499 | TCACCATTGCTAAAGTGC | hsa-miR-367 | 56.9 |
| 500 | TCACCATTGCTAAAGTGCA | hsa-miR-367 | 59.2 |
| 501 | AAACGTGGAATTTCCTCTATG | hsa-miR-368 | 59.5 |
| 502 | AAAGATCAACCATGTATTA | hsa-miR-369-3p | 50.3 |
| 503 | AAAGATCAACCATGTATTAT | hsa-miR-369-3p | 51.2 |
| 504 | AAAGATCAACCATGTATTATT | hsa-miR-369-3p | 52.8 |
| 505 | GCGAATATAACACGGT | hsa-miR-369-5p | 52.9 |
| 506 | GCGAATATAACACGGTC | hsa-miR-369-5p | 55.5 |
| 507 | GCGAATATAACACGGTCG | hsa-miR-369-5p | 59.4 |
| 508 | CCAGGTTCCACCCC | hsa-miR-370 | 58.4 |
| 509 | ACACTCAAAAGATGGC | hsa-miR-371 | 51.8 |
| 510 | ACACTCAAAAGATGGCG | hsa-miR-371 | 56.2 |
| 511 | ACACTCAAAAGATGGCGG | hsa-miR-371 | 59.9 |
| 512 | ACGCTCAAATGTCGC | hsa-miR-372 | 54.7 |
| 513 | ACGCTCAAATGTCGCA | hsa-miR-372 | 57.4 |
| 514 | ACGCTCAAATGTCGCAG | hsa-miR-372 | 59.2 |
| 515 | ACACCCCAAAATCGAA | hsa-miR-373 | 54.6 |
| 516 | ACACCCCAAAATCGAAG | hsa-miR-373 | 56.7 |
| 517 | GGAAAGCGCCCCC | hsa-miR-373* | 58.2 |
| 518 | CACTTATCAGGTTGTATTATAA | hsa-miR-374 | 55 |
| 519 | TCACGCGAGCCG | hsa-miR-375 | 53.3 |
| 520 | TCACGCGAGCCGA | hsa-miR-375 | 56.5 |
| 521 | TCACGCGAGCCGAA | hsa-miR-375 | 58.2 |
| 522 | ACGTGGATTTTCCTCTATG | hsa-miR-376a | 56.9 |
| 523 | ACGTGGATTTTCCTCTATGA | hsa-miR-376a | 59.1 |
| 524 | ACGTGGATTTTCCTCTATGAT | hsa-miR-376a | 59.7 |
| 525 | AACATGGATTTTCCTCT | hsa-miR-376b | 51.1 |
| 526 | AACATGGATTTTCCTCTA | hsa-miR-376b | 51.8 |
| 527 | AACATGGATTTTCCTCTAT | hsa-miR-376b | 52.8 |
| 528 | AACATGGATTTTCCTCTATG | hsa-miR-376b | 55.2 |
| 529 | AACATGGATTTTCCTCTATGA | hsa-miR-376b | 57.5 |
| 530 | AACATGGATTTTCCTCTATGAT | hsa-miR-376b | 58 |
| 531 | ACAAAAGTTGCCTTTGTG | hsa-miR-377 | 56 |
| 532 | ACAAAAGTTGCCTTTGTGT | hsa-miR-377 | 58.3 |
| 533 | ACACAGGACCTGGAGTC | hsa-miR-378 | 59.8 |
| 534 | TACGTTCCATAGTCTACC | hsa-miR-379 | 54.2 |
| 535 | TACGTTCCATAGTCTACCA | hsa-miR-379 | 56.7 |
| 536 | AAGATGTGGACCATATTA | hsa-miR-380-3p | 50.6 |
| 537 | AAGATGTGGACCATATTAC | hsa-miR-380-3p | 53.3 |
| 538 | AAGATGTGGACCATATTACA | hsa-miR-380-3p | 55.8 |
| 539 | AAGATGTGGACCATATTACAT | hsa-miR-380-3p | 56.4 |
| 540 | AAGATGTGGACCATATTACATA | hsa-miR-380-3p | 56.9 |
| 541 | GCGCATGTTCTATGG | hsa-miR-380-5p | 52.7 |
| 542 | GCGCATGTTCTATGGT | hsa-miR-380-5p | 55.6 |
| 543 | GCGCATGTTCTATGGTC | hsa-miR-380-5p | 58.1 |
| 544 | ACAGAGAGCTTGCC | hsa-miR-381 | 50.3 |
| 545 | ACAGAGAGCTTGCCC | hsa-miR-381 | 55 |
| 546 | ACAGAGAGCTTGCCCT | hsa-miR-381 | 57.3 |
| 547 | ACAGAGAGCTTGCCCTT | hsa-miR-381 | 58.8 |
| 548 | CGAATCCACCACG | hsa-miR-382 | 51.3 |
| 549 | CGAATCCACCACGA | hsa-miR-382 | 54.5 |
| 550 | CGAATCCACCACGAA | hsa-miR-382 | 56.2 |
| 551 | CGAATCCACCACGAAC | hsa-miR-382 | 58.8 |
| 552 | AGCCACAATCACCTTCTG | hsa-miR-383 | 58.9 |
| 553 | AGCCACAATCACCTTCTGA | hsa-miR-383 | 61.1 |
| 554 | AGCCACAATCACCTTCTGAT | hsa-miR-383 | 61.6 |
| 555 | TATGAACAATTTCTAGGAA | hsa-miR-384 | 50.5 |
| 556 | TATGAACAATTTCTAGGAAT | hsa-miR-384 | 51.4 |
| 557 | AGGGGTTCACCGA | hsa-miR-409-3p | 51.3 |
| 558 | AGGGGTTCACCGAG | hsa-miR-409-3p | 54.1 |
| 559 | AGGGGTTCACCGAGC | hsa-miR-409-3p | 59.2 |
| 560 | TGCAAAGTTGCTCG | hsa-miR-409-5p | 50.3 |
| 561 | TGCAAAGTTGCTCGG | hsa-miR-409-5p | 54.7 |
| 562 | TGCAAAGTTGCTCGGG | hsa-miR-409-5p | 58.7 |
| 563 | AACAGGCCATCTGTG | hsa-miR-410 | 52.4 |
| 564 | AACAGGCCATCTGTGT | hsa-miR-410 | 55.3 |
| 565 | AACAGGCCATCTGTGTT | hsa-miR-410 | 57 |
| 566 | AACAGGCCATCTGTGTTA | hsa-miR-410 | 57.4 |
| 567 | AACAGGCCATCTGTGTTAT | hsa-miR-410 | 58.1 |
| 568 | AACAGGCCATCTGTGTTATA | hsa-miR-410 | 58.5 |
| 569 | AACAGGCCATCTGTGTTATAT | hsa-miR-410 | 59 |
| 570 | ACGGCTAGTGGACC | hsa-miR-412 | 53.4 |
| 571 | ACGGCTAGTGGACCA | hsa-miR-412 | 56.6 |
| 572 | ACGGCTAGTGGACCAG | hsa-miR-412 | 58.7 |
| 573 | GGCCTTCTGACCCTAAG | hsa-miR-422a | 59.7 |
| 574 | GGCCTTCTGACCCTAAGT | hsa-miR-422a | 62.1 |
| 575 | GGCCTTCTGACCCTAAGTC | hsa-miR-422a | 64.3 |
| 576 | GGCCTTCTGACTCCA | hsa-miR-422b | 56.7 |
| 577 | GGCCTTCTGACTCCAA | hsa-miR-422b | 58.4 |
| 578 | CTGAGGGGCCTC | hsa-miR-423 | 49.8 |
| 579 | CTGAGGGGCCTCA | hsa-miR-423 | 53.7 |
| 580 | CTGAGGGGCCTCAG | hsa-miR-423 | 56.4 |
| 581 | TTCAAAACATGAATTGCTGCT | hsa-miR-424 | 59.7 |
| 582 | TTCAAAACATGAATTGCTGCTG | hsa-miR-424 | 61.6 |
| 583 | GGCGGACACGAC | hsa-miR-425 | 52.9 |
| 584 | GGCGGACACGACA | hsa-miR-425 | 56.3 |
| 585 | GGCGGACACGACAT | hsa-miR-425 | 57.2 |
| 586 | GGCGGACACGACATT | hsa-miR-425 | 58.8 |
| 587 | ACGGTTTTACCAGAC | hsa-miR-429 | 50.4 |
| 588 | ACGGTTTTACCAGACA | hsa-miR-429 | 53.4 |
| 589 | ACGGTTTTACCAGACAG | hsa-miR-429 | 55.5 |
| 590 | ACGGTTTTACCAGACAGT | hsa-miR-429 | 58 |
| 591 | ACGGTTTTACCAGACAGTA | hsa-miR-429 | 58.4 |
| 592 | ACGGTTTTACCAGACAGTAT | hsa-miR-429 | 58.9 |
| 593 | TGCATGACGGCCT | hsa-miR-431 | 51.9 |
| 594 | TGCATGACGGCCTG | hsa-miR-431 | 55.2 |
| 595 | TGCATGACGGCCTGC | hsa-miR-431 | 60 |
| 596 | CCACCCAATGACC | hsa-miR-432 | 50.1 |
| 597 | CCACCCAATGACCT | hsa-miR-432 | 52.9 |
| 598 | CCACCCAATGACCTA | hsa-miR-432 | 53.7 |
| 599 | CCACCCAATGACCTAC | hsa-miR-432 | 56.6 |
| 600 | CCACCCAATGACCTACT | hsa-miR-432 | 58.7 |
| 601 | AGACATGGAGGAGCC | hsa-miR-432* | 54.1 |
| 602 | AGACATGGAGGAGCCA | hsa-miR-432* | 57.2 |
| 603 | AGACATGGAGGAGCCAT | hsa-miR-432* | 57.9 |
| 604 | ACACCGAGGAGCC | hsa-miR-433 | 52.6 |
| 605 | ACACCGAGGAGCCC | hsa-miR-433 | 57.5 |
| 606 | ATGGGACATCCTACAT | hsa-miR-448 | 50.1 |
| 607 | ATGGGACATCCTACATA | hsa-miR-448 | 51 |
| 608 | ATGGGACATCCTACATAT | hsa-miR-448 | 52 |
| 609 | ATGGGACATCCTACATATG | hsa-miR-448 | 54.7 |
| 610 | ATGGGACATCCTACATATGC | hsa-miR-448 | 58.8 |
| 611 | ACCAGCTAACAATACAC | hsa-miR-449 | 51.5 |
| 612 | ACCAGCTAACAATACACT | hsa-miR-449 | 53.6 |
| 613 | ACCAGCTAACAATACACTG | hsa-miR-449 | 56.1 |
| 614 | ACCAGCTAACAATACACTGC | hsa-miR-449 | 59.9 |
| 615 | TATTAGGAACACATCGC | hsa-miR-450 | 52 |
| 616 | TATTAGGAACACATCGCA | hsa-miR-450 | 54.6 |
| 617 | TATTAGGAACACATCGCAA | hsa-miR-450 | 56.1 |
| 618 | TATTAGGAACACATCGCAAA | hsa-miR-450 | 57.4 |
| 619 | TATTAGGAACACATCGCAAAA | hsa-miR-450 | 58.6 |
| 620 | TATTAGGAACACATCGCAAAAA | hsa-miR-450 | 59.7 |
| 621 | AAACTCAGTAATGGTAAC | hsa-miR-451 | 50.5 |
| 622 | AAACTCAGTAATGGTAACG | hsa-miR-451 | 54.6 |
| 623 | AAACTCAGTAATGGTAACGG | hsa-miR-451 | 58.1 |
| 624 | GTCTCAGTTTCCTCTG | hsa-miR-452 | 52.8 |
| 625 | GTCTCAGTTTCCTCTGC | hsa-miR-452 | 57.5 |
| 626 | CTTCTTTGCAGATGAG | hsa-miR-452* | 51.1 |
| 627 | CTTCTTTGCAGATGAGA | hsa-miR-452* | 53.9 |
| 628 | CTTCTTTGCAGATGAGAC | hsa-miR-452* | 56.4 |
| 629 | CTTCTTTGCAGATGAGACT | hsa-miR-452* | 58.2 |
| 630 | CGAACTCACCACG | hsa-miR-453 | 51.2 |
| 631 | CGAACTCACCACGG | hsa-miR-453 | 55.9 |
| 632 | CGAACTCACCACGGA | hsa-miR-453 | 58.6 |
| 633 | AGAGGAGAGCCGTG | hsa-miR-485-3p | 52.8 |
| 634 | AGAGGAGAGCCGTGT | hsa-miR-485-3p | 56 |
| 635 | AGAGGAGAGCCGTGTA | hsa-miR-485-3p | 56.5 |
| 636 | AGAGGAGAGCCGTGTAT | hsa-miR-485-3p | 57.3 |
| 637 | AGAGGAGAGCCGTGTATG | hsa-miR-485-3p | 59.8 |
| 638 | GAATTCATCACGGCC | hsa-miR-485-5p | 53.9 |
| 639 | GAATTCATCACGGCCA | hsa-miR-485-5p | 56.7 |
| 640 | GAATTCATCACGGCCAG | hsa-miR-485-5p | 58.7 |
| 641 | TTGAGAGTGCCATTATC | hsa-miR-488 | 51.7 |
| 642 | TTGAGAGTGCCATTATCT | hsa-miR-488 | 53.8 |
| 643 | TTGAGAGTGCCATTATCTG | hsa-miR-488 | 56.3 |
| 644 | TTGAGAGTGCCATTATCTGG | hsa-miR-488 | 59.8 |
| 645 | GCTGCCGTATATGTG | hsa-miR-489 | 51.6 |
| 646 | GCTGCCGTATATGTGA | hsa-miR-489 | 54.5 |
| 647 | GCTGCCGTATATGTGAT | hsa-miR-489 | 55.3 |
| 648 | GCTGCCGTATATGTGATG | hsa-miR-489 | 57.9 |
| 649 | CAGCATGGAGTCCT | hsa-miR-490 | 51.6 |
| 650 | CAGCATGGAGTCCTC | hsa-miR-490 | 54.8 |
| 651 | CAGCATGGAGTCCTCC | hsa-miR-490 | 59.2 |
| 652 | TCCTCATGGAAGGGT | hsa-miR-491 | 53.1 |
| 653 | TCCTCATGGAAGGGTT | hsa-miR-491 | 55 |
| 654 | TCCTCATGGAAGGGTTC | hsa-miR-491 | 57.8 |
| 655 | AAGAATCTTGTCCCGC | hsa-miR-492 | 54.8 |
| 656 | AAGAATCTTGTCCCGCA | hsa-miR-492 | 57.5 |
| 657 | AAGAATCTTGTCCCGCAG | hsa-miR-492 | 59.4 |
| 658 | AATGAAAGCCTACCATG | hsa-miR-493 | 52 |
| 659 | AATGAAAGCCTACCATGT | hsa-miR-493 | 54.7 |
| 660 | AATGAAAGCCTACCATGTA | hsa-miR-493 | 55.2 |
| 661 | AATGAAAGCCTACCATGTAC | hsa-miR-493 | 57.5 |
| 662 | AATGAAAGCCTACCATGTACA | hsa-miR-493 | 59.7 |
| 663 | AAGAGGTTTCCCGT | hsa-miR-494 | 50.1 |
| 664 | AAGAGGTTTCCCGTG | hsa-miR-494 | 53.4 |
| 665 | AAGAGGTTTCCCGTGT | hsa-miR-494 | 56.3 |
| 666 | AAGAGGTTTCCCGTGTA | hsa-miR-494 | 56.8 |
| 667 | AAGAGGTTTCCCGTGTAT | hsa-miR-494 | 57.5 |
| 668 | AAGAGGTTTCCCGTGTATG | hsa-miR-494 | 59.9 |
| 669 | AAAGAAGTGCACCATG | hsa-miR-495 | 51.9 |
| 670 | AAAGAAGTGCACCATGT | hsa-miR-495 | 54.6 |
| 671 | AAAGAAGTGCACCATGTT | hsa-miR-495 | 56.2 |
| 672 | AAAGAAGTGCACCATGTTT | hsa-miR-495 | 57.6 |
| 673 | AAAGAAGTGCACCATGTTTG | hsa-miR-495 | 59.7 |
| 674 | GAGATTGGCCATGTA | hsa-miR-496 | 50 |
| 675 | GAGATTGGCCATGTAA | hsa-miR-496 | 52.1 |
| 676 | GAGATTGGCCATGTAAT | hsa-miR-496 | 53.1 |
| 677 | ACAAACCACAGTGTG | hsa-miR-497 | 50.6 |
| 678 | ACAAACCACAGTGTGC | hsa-miR-497 | 55.5 |
| 679 | ACAAACCACAGTGTGCT | hsa-miR-497 | 57.5 |
| 680 | ACAAACCACAGTGTGCTG | hsa-miR-497 | 59.9 |
| 681 | GAAAAACGCCCCC | hsa-miR-498 | 52.5 |
| 682 | GAAAAACGCCCCCT | hsa-miR-498 | 55 |
| 683 | GAAAAACGCCCCCTG | hsa-miR-498 | 58 |
| 684 | TTAAACATCACTGCAAG | hsa-miR-499 | 50.4 |
| 685 | TTAAACATCACTGCAAGT | hsa-miR-499 | 53.1 |
| 686 | TTAAACATCACTGCAAGTC | hsa-miR-499 | 55.5 |
| 687 | TTAAACATCACTGCAAGTCT | hsa-miR-499 | 57.2 |
| 688 | TTAAACATCACTGCAAGTCTT | hsa-miR-499 | 58.4 |
| 689 | TTAAACATCACTGCAAGTCTTA | hsa-miR-499 | 58.7 |
| 690 | TTAAACATCACTGCAAGTCTTAA | hsa-miR-499 | 59.8 |
| 691 | CAGAATCCTTGCCC | hsa-miR-500 | 51.6 |
| 692 | CAGAATCCTTGCCCA | hsa-miR-500 | 54.8 |
| 693 | CAGAATCCTTGCCCAG | hsa-miR-500 | 57 |
| 694 | TCTCACCCAGGGAC | hsa-miR-501 | 53.2 |
| 695 | TCTCACCCAGGGACA | hsa-miR-501 | 56.5 |
| 696 | TCTCACCCAGGGACAA | hsa-miR-501 | 58.2 |
| 697 | TCTCACCCAGGGACAAA | hsa-miR-501 | 59.7 |
| 698 | TAGCACCCAGATAGC | hsa-miR-502 | 50.1 |
| 699 | TAGCACCCAGATAGCA | hsa-miR-502 | 53.3 |
| 700 | TAGCACCCAGATAGCAA | hsa-miR-502 | 55.1 |
| 701 | TAGCACCCAGATAGCAAG | hsa-miR-502 | 57.1 |
| 702 | CTGCAGAACTGTTCC | hsa-miR-503 | 53.2 |
| 703 | CTGCAGAACTGTTCCC | hsa-miR-503 | 57.4 |
| 704 | ATAGAGTGCAGACCAG | hsa-miR-504 | 51.3 |
| 705 | ATAGAGTGCAGACCAGG | hsa-miR-504 | 55.7 |
| 706 | ATAGAGTGCAGACCAGGG | hsa-miR-504 | 59.7 |
| 707 | GAGGAAACCAGCAA | hsa-miR-505 | 50.3 |
| 708 | GAGGAAACCAGCAAG | hsa-miR-505 | 52.9 |
| 709 | GAGGAAACCAGCAAGT | hsa-miR-505 | 55.9 |
| 710 | GAGGAAACCAGCAAGTG | hsa-miR-505 | 58.5 |
| 711 | TCTACTCAGAAGGGTG | hsa-miR-506 | 51.6 |
| 712 | TCTACTCAGAAGGGTGC | hsa-miR-506 | 56.5 |
| 713 | TTCACTCCAAAAGGTG | hsa-miR-507 | 52.1 |
| 714 | TTCACTCCAAAAGGTGC | hsa-miR-507 | 56.7 |
| 715 | TTCACTCCAAAAGGTGCA | hsa-miR-507 | 59.2 |
| 716 | TCTACTCCAAAAGGCT | hsa-miR-508 | 51.5 |
| 717 | TCTACTCCAAAAGGCTA | hsa-miR-508 | 52.3 |
| 718 | TCTACTCCAAAAGGCTAC | hsa-miR-508 | 55 |
| 719 | TCTACTCCAAAAGGCTACA | hsa-miR-508 | 57.5 |
| 720 | TCTACTCCAAAAGGCTACAA | hsa-miR-508 | 58.8 |
| 721 | TCTACTCCAAAAGGCTACAAT | hsa-miR-508 | 59.3 |
| 722 | TCTACCCACAGACGT | hsa-miR-509 | 53 |
| 723 | TCTACCCACAGACGTA | hsa-miR-509 | 53.7 |
| 724 | TCTACCCACAGACGTAC | hsa-miR-509 | 56.5 |
| 725 | TGTGATTGCCACTCT | hsa-miR-510 | 50.9 |
| 726 | TGTGATTGCCACTCTC | hsa-miR-510 | 53.9 |
| 727 | TGTGATTGCCACTCTCC | hsa-miR-510 | 58 |
| 728 | TGTGATTGCCACTCTCCT | hsa-miR-510 | 59.8 |
| 729 | TGACTGCAGAGCAAA | hsa-miR-511 | 51.8 |
| 730 | TGACTGCAGAGCAAAA | hsa-miR-511 | 53.7 |
| 731 | TGACTGCAGAGCAAAAG | hsa-miR-511 | 55.8 |
| 732 | TGACTGCAGAGCAAAAGA | hsa-miR-511 | 58.1 |
| 733 | GACCTCAGCTATGAC | hsa-miR-512-3p | 50.2 |
| 734 | GACCTCAGCTATGACA | hsa-miR-512-3p | 53.4 |
| 735 | GACCTCAGCTATGACAG | hsa-miR-512-3p | 55.7 |
| 736 | GAAAGTGCCCTCAA | hsa-miR-512-5p | 50.3 |
| 737 | GAAAGTGCCCTCAAG | hsa-miR-512-5p | 52.9 |
| 738 | GAAAGTGCCCTCAAGG | hsa-miR-512-5p | 57.3 |
| 739 | ATAAATGACACCTCCCT | hsa-miR-513 | 52.4 |
| 740 | ATAAATGACACCTCCCTG | hsa-miR-513 | 55.2 |
| 741 | ATAAATGACACCTCCCTGT | hsa-miR-513 | 57.6 |
| 742 | ATAAATGACACCTCCCTGTG | hsa-miR-513 | 59.9 |
| 743 | CTACTCACAGAAGTGT | hsa-miR-514 | 51 |
| 744 | CTACTCACAGAAGTGTC | hsa-miR-514 | 53.8 |
| 745 | CTACTCACAGAAGTGTCA | hsa-miR-514 | 56.4 |
| 746 | CTACTCACAGAAGTGTCAA | hsa-miR-514 | 57.8 |
| 747 | CTACTCACAGAAGTGTCAAT | hsa-miR-514 | 58.4 |
| 748 | ACGCTCCAAAAGAAG | hsa-miR-515-3p | 50.8 |
| 749 | ACGCTCCAAAAGAAGG | hsa-miR-515-3p | 55.1 |
| 750 | ACGCTCCAAAAGAAGGC | hsa-miR-515-3p | 59.4 |
| 751 | CAGAAAGTGCTTTCTT | hsa-miR-515-5p | 51 |
| 752 | CAGAAAGTGCTTTCTTT | hsa-miR-515-5p | 52.8 |
| 753 | CAGAAAGTGCTTTCTTTT | hsa-miR-515-5p | 54.4 |
| 754 | CAGAAAGTGCTTTCTTTTG | hsa-miR-515-5p | 56.8 |
| 755 | ACCCTCTGAAAGGAA | hsa-miR-516-3p | 50.6 |
| 756 | ACCCTCTGAAAGGAAG | hsa-miR-516-3p | 53.1 |
| 757 | ACCCTCTGAAAGGAAGC | hsa-miR-516-3p | 57.8 |
| 758 | AAAGTGCTTCTTACCTC | hsa-miR-516-5p | 52.1 |
| 759 | AAAGTGCTTCTTACCTCC | hsa-miR-516-5p | 56 |
| 760 | AAAGTGCTTCTTACCTCCA | hsa-miR-516-5p | 58.4 |
| 761 | AGACAGTGCTTCCAT | hsa-miR-517* | 50.1 |
| 762 | AGACAGTGCTTCCATC | hsa-miR-517* | 53.2 |
| 763 | AGACAGTGCTTCCATCT | hsa-miR-517* | 55.4 |
| 764 | AGACAGTGCTTCCATCTA | hsa-miR-517* | 55.9 |
| 765 | AGACAGTGCTTCCATCTAG | hsa-miR-517* | 57.8 |
| 766 | AACACTCTAAAGGGATG | hsa-miR-517a | 51.2 |
| 767 | AACACTCTAAAGGGATGC | hsa-miR-517a | 55.8 |
| 768 | AACACTCTAAAGGGATGCA | hsa-miR-517a | 58.2 |
| 769 | ACACTCTAAAAGGATGC | hsa-miR-517c | 51.9 |
| 770 | ACACTCTAAAAGGATGCA | hsa-miR-517c | 54.6 |
| 771 | ACACTCTAAAAGGATGCAC | hsa-miR-517c | 57 |
| 772 | TCCAGCAAAGGGAA | hsa-miR-518a | 50.3 |
| 773 | TCCAGCAAAGGGAAG | hsa-miR-518a | 52.9 |
| 774 | TCCAGCAAAGGGAAGC | hsa-miR-518a | 57.8 |
| 775 | AAAGGGCTTCCCTTT | hsa-miR-518a-2* | 52.1 |
| 776 | AAAGGGCTTCCCTTTG | hsa-miR-518a-2* | 55.1 |
| 777 | AAAGGGCTTCCCTTTGC | hsa-miR-518a-2* | 59.6 |
| 778 | ACCTCTAAAGGGGAG | hsa-miR-518b | 50 |
| 779 | ACCTCTAAAGGGGAGC | hsa-miR-518b | 55.4 |
| 780 | ACCTCTAAAGGGGAGCG | hsa-miR-518b | 59.9 |
| 781 | CACTCTAAAGAGAAGC | hsa-miR-518c | 50.1 |
| 782 | CACTCTAAAGAGAAGCG | hsa-miR-518c | 54.7 |
| 783 | CACTCTAAAGAGAAGCGC | hsa-miR-518c | 58.8 |
| 784 | CAGAAAGTGCTTCCC | hsa-miR-518c* | 53.5 |
| 785 | CAGAAAGTGCTTCCCT | hsa-miR-518c* | 55.8 |
| 786 | CAGAAAGTGCTTCCCTC | hsa-miR-518c* | 58.3 |
| 787 | GCTCCAAAGGGAAG | hsa-miR-518d | 51 |
| 788 | GCTCCAAAGGGAAGC | hsa-miR-518d | 56.5 |
| 789 | ACACTCTGAAGGGAA | hsa-miR-518e | 50.3 |
| 790 | ACACTCTGAAGGGAAG | hsa-miR-518e | 52.8 |
| 791 | ACACTCTGAAGGGAAGC | hsa-miR-518e | 57.5 |
| 792 | TCCTCTAAAGAGAAGCG | hsa-miR-518f | 54.1 |
| 793 | TCCTCTAAAGAGAAGCGC | hsa-miR-518f | 58.4 |
| 794 | AGAGAAAGTGCTTCCC | hsa-miR-518f* | 53.7 |
| 795 | AGAGAAAGTGCTTCCCT | hsa-miR-518f* | 55.9 |
| 796 | AGAGAAAGTGCTTCCCTC | hsa-miR-518f* | 58.4 |
| 797 | GTAACACTCTAAAAGGA | hsa-miR-519a | 50 |
| 798 | GTAACACTCTAAAAGGAT | hsa-miR-519a | 51.1 |
| 799 | GTAACACTCTAAAAGGATG | hsa-miR-519a | 53.7 |
| 800 | GTAACACTCTAAAAGGATGC | hsa-miR-519a | 57.7 |
| 801 | GTAACACTCTAAAAGGATGCA | hsa-miR-519a | 59.8 |
| 802 | AAACCTCTAAAAGGATGC | hsa-miR-519b | 53.9 |
| 803 | AAACCTCTAAAAGGATGCA | hsa-miR-519b | 56.4 |
| 804 | AAACCTCTAAAAGGATGCAC | hsa-miR-519b | 58.6 |
| 805 | ATCCTCTAAAAAGATGCA | hsa-miR-519c | 51.8 |
| 806 | ATCCTCTAAAAAGATGCAC | hsa-miR-519c | 54.4 |
| 807 | ATCCTCTAAAAAGATGCACT | hsa-miR-519c | 56.2 |
| 808 | ATCCTCTAAAAAGATGCACTT | hsa-miR-519c | 57.5 |
| 809 | ATCCTCTAAAAAGATGCACTTT | hsa-miR-519c | 58.7 |
| 810 | ACACTCTAAAGGGAGG | hsa-miR-519d | 51.9 |
| 811 | ACACTCTAAAGGGAGGC | hsa-miR-519d | 56.8 |
| 812 | ACACTCTAAAGGGAGGCA | hsa-miR-519d | 59.3 |
| 813 | ACACTCTAAAAGGAGGC | hsa-miR-519e | 54.4 |
| 814 | ACACTCTAAAAGGAGGCA | hsa-miR-519e | 57 |
| 815 | ACACTCTAAAAGGAGGCAC | hsa-miR-519e | 59.3 |
| 816 | GAAAGTGCTCCCTTT | hsa-miR-519e* | 51.8 |
| 817 | GAAAGTGCTCCCTTTT | hsa-miR-519e* | S3.7 |
| 818 | GAAAGTGCTCCCTTTTG | hsa-miR-519e* | 56.5 |
| 819 | ACAGTCCAAAGGGAA | hsa-miR-520a | 51.4 |
| 820 | ACAGTCCAAAGGGAAG | hsa-miR-520a | 53.8 |
| 821 | ACAGTCCAAAGGGAAGC | hsa-miR-520a | 58.5 |
| 822 | AGAAAGTACTTCCCTCT | hsa-miR-520a* | 51.7 |
| 823 | AGAAAGTACTTCCCTCTG | hsa-miR-520a* | 54.6 |
| 824 | AGAAAGTACTTCCCTCTGG | hsa-miR-520a* | 58.4 |
| 825 | CCCTCTAAAAGGAAGC | hsa-miR-520b | 53.8 |
| 826 | CCCTCTAAAAGGAAGCA | hsa-miR-520b | 56.6 |
| 827 | CCCTCTAAAAGGAAGCAC | hsa-miR-520b | 59 |
| 828 | AACCCTCTAAAAGGAAG | hsa-miR-520c | 51.7 |
| 829 | AACCCTCTAAAAGGAAGC | hsa-miR-520c | 56.3 |
| 830 | AACCCTCTAAAAGGAAGCA | hsa-miR-520c | 58.7 |
| 831 | AACCCACCAAAGAGA | hsa-miR-520d | 51.4 |
| 832 | AACCCACCAAAGAGAA | hsa-miR-520d | 53.4 |
| 833 | AACCCACCAAAGAGAAG | hsa-miR-520d | 55.6 |
| 834 | AACCCACCAAAGAGAAGC | hsa-miR-520d | 59.9 |
| 835 | CAGAAAGGGCTTCC | hsa-miR-520d* | 51.8 |
| 836 | CAGAAAGGGCTTCCC | hsa-miR-520d* | 56.5 |
| 837 | CAGAAAGGGCTTCCCT | hsa-miR-520d* | 58.6 |
| 838 | CCCTCAAAAAGGAAG | hsa-miR-520e | 50.6 |
| 839 | CCCTCAAAAAGGAAGC | hsa-miR-520e | 55.6 |
| 840 | CCCTCAAAAAGGAAGCA | hsa-miR-520e | 58.3 |
| 841 | ACACTCTAAAGGGAAGC | hsa-miR-520g | 54.4 |
| 842 | ACACTCTAAAGGGAAGCA | hsa-miR-520g | 57 |
| 843 | ACACTCTAAAGGGAAGCAC | hsa-miR-520g | 59.3 |
| 844 | ACTCTAAAGGGAAGCA | hsa-miR-520h | 51.5 |
| 845 | ACTCTAAAGGGAAGCAC | hsa-miR-520h | 54.4 |
| 846 | ACTCTAAAGGGAAGCACT | hsa-miR-520h | 56.4 |
| 847 | ACTCTAAAGGGAAGCACTT | hsa-miR-520h | 57.9 |
| 848 | ACTCTAAAGGGAAGCACTTT | hsa-miR-520h | 59.1 |
| 849 | ACACTCTAAAGGGAAGT | hsa-miR-521 | 52.5 |
| 850 | ACACTCTAAAGGGAAGTG | hsa-miR-521 | 55.2 |
| 851 | ACACTCTAAAGGGAAGTGC | hsa-miR-521 | 59.3 |
| 852 | AACACTCTAAAGGGAAC | hsa-miR-522 | 52.1 |
| 853 | AACACTCTAAAGGGAACC | hsa-miR-522 | 56.1 |
| 854 | AACACTCTAAAGGGAACCA | hsa-miR-522 | 58.5 |
| 855 | AACACTCTAAAGGGAACCAT | hsa-miR-522 | 59.1 |
| 856 | CCCTCTATAGGGAAGC | hsa-miR-523 | 54.3 |
| 857 | CCCTCTATAGGGAAGCG | hsa-miR-523 | 58.9 |
| 858 | ACTCCAAAGGGAAGC | hsa-miR-524 | 52.8 |
| 859 | ACTCCAAAGGGAAGCG | hsa-miR-524 | 57.6 |
| 860 | GAGAAAGTGCTTCCC | hsa-miR-524* | 52.8 |
| 861 | GAGAAAGTGCTTCCCT | hsa-miR-524* | 55.2 |
| 862 | GAGAAAGTGCTTCCCTT | hsa-miR-524* | 56.8 |
| 863 | GAGAAAGTGCTTCCCTTT | hsa-miR-524* | 58.3 |
| 864 | AGAAAGTGCATCCCT | hsa-miR-525 | 50.4 |
| 865 | AGAAAGTGCATCCCTC | hsa-miR-525 | 53.5 |
| 866 | AGAAAGTGCATCCCTCT | hsa-miR-525 | 55.7 |
| 867 | AGAAAGTGCATCCCTCTG | hsa-miR-525 | 58.3 |
| 868 | GCTCTAAAGGGAAGC | hsa-miR-525* | 51.9 |
| 869 | GCTCTAAAGGGAAGCG | hsa-miR-525* | 56.7 |
| 870 | AGAAAGTGCTTCCCT | hsa-miR-526a | 50.6 |
| 871 | AGAAAGTGCTTCCCTC | hsa-miR-526a | 53.7 |
| 872 | AGAAAGTGCTTCCCTCT | hsa-miR-526a | 55.9 |
| 873 | AGAAAGTGCTTCCCTCTA | hsa-miR-526a | 56.4 |
| 874 | AGAAAGTGCTTCCCTCTAG | hsa-miR-526a | 58.3 |
| 875 | AACAGAAAGTGCTTCC | hsa-miR-526b | 52 |
| 876 | AACAGAAAGTGCTTCCC | hsa-miR-526b | 56.1 |
| 877 | AACAGAAAGTGCTTCCCT | hsa-miR-526b | 58 |
| 878 | GCCTCTAAAAGGAAGC | hsa-miR-526b* | 53.8 |
| 879 | GCCTCTAAAAGGAAGCA | hsa-miR-526b* | 56.6 |
| 880 | GCCTCTAAAAGGAAGCAC | hsa-miR-526b* | 59 |
| 881 | AACAGAAAGCGCTTC | hsa-miR-526c | 51.5 |
| 882 | AACAGAAAGCGCTTCC | hsa-miR-526c | 55.6 |
| 883 | AACAGAAAGCGCTTCCC | hsa-miR-526c | 59.4 |
| 884 | AGAAAGGGCTTCCC | hsa-miR-527 | 51 |
| 885 | AGAAAGGGCTTCCCT | hsa-miR-527 | 53.6 |
| 886 | AGAAAGGGCTTCCCTT | hsa-miR-527 | 55.5 |
| 887 | AGAAAGGGCTTCCCTTT | hsa-miR-527 | 57.1 |
| 888 | AGAAAGGGCTTCCCTTTG | hsa-miR-527 | 59.7 |
| 889 | CAACAAAATCACTAGTCTTCC | hsa-miR-7 | 59.5 |
| 890 | TCATACAGCTAGATAACCA | hsa-miR-9 | 53.4 |
| 891 | TCATACAGCTAGATAACCAA | hsa-miR-9 | 54.9 |
| 892 | TCATACAGCTAGATAACCAAA | hsa-miR-9 | 56.3 |
| 893 | TCATACAGCTAGATAACCAAAG | hsa-miR-9 | 57.9 |
| 894 | TCATACAGCTAGATAACCAAAGA | hsa-miR-9 | 59.8 |
| 895 | ACTTTCGGTTATCTAGC | hsa-miR-9* | 51.5 |
| 896 | ACTTTCGGTTATCTAGCT | hsa-miR-9* | 53.6 |
| 897 | ACTTTCGGTTATCTAGCTT | hsa-miR-9* | 55.1 |
| 898 | ACTTTCGGTTATCTAGCTTT | hsa-miR-9* | 56.5 |
| 899 | ACTTTCGGTTATCTAGCTTTA | hsa-miR-9* | 56.9 |
| 900 | CAGGCCGGGACAAG | hsa-miR-92 | 58.6 |
| 901 | CAGGCCGGGACAAGT | hsa-miR-92 | 61.3 |
| 902 | CAGGCCGGGACAAGTG | hsa-miR-92 | 63.8 |
| 903 | CTACCTGCACGAACAG | hsa-miR-93 | 56.7 |
| 904 | TGCTCAATAAATACCCG | hsa-miR-95 | 52.3 |
| 905 | TGCTCAATAAATACCCGT | hsa-miR-95 | 54.9 |
| 906 | TGCTCAATAAATACCCGTT | hsa-miR-95 | 56.4 |
| 907 | TGCTCAATAAATACCCGTTG | hsa-miR-95 | 58.6 |
| 908 | GCAAAAATGTGCTAGTGC | hsa-miR-96 | 57.7 |
| 909 | GCAAAAATGTGCTAGTGCC | hsa-miR-96 | 61.1 |
| 910 | GCAAAAATGTGCTAGTGCCA | hsa-miR-96 | 63.1 |
| 911 | AACAATACAACTTACTACCTC | hsa-miR-98 | 55.4 |
| 912 | CACAAGATCGGATCTACG | hsa-miR-99a | 58.4 |
| 913 | CGCAAGGTCGGTTC | hsa-miR-99b | 56.7 |
| 914 | CGCAAGGTCGGTTGT | hsa-miR-99b | 58.8 |
| 915 | CGCAAGGTCGGTTCTA | hsa-miR-99b | 59.2 |
| 916 | AACTATACAACCTACTACCTC | hsa-let-7a | 56 |
| 917 | AACTATACAACCTACTACCTCA | hsa-let-7a | 58.2 |
| 918 | AACCACACAACCTACT | hsa-let-7b | 52.1 |
| 919 | AACCACACAACCTACTACC | hsa-let-7b | 59.1 |
| 920 | AACCATACAACCTACTACCTC | hsa-let-7c | 59.5 |
| 921 | ACTATGCAACCTACTACC | hsa-let-7d | 53.8 |
| 922 | ACTATGCAACCTACTACCTC | hsa-let-7d | 58.1 |
| 923 | ACTATGCAACCTACTACCTCT | hsa-let-7d | 59.7 |
| 924 | ACTATACAACCTCCTACC | hsa-let-7e | 53.2 |
| 925 | ACTATACAACCTCCTACCT | hsa-let-7e | 55.2 |
| 926 | CACAAACCATTATGTGC | hsa-miR-15a | 53.9 |
| 927 | CACAAACCATTATGTGCT | hsa-miR-15a | 55.9 |
| 928 | CGCCAATATTTACGTG | hsa-miR-16 | 52.5 |
| 929 | ACAAGTGCCTTCACT | hsa-miR-17-3p | 51.2 |
| 930 | ACAAGTGCCTTCACTG | hsa-miR-17-3p | 54.2 |
| 931 | ACTACCTGCACTGTAA | hsa-miR-17-5p | 50.8 |
| 932 | ACTACCTGCACTGTAAG | hsa-miR-17-5p | 53.1 |
| 933 | TCAGTTTTGCATAGATTT | hsa-miR-19a | 50.4 |
| 934 | TCAGTTTTGCATAGATTTG | hsa-miR-19a | 53 |
| 935 | TCAGTTTTGCATGGAT | hsa-miR-19b | 50.6 |
| 936 | TCAGTTTTGCATGGATT | hsa-miR-19b | 52.5 |
| 937 | TCAGTTTTGCATGGATTT | hsa-miR-19b | 54.1 |
| 938 | TCAACATCAGTCTGATAA | hsa-miR-21 | 51.3 |
| 939 | TCAACATCAGTCTGATAAGCTA | hsa-miR-21 | 59.3 |
| 940 | ACAGTTCTTCAACTGG | hsa-miR-22 | 51.1 |
| 941 | GGAAATCCCTGGCA | hsa-miR-23a | 53.2 |
| 942 | GGAAATCCCTGGCAA | hsa-miR-23a | 55.2 |
| 943 | GGAAATCCCTGGCAAT | hsa-miR-23a | 56.1 |
| 944 | ACTGATATCAGCTCAGT | hsa-miR-189 | 51 |
| 945 | ACTGATATCAGCTCAGTA | hsa-miR-189 | 51.8 |
| 946 | ACTGATATCAGCTCAGTAG | hsa-miR-189 | 53.8 |
| 947 | CTGTTCCTGCTGAA | hsa-miR-24 | 50 |
| 948 | CTGTTCCTGCTGAAC | hsa-miR-24 | 53.2 |
| 949 | CTGTTCCTGCTGAACT | hsa-miR-24 | 55.4 |
| 950 | TCAGACCGAGACAAG | hsa-miR-25 | 52 |
| 951 | TCAGACCGAGACAAGT | hsa-miR-25 | 54.9 |
| 952 | GCCTATCCTGGATTACTTG | hsa-miR-26a | 58.2 |
| 953 | GCGGAACTTAGCCA | hsa-miR-27a | 53.3 |
| 954 | AACCGATTTCAGATGG | hsa-miR-29a | 51.7 |
| 955 | AACCGATTTCAGATGGT | hsa-miR-29a | 54.5 |
| 956 | GCTGCAAACATCCG | hsa-miR-30a-3p | 52.9 |
| 957 | CTTCCAGTCGAGGA | hsa-miR-30a-5p | 52.3 |
| 958 | GCAACTTAGTAATGTGC | hsa-miR-32 | 52.4 |
| 959 | GCAACTTAGTAATGTGCA | hsa-miR-32 | 55 |
| 960 | CAATGCAACTACAATGC | hsa-miR-33 | 53.9 |
| 961 | CAATGCAACTACAATGCA | hsa-miR-33 | 56.3 |
| 962 | CTACCTGCACGAAC | hsa-miR-93 | 51.5 |
| 963 | CTACCTGCACGAACA | hsa-miR-93 | 54.6 |
| 964 | GCAAAAATGTGCTAGT | hsa-miR-96 | 50.7 |
| 965 | GCAAAAATGTGCTAGTG | hsa-miR-96 | 53.5 |
| 966 | AACAATACAACTTACTACC | hsa-miR-98 | 51.3 |
| 967 | AACAATACAACTTACTACCT | hsa-miR-98 | 53.2 |
| 968 | AACAATACAACTTACTACCTCA | hsa-miR-98 | 57.6 |
| 969 | CACAAGATCGGATCT | hsa-miR-99a | 50.7 |
| 970 | CACAAGATCGGATCTA | hsa-miR-99a | 51.5 |
| 971 | CACAAGATCGGATCTAC | hsa-miR-99a | 54.3 |
| 972 | CACAAGTTCGGATCT | hsa-miR-100 | 51.7 |
| 973 | CACAAGTTCGGATCTA | hsa-miR-100 | 52.4 |
| 974 | CTTCAGTTATCACAGTAC | hsa-miR-101 | 52.3 |
| 975 | CTTCAGTTATCACAGTACT | hsa-miR-101 | 54.3 |
| 976 | CTTCAGTTATCACAGTACTG | hsa-miR-101 | 56.7 |
| 977 | CTTCAGTTATCACAGTACTGT | hsa-miR-101 | 58.8 |
| 978 | AACACTGATTTCAAATGG | hsa-miR-29b | 52.4 |
| 979 | AACACTGATTTCAAATGGT | hsa-miR-29b | 54.9 |
| 980 | TCATAGCCCTGTACAA | hsa-miR-103 | 50.9 |
| 981 | TCATAGCCCTGTACAATGC | hsa-miR-103 | 58.8 |
| 982 | GCTACCTGCACTGT | hsa-miR-106a | 51.5 |
| 983 | GCTACCTGCACTGTA | hsa-miR-106a | 52.3 |
| 984 | GCTACCTGCACTGTAA | hsa-miR-106a | 54.2 |
| 985 | TGATAGCCCTGTACAA | hsa-miR-107 | 50.9 |
| 986 | TGATAGCCCTGTACAAT | hsa-miR-107 | 51.9 |
| 987 | TGATAGCCCTGTACAATG | hsa-miR-107 | 54.7 |
| 988 | GAACAGGTAGTCTGAA | hsa-miR-199a | 51.2 |
| 989 | GAACAGGTAGTCTGAAC | hsa-miR-199a | 54.2 |
| 990 | GAACAGGTAGTCTGAACA | hsa-miR-199a | 56.8 |
| 991 | AACCAATGTGCAGAC | hsa-miR-199a* | 50.6 |
| 992 | AACCAATGTGCAGACT | hsa-miR-199a* | 52.9 |
| 993 | AACCAATGTGCAGACTA | hsa-miR-199a* | 53.6 |
| 994 | ACAAGCTTTTTGCTCGT | hsa-miR-208 | 55.9 |
| 995 | ACAAAGTTCTGTAGTGC | hsa-miR-148a | 52.5 |
| 996 | ACAAAGTTCTGTAGTGCA | hsa-miR-148a | 55.1 |
| 997 | GCTGAGAGTGTAGGA | hsa-miR-30c | 51.6 |
| 998 | GCTGAGAGTGTAGGAT | hsa-miR-30c | 52.7 |
| 999 | GCTGAGAGTGTAGGATGTT | hsa-miR-30c | 59.6 |
| 1000 | CTTCCAGTCGGGGATG | hsa-miR-30d | 60 |
| 1001 | AGACACGTGCACTG | hsa-miR-139 | 51.4 |
| 1002 | AGACACGTGCACTGT | hsa-miR-139 | 54.5 |
| 1003 | AGACACGTGCACTGTA | hsa-miR-139 | 55.1 |
| 1004 | GCAGAAGCATTTCCA | hsa-miR-147 | 52.2 |
| 1005 | CAACAAAATCACTAGTC | hsa-miR-7 | 50.4 |
| 1006 | CAACAAAATCACTAGTCT | hsa-miR-7 | 52.5 |
| 1007 | CAACAAAATCACTAGTCTT | hsa-miR-7 | 54 |
| 1008 | CAACAAAATCACTAGTCTTC | hsa-miR-7 | 56.3 |
| 1009 | CACAAATTCGGATCTA | hsa-miR-10a | 50.2 |
| 1010 | CACAAATTCGGATCTAC | hsa-miR-10a | 53 |
| 1011 | CACAAATTCGGATCTACA | hsa-miR-10a | 55.6 |
| 1012 | ACAAATTCGGTTCTACA | hsa-miR-10b | 52 |
| 1013 | ACAAATTCGGTTCTACAG | hsa-miR-10b | 54.1 |
| 1014 | AACAACCAGCTAAGAC | hsa-miR-34a | 50.9 |
| 1015 | AACAACCAGCTAAGACA | hsa-miR-34a | 53.8 |
| 1016 | AACAACCAGCTAAGACAC | hsa-miR-34a | 56.3 |
| 1017 | ACTCACCGACAGGT | hsa-miR-181c | 52.1 |
| 1018 | ACTCACCGACAGGTT | hsa-miR-181c | 54.2 |
| 1019 | ACTCACCGACAGGTTG | hsa-miR-181c | 57.1 |
| 1020 | TGTGAGTTCTACCATTGCC | hsa-miR-182 | 59.7 |
| 1021 | GAACAGATAGTCTAAACA | hsa-miR-199b | 50.8 |
| 1022 | GAACAGATAGTCTAAACAC | hsa-miR-199b | 53.4 |
| 1023 | GAACAGATAGTCTAAACACT | hsa-miR-199b | 55.3 |
| 1024 | AGGCATAGGATGACAA | hsa-miR-204 | 51 |
| 1025 | AGGCATAGGATGACAAA | hsa-miR-204 | 53 |
| 1026 | AGGCATAGGATGACAAAG | hsa-miR-204 | 55.1 |
| 1027 | TCAGCCGCTGTCACA | hsa-miR-210 | 57.9 |
| 1028 | AGGCGAAGGATGAC | hsa-miR-211 | 50.9 |
| 1029 | AGGCGAAGGATGACA | hsa-miR-211 | 54.1 |
| 1030 | AGGCGAAGGATGACAA | hsa-miR-211 | 55.9 |
| 1031 | AGGCGAAGGATGACAAA | hsa-miR-211 | 57.5 |
| 1032 | CTGCCTGTCTGTGCC | hsa-miR-214 | 59 |
| 1033 | ACATGGTTAGATCAAGC | hsa-miR-218 | 51.7 |
| 1034 | ACATGGTTAGATCAAGCA | hsa-miR-218 | 54.4 |
| 1035 | GAAACCCAGCAGAC | hsa-miR-221 | 51.5 |
| 1036 | GAAACCCAGCAGACA | hsa-miR-221 | 54.8 |
| 1037 | GAAACCCAGCAGACAA | hsa-miR-221 | 56.5 |
| 1038 | GAAACCCAGCAGACAAT | hsa-miR-221 | 57.3 |
| 1039 | GAGACCCAGTAGCC | hsa-miR-222 | 52.1 |
| 1040 | GAGACCCAGTAGCCA | hsa-miR-222 | 55.5 |
| 1041 | GGGGTATTTGACAAAC | hsa-miR-223 | 52.1 |
| 1042 | GGGGTATTTGACAAACT | hsa-miR-223 | 54.3 |
| 1043 | GGGGTATTTGACAAACTG | hsa-miR-223 | 56.9 |
| 1044 | TAAACGGAACCACTAG | hsa-miR-224 | 50.6 |
| 1045 | TAAACGGAACCACTAGT | hsa-miR-224 | 53.5 |
| 1046 | TAAACGGAACCACTAGTG | hsa-miR-224 | 56.1 |
| 1047 | ACTGTACAAACTACTACC | hsa-let-7g | 51.6 |
| 1048 | ACAGCACAAACTACTAC | hsa-let-7i | 51.5 |
| 1049 | GGTAATCCCTGGCA | hsa-miR-23b | 51.9 |
| 1050 | GGTAATCCCTGGCAA | hsa-miR-23b | 54 |
| 1051 | GGTAATCCCTGGCAAT | hsa-miR-23b | 55 |
| 1052 | GCAGAACTTAGCCAC | hsa-miR-27b | 52.4 |
| 1053 | GCAGAACTTAGCCACT | hsa-miR-27b | 54.7 |
| 1054 | AGCTGAGTGTAGGATGTTTAC | hsa-miR-30b | 59.9 |
| 1055 | TCACAAGTTAGGGTCT | hsa-miR-125b | 51.3 |
| 1056 | TCACAAGTTAGGGTCTCAG | hsa-miR-125b | 58.7 |
| 1057 | ATGCCCTTTTAACATTG | hsa-miR-130a | 50.7 |
| 1058 | ATGCCCTTTTAACATTGCAC | hsa-miR-130a | 59.6 |
| 1059 | CGACCATGGCTGTA | hsa-miR-132 | 52.8 |
| 1060 | ACAGCTGGTTGAAGG | hsa-miR-133a | 52.6 |
| 1061 | TCACATAGGAATAAAAAGC | hsa-miR-135a | 52.2 |
| 1062 | TCACATAGGAATAAAAAGCC | hsa-miR-135a | 55.8 |
| 1063 | CTACCATAGGGTAAAAC | hsa-miR-140 | 51 |
| 1064 | CTACCATAGGGTAAAACC | hsa-miR-140 | 55 |
| 1065 | TCCATAAAGTAGGAAACA | hsa-miR-142-3p | 51.7 |
| 1066 | TCCATAAAGTAGGAAACAC | hsa-miR-142-3p | 54.3 |
| 1067 | TCCATAAAGTAGGAAACACT | hsa-miR-142-3p | 56.1 |
| 1068 | TCCATAAAGTAGGAAACACTA | hsa-miR-142-3p | 56.6 |
| 1069 | GTAGTGCTTTCTACTTT | hsa-miR-142-5p | 50.1 |
| 1070 | GTAGTGCTTTCTACTTTA | hsa-miR-142-5p | 50.9 |
| 1071 | TGAGCTACAGTGCTTC | hsa-miR-143 | 52.9 |
| 1072 | TGAGCTACAGTGCTTCA | hsa-miR-143 | 55.7 |
| 1073 | TGAGCTACAGTGCTTCAT | hsa-miR-143 | 56.4 |
| 1074 | CTAGTACATCATCTATACTG | hsa-miR-144 | 51.5 |
| 1075 | CTAGTACATCATCTATACTGT | hsa-miR-144 | 53.9 |
| 1076 | AAGGGATTCCTGGGA | hsa-miR-145 | 53.4 |
| 1077 | AAGGGATTCCTGGGAA | hsa-miR-145 | 55.3 |
| 1078 | AAGGGATTCCTGGGAAA | hsa-miR-145 | 57.1 |
| 1079 | CCCAAGTTCTGTCA | hsa-miR-152 | 50 |
| 1080 | CCCAAGTTCTGTCAT | hsa-miR-152 | 51.2 |
| 1081 | CCCAAGTTCTGTCATG | hsa-miR-152 | 54.3 |
| 1082 | AGCTGCTTTTGGGATTC | hsa-miR-191 | 56.1 |
| 1083 | AGCTGCTTTTGGGATTCC | hsa-miR-191 | 59.9 |
| 1084 | TCATACAGCTAGATAACC | hsa-miR-9 | 50.7 |
| 1085 | CACAGGTTAAAGGGT | hsa-miR-125a | 51.4 |
| 1086 | CACAGGTTAAAGGGTC | hsa-miR-125a | 54.3 |
| 1087 | CACAGGTTAAAGGGTCT | hsa-miR-125a | 56.4 |
| 1088 | GCATTATTACTCACGG | hsa-miR-126 | 50.4 |
| 1089 | GCATTATTACTCACGGT | hsa-miR-126 | 53.3 |
| 1090 | GCATTATTACTCACGGTA | hsa-miR-126 | 53.9 |
| 1091 | CGCGTACCAAAAGT | hsa-miR-126* | 52 |
| 1092 | CGCGTACCAAAAGTA | hsa-miR-126* | 52.8 |
| 1093 | CGCGTACCAAAAGTAA | hsa-miR-126* | 54.5 |
| 1094 | CGCGTACCAAAAGTAAT | hsa-miR-126* | 55.3 |
| 1095 | CGCGTACCAAAAGTAATA | hsa-miR-126* | 55.8 |
| 1096 | CGCGTACCAAAAGTAATAA | hsa-miR-126* | 57.1 |
| 1097 | CGCGTACCAAAAGTAATAAT | hsa-miR-126* | 57.7 |
| 1098 | AGCCAAGCTCAGAC | hsa-miR-127 | 50.3 |
| 1099 | AGCCAAGCTCAGACG | hsa-miR-127 | 55.4 |
| 1100 | CCCTCTGGTCAACCAG | hsa-miR-134 | 59.6 |
| 1101 | TCCATCATCAAAACAAA | hsa-miR-136 | 50.3 |
| 1102 | TCCATCATCAAAACAAAT | hsa-miR-136 | 51.2 |
| 1103 | TCCATCATCAAAACAAATG | hsa-miR-136 | 53.8 |
| 1104 | TCCATCATCAAAACAAATGG | hsa-miR-136 | 57.3 |
| 1105 | ACCCTTATCAGTTCTCC | hsa-miR-184 | 53.6 |
| 1106 | GAACTGCCTTTCTCTC | hsa-miR-185 | 53.7 |
| 1107 | GAACTGCCTTTCTCTCC | hsa-miR-185 | 57.8 |
| 1108 | AAGCCCAAAAGGAGA | hsa-miR-186 | 51.7 |
| 1109 | AAGCCCAAAAGGAGAA | hsa-miR-186 | 53.7 |
| 1110 | AAGCCCAAAAGGAGAAT | hsa-miR-186 | 54.6 |
| 1111 | AAGCCCAAAAGGAGAATT | hsa-miR-186 | 56.2 |
| 1112 | ACCCTCCACCATGCAA | hsa-miR-188 | 58.9 |
| 1113 | TCCACATGGAGTTGCTG | hsa-miR-194 | 58 |
| 1114 | GCCAATATTTCTGTGC | hsa-miR-195 | 51.7 |
| 1115 | GCCAATATTTCTGTGCT | hsa-miR-195 | 53.9 |
| 1116 | GAAAGAGACCGGTT | hsa-miR-128b | 50.1 |
| 1117 | GAAAGAGACCGGTTC | hsa-miR-128b | 53.3 |
| 1118 | GAAAGAGACCGGTTCA | hsa-miR-128b | 56.2 |
| 1119 | ATCTGCACTGTCAGC | hsa-miR-106b | 51.9 |
| 1120 | ATCTGCACTGTCAGCAC | hsa-miR-106b | 57.5 |
| 1121 | ATCTGCACTGTCAGCACT | hsa-miR-106b | 59.4 |
| 1122 | ACCGATTTCAAATGGT | hsa-miR-29c | 51.4 |
| 1123 | ACCGATTTCAAATGGTG | hsa-miR-29c | 54.2 |
| 1124 | ACATCGTTACCAGACA | hsa-miR-200a | 52.2 |
| 1125 | ACATCGTTACCAGACAGTG | hsa-miR-200a | 59.3 |
| 1126 | TCACCAAAACATGGAA | hsa-miR-302a | 51.6 |
| 1127 | TCACCAAAACATGGAAG | hsa-miR-302a | 53.7 |
| 1128 | GCAATCAGCTAACTACA | hsa-miR-34c | 52.7 |
| 1129 | GCAATCAGCTAACTACACTG | hsa-miR-34c | 59.4 |
| 1130 | GCTTTGACAATACTATTG | hsa-miR-301 | 50.6 |
| 1131 | GCTTTGACAATACTATTGCAC | hsa-miR-301 | 59.2 |
| 1132 | ACAGGATTGAGGGGGG | hsa-miR-296 | 59.3 |
| 1133 | ATGCCCTTTCATCATTGC | hsa-miR-130b | 57.2 |
| 1134 | ATGCCCTTTCATCATTGCA | hsa-miR-130b | 59.6 |
| 1135 | GCTGTAAACATCCGA | hsa-miR-30e-3p | 51.5 |
| 1136 | TCCAGTCAAGGATGT | hsa-miR-30e-5p | 50.1 |
| 1137 | TCCAGTCAAGGATGTT | hsa-miR-30e-5p | 52.2 |
| 1138 | TCCAGTCAAGGATGTTT | hsa-miR-30e-5p | 54.1 |
| 1139 | TCCAGTCAAGGATGTTTA | hsa-miR-30e-5p | 54.7 |
| 1140 | TCCAGTCAAGGATGTTTAC | hsa-miR-30e-5p | 57.1 |
| 1141 | GTACCCCTGGAGATTC | hsa-miR-361 | 55.3 |
| 1142 | GTACCCCTGGAGATTCT | hsa-miR-361 | 57.5 |
| 1143 | CTACTAAAACATGGAAGC | hsa-miR-302b | 53.5 |
| 1144 | CTACTAAAACATGGAAGCA | hsa-miR-302b | 55.9 |
| 1145 | CAGCAGGTACCCCCA | hsa-miR-302c* | 60 |
| 1146 | ACACTCAAACATGGAA | hsa-miR-302d | 50.5 |
| 1147 | ACACTCAAACATGGAAG | hsa-miR-302d | 52.8 |
| 1148 | ACACTCAAACATGGAAGC | hsa-miR-302d | 57.1 |
| 1149 | TCACCATTGCTAAAGTG | hsa-miR-367 | 52.6 |
| 1150 | AAACGTGGAATTTCCT | hsa-miR-368 | 51.6 |
| 1151 | AAACGTGGAATTTCCTC | hsa-miR-368 | 54.3 |
| 1152 | AAACGTGGAATTTCCTCT | hsa-miR-368 | 56.2 |
| 1153 | AAACGTGGAATTTCCTCTA | hsa-miR-368 | 56.7 |
| 1154 | AAACGTGGAATTTCCTCTAT | hsa-miR-368 | 57.4 |
| 1155 | ACACCCCAAAATCGA | hsa-miR-373 | 52.7 |
| 1156 | GGAAAGCGCCCCCA | hsa-miR-373* | 61.3 |
| 1157 | CACTTATCAGGTTGTATT | hsa-miR-374 | 51.5 |
| 1158 | CACTTATCAGGTTGTATTA | hsa-miR-374 | 52.2 |
| 1159 | CACTTATCAGGTTGTATTAT | hsa-miR-374 | 53 |
| 1160 | CACTTATCAGGTTGTATTATA | hsa-miR-374 | 53.6 |
| 1161 | ACGTGGATTTTCCTC | hsa-miR-376a | 50.6 |
| 1162 | ACGTGGATTTTCCTCT | hsa-miR-376a | 52.9 |
| 1163 | ACGTGGATTTTCCTCTA | hsa-miR-376a | 53.6 |
| 1164 | ACGTGGATTTTCCTCTAT | hsa-miR-376a | 54.5 |
| 1165 | ACAAAAGTTGCCTTTG | hsa-miR-377 | 50.8 |
| 1166 | ACAAAAGTTGCCTTTGT | hsa-miR-377 | 53.5 |
| 1167 | ACACAGGACCTGGA | hsa-miR-378 | 51.6 |
| 1168 | ACACAGGACCTGGAG | hsa-miR-378 | 54.2 |
| 1169 | ACACAGGACCTGGAGT | hsa-miR-378 | 57.2 |
| 1170 | GGCCTTCTGACTCC | hsa-miR-422b | 53.5 |
| 1171 | TACGTTCCATAGTCTAC | hsa-miR-379 | 50.2 |
| 1172 | AGCCACAATCACCTT | hsa-miR-383 | 51.2 |
| 1173 | AGCCACAATCACCTTC | hsa-miR-383 | 54.2 |
| 1174 | AGCCACAATCACCTTCT | hsa-miR-383 | 56.3 |
| 1175 | GGCTATAAAGTAACTGAG | hsa-miR-340 | 51.2 |
| 1176 | GGCTATAAAGTAACTGAGA | hsa-miR-340 | 53.8 |
| 1177 | ACGGAAGGGCAGAG | hsa-miR-328 | 54 |
| 1178 | ACGGAAGGGCAGAGA | hsa-miR-328 | 57 |
| 1179 | GACGGGTGCGATTT | hsa-miR-342 | 54.8 |
| 1180 | CCTCAAGGAGCTTC | hsa-miR-151 | 50.6 |
| 1181 | CCTCAAGGAGCTTCA | hsa-miR-151 | 53.9 |
| 1182 | ACAAAGTTCTGTGATGC | hsa-miR-148b | 53.3 |
| 1183 | ACAAAGTTCTGTGATGCA | hsa-miR-148b | 55.9 |
| 1184 | TTCTAGGATAGGCCCA | hsa-miR-331 | 52.9 |
| 1185 | TTCTAGGATAGGCCCAG | hsa-miR-331 | 55.2 |
| 1186 | ACACCAATGCCCTAG | hsa-miR-324-5p | 51.5 |
| 1187 | TCAACAAAATCACTGATG | hsa-miR-338 | 52.1 |
| 1188 | TCAACAAAATCACTGATGC | hsa-miR-338 | 56.2 |
| 1189 | TCAACAAAATCACTGATGCT | hsa-miR-338 | 57.9 |
| 1190 | TGAGCTCCTGGAGG | hsa-miR-339 | 52.3 |
| 1191 | TGAGCTCCTGGAGGA | hsa-miR-339 | 55.6 |
| 1192 | ACATTTTTCGTTATTGCT | hsa-miR-335 | 51.7 |
| 1193 | TAGCTGGTTGAAGGG | hsa-miR-133b | 51.7 |
| 1194 | TAGCTGGTTGAAGGGG | hsa-miR-133b | 56.2 |
| 1195 | GCCCTGGACTAGGA | hsa-miR-345 | 53.8 |
| 1196 | GGCCTTCTGACCCT | hsa-miR-422a | 55.2 |
| 1197 | GGCCTTCTGACCCTA | hsa-miR-422a | 55.8 |
| 1198 | GGCCTTCTGACCCTAA | hsa-miR-422a | 57.6 |
| 1199 | CTGAGGGGCCTCAGA | hsa-miR-423 | 59.4 |
| 1200 | TTCAAAACATGAATTGC | hsa-miR-424 | 50 |
| 1201 | TTCAAAACATGAATTGCT | hsa-miR-424 | 52.1 |
| 1202 | TTCAAAACATGAATTGCTG | hsa-miR-424 | 54.5 |
| 1203 | TTCAAAACATGAATTGCTGC | hsa-miR-424 | 58.2 |
| 1204 | TGAGACACACTTTGC | dme-miR-3 | 50.5 |
| 1205 | TGAGACACACTTTGCC | dme-miR-3 | 54.8 |
| 1206 | ACTATACAACCTACTACCT | dme-let-7 | 52 |
| 1207 | ACTATACAACCTACTACCTC | dme-let-7 | 54.5 |
| 1208 | ACTATACAACCTACTACCTCA | dme-let-7 | 56.9 |
| 1209 | TAGGAGAGAGAAAAAGAC | dme-miR-14 | 52.3 |
| 1210 | TAGGAGAGAGAAAAAGACT | dme-miR-14 | 54.3 |
| 1211 | TAGGAGAGAGAAAAAGACTG | dme-miR-14 | 56.7 |
| 1212 | TAGGAGAGAGAAAAAGACTGA | dme-miR-14 | 58.9 |
| 1213 | TCAGCTATGCCGAC | dme-miR-31a | 50.5 |
| 1214 | TCAGCTATGCCGACA | dme-miR-31a | 53.7 |
| 1215 | TCAGCTATGCCGACAT | dme-miR-31a | 54.6 |
| 1216 | TCAGCTATGCCGACATC | dme-miR-31a | 57.2 |
| 1217 | GCTCCTCAAAGCTG | dme-miR-2b | 50.7 |
| 1218 | AAAAAGAACAGCCACT | dme-miR-6 | 51 |
| 1219 | AAAAAGAACAGCCACTG | dme-miR-6 | 53.9 |
| 1220 | AAAAAGAACAGCCACTGT | dme-miR-6 | 56.4 |
| 1221 | CGGGGCGAGAGAAT | dme-miR-184* | 56.7 |
| 1222 | CGGGGCGAGAGAATG | dme-miR-184* | 59.5 |
| 1223 | GCACTGATTTCGAATG | dme-miR-285 | 52.3 |
| 1224 | GCACTGATTTCGAATGG | dme-miR-285 | 56.4 |
| 1225 | CTCACAGTATAATCCTGT | dme-miR-308 | 52.8 |
| 1226 | CTCACAGTATAATCCTGTG | dme-miR-308 | 55.4 |
| 1227 | CTCACAGTATAATCCTGTGA | dme-miR-308 | 57.6 |
| 1228 | CTCACAGTATAATCCTGTGAT | dme-miR-308 | 58.3 |
| 1229 | CGCCAGTAAGCGGA | dme-miR-316 | 57 |
| 1230 | CGCCAGTAAGCGGAA | dme-miR-316 | 58.6 |
| 1231 | GCTCATCAAAGCTGG | dme-miR-2a | 52.6 |
| 1232 | GCTCATCAAAGCTGGC | dme-miR-2a | 57.5 |
| 1233 | GCTCATCAAAGCTGGCT | dme-miR-2a | 59.4 |
| 1234 | GCCCATCAAAGCTG | dme-miR-2c | 51.6 |
| 1235 | GCCCATCAAAGCTGG | dme-miR-2c | 56.3 |
| 1236 | CAGCTATTCCGACAT | dme-miR-31b | 50.5 |
| 1237 | CAGCTATTCCGACATC | dme-miR-31b | 53.5 |
| 1238 | CAGCTATTCCGACATCT | dme-miR-31b | 55.6 |
| 1239 | CAGCTATTCCGACATCTT | dme-miR-31b | 57.1 |
| 1240 | CAGCTATTCCGACATCTTG | dme-miR-31b | 59.4 |

In particular embodiments, each probe includes a target-complementary sequence selected from SEQ ID NOs: 1-1240. SEQ ID NOS: 1-1240 includes sequences selected based on known miRNA sequences, which are given SEQ ID NOS: 1268-1581 (and are listed in Table 4, infra). In certain embodiments, the target-complementary sequence of each probe is independently selected from SEQ ID NOS: 916-1240. In particular embodiments, a probe having a target-complementary sequence selected from SEQ ID NOS: 1241 to 1250 may be employed, particulary in addition to probes having target-complementary sequences selected from SEQ ID NOs: 1-1240, or selected from SEQ ID NOS: 916-1240. In particular embodiments a probe having a target-complementary sequence selected from SEQ ID NOS: 1241 to 1250 may be employed in addition to probes having target-complementary sequences selected from SEQ ID NOS: 290-296.

In particular embodiments, a subject probe may includes a Tm enhancement domain, a linker, or both. Some examples are shown in Table 2, which list some example target-complementary sequences (SEQ ID NOS: 7-14, list from 5′ to 3′) along with the same sequences having a linker and Tm enhancement domain. By inspection of the sequences listed in Table 2, it be apparent that the sequences of SED ID NOS: 1252-1259 include the sequences of SEQ ID NOS: 7-14, respectively, plus a single base “G” (a nucleotide clamp) at the 5′ end and a T(10) linker at the 3′ end. The sequences of SEQ ID NOS: 1260-1267 includes the sequences of SEQ ID NOS: 1252-1259 plus a further hairpin Tm enhancement domain having a sequence of CGCTCGGGTTTTCCCGAGCG (SEQ ID NO: 1251). A subject probe includes a target-complementary sequence, plus an optional linker and/or an optional Tm enhancement domain; examples of subject probes having target-complementary sequences selected from SEQ ID NOS: 7-14 are given in Table 2.

TABLE 2

| | | |
|---|---|---|
| SEQ ID | | Target |
| NO: | Sequence | miRNA |

| | | |
|---|---|---|
| 7 | AACTATACAACCTACTACC | hsa-let-7a |
| 1252 | GAACTATACAACCTACTACCTTTTTTTTTT | hsa-let-7a |
| 1260 | CGCTCGGGTTTTCCCGAGCGGAACTATACA | hsa-let-7a |
| | ACCTACTACCTTTTTTTTTT | |
| 8 | AACTATACAACCTACTACCT | hsa-let-7a |
| 1253 | GAACTATACAACCTACTACCTTTTTTTTTTT | hsa-let-7a |
| 1261 | CGCTCGGGTTTTCCCGAGCGGAACTATACA | hsa-let-7a |
| | ACCTACTACCTTTTTTTTTTT | |
| 9 | AACCACACAACCTACTA | hsa-let-7b |
| 1254 | GAACCACACAACCTACTATTTTTTTTTT | hsa-let-7b |
| 1262 | CGCTCGGGTTTTCCCGAGCGGAACCACACA | hsa-let-7b |
| | ACCTACTATTTTTTTTTT | |
| 10 | AACCACACAACCTACTAC | hsa-let-7b |
| 1255 | GAACCACACAACCTACTACTTTTTTTTTT | hsa-let-7b |
| 1263 | CGCTCGGGTTTTCCCGAGCGGAACCACACA | hsa-let-7b |
| | ACCTACTACTTTTTTTTTT | |
| 11 | AACCATACAACCTACTAC | hsa-let-7c |
| 1256 | GAACCATACAACCTACTACTTTTTTTTTT | hsa-let-7c |
| 1264 | CGCTCGGGTTTTCCCGAGCGGAACCATACA | hsa-let-7c |
| | ACCTACTACTTTTTTTTTT | |
| 12 | AACCATACAACCTACTACC | hsa-let-7c |
| 1257 | GAACCATACAACCTACTACCTTTTTTTTTT | hsa-let-7c |
| 1265 | CGCTCGGGTTTTCCCGAGCGGAACCATACA | hsa-let-7c |
| | ACCTACTACCTTTTTTTTTT | |
| 13 | AACCATACAACCTACTACCT | hsa-let-7c |
| 1258 | GAACCATACAACCTACTACCTTTTTTTTTTT | hsa-let-7c |
| 1266 | CGCTCGGGTTTTCCCGAGCGGAACCATACA | hsa-let-7c |
| | ACCTACTACCTTTTTTTTTTT | |
| 14 | ACTATGCAACCTACTACCT | hsa-let-7d |
| 1259 | GACTATGCAACCTACTACCTTTTTTTTTTT | hsa-let-7d |
| 1267 | CGCTCGGGTTTTCCCGAGCGGACTATGCAA | hsa-let-7d |
| | CCTACTACCTTTTTTTTTTT | |

Tm Enhancement Domain:

As noted above, a subject probe generally contains a target-complementary sequence 104 that base-pairs with a target miRNA to form a probe/target duplex. In particular embodiments the probe includes a Tm enhancement domain 106 that increases stability of the probe/target duplex. Tm enhancement domain 106 may increase probe/target duplex stability via a number of mechanisms, including, for example, by providing a nucleotide clamp 106a (as illustrated in FIG. 2A) to which an extended polynucleotide, e.g., extended miRNA, may bind, or by providing a hairpin structure 106b (as illustrated in FIG. 2B) that increases stability via coaxial stacking. In certain embodiments and as illustrated in FIG. 2C, Tm enhancement domain 106 may contain both a nucleotide clamp 106a and a hairpin structure 106b. The sequence of the Tm enhancement domain 106 is generally unrelated to the sequence of the target-complementary sequence 104. Further description of such Tm enhancement domains is provided in copending U.S. patent application Ser. No. 11/173693 filed on Jul. 1, 2005 by Wang.

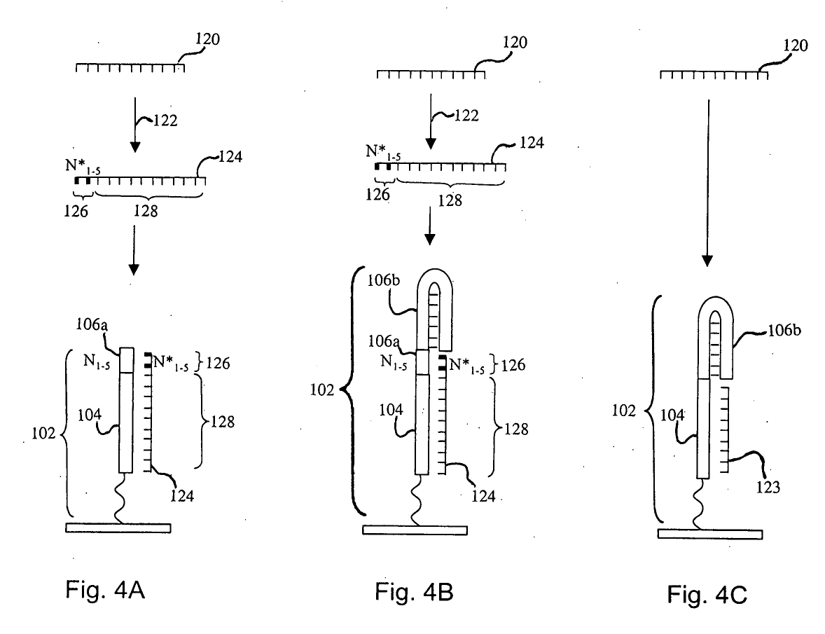

As mentioned above and as illustrated in FIG. 2A, Tm enhancement domain 106 is immediately adjacent to target-complementary sequence 104 and may contain a nucleotide clamp 106a, where a nucleotide clamp 106a contains a contiguous sequence of up to about 5 nucleotides (i.e., 1, 2, 3, 4 or 5 nucleotides). The identity of the nucleotides employed in the nucleotide clamp may be the same as each other or different from each other. As illustrated in FIG. 2A, a nucleotide clamp contains N₁₋₅, wherein “N” is any nucleotide, particularly a G or a C, possibly an A, T, or U, or a modified base. In certain embodiments, illustrated in FIG. 4A, a subject probe 102 containing a nucleotide clamp 106a is employed in a method in which the miRNA 120 to be detected by the probe 102 is extended 122, i.e. has additional nucleotide(s) added to the miRNA (in certain embodiments during labeling of the miRNA) to produce an extended miRNA 124. The extended miRNA includes an extended portion 126 having contiguous sequence of up to about 5 nucleotides (i.e., 1, 2, 3, 4 or 5 nucleotides) (illustrated in FIG. 4A as N*₁₋₅), wherein extended portion 126 typically is complementary to the nucleotide clamp 106a (i.e. N*₁₋₅ illustrates a contiguous sequence of up to about 5 nucleotides that is complementary to N₁₋₅). In use, the miRNA, e.g. the extended miRNA, is contacted 125 with the probe under conditions sufficient to provide for specific binding of the probe to the miRNA to form a probe/target duplex. In the probe/target duplex formed between a probe 102 containing a nucleotide clamp 106a and an extended miRNA 124, the extended portion 126 of the extended miRNA 124 base-pairs with the nucleotide clamp 106a of the probe 102 and the non-extended miRNA sequence 128 base pairs with target-complementary sequence 104. In particular embodiments of particular probes, nucleotide clamp 106a (having nucleotides designated N₁₋₅) may be shorter than the extended portion 126 or the miRNA; e.g. the probe may be designed in this way to reduce the base pairing of the extended miRNA 124 to the nucleotide clamp 106a, for example to obtain a set of probes that are characterized as having similar melting temperatures (Tm).

Also as mentioned above and as illustrated in FIG. 2B, Tm enhancement domain 106 is immediately adjacent to target-complementary sequence 104 and may contain a hairpin structure 106b, where a hairpin structure 106b has a loop 112 of at least 3 or 4 nucleotides (up to about 8 or 10 nucleotides) and a double-stranded stem 114 (of about 6 to about 20 base pairs) in which complementary nucleotides bind to each other in an anti-parallel manner. The hairpin structure 106b may contain from approximately 5 to about 45 nucleotides, e.g., about 8 to about 30 nucleotides. As shown in FIG. 4B and FIG. 4C, when miRNA 124, 123 is bound to a probe 102 containing a hairpin region 106b, a terminal nucleotide of the miRNA generally occupies a position that is immediately adjacent to a terminal nucleotide of the probe 102. In effect, in this embodiment, the probe/target duplex produced by binding of a miRNA 124, 123 to a probe 102 resembles a long hairpin structure containing a nick in the stem of the hairpin. The description of the embodiments of FIG. 4B and FIG. 4C is essentially the same as that given for the embodiment of FIG. 4A, with the following considerations. The embodiment illustrated in FIG. 4B shows a method in which the miRNA 120 to be detected by the probe 102 is extended 122 (in certain embodiments during labeling of the miRNA) to produce an extended miRNA 124, which is then hybridized to the probe 102. The embodiment illustrated in FIG. 4C shows a method in which the miRNA 120 to be detected by the probe 102 is not extended 122, and the un-extended miRNA is then hybridized to the probe 102. The hairpin structure may assist in increasing probe specificity by preferentially binding to miRNAs (having known length and sequence), rather than pre-miRNAs or pri-miRNAs (i.e., precursor RNAs that are cleaved to produce miRNAs) in a sample. The presence of hairpin structure, in certain embodiments, allows a probe to discriminate between a miRNA and a precursor of that miRNA that is present in same sample. Also, the extra stabilization provided by helical stacking is present only when the target miRNA is the proper length.

Linker:

As noted above, a probe may include a linker that is bound to the target-complementary sequence. In embodiments in which the probe is bound to a surface of an array support, the target-complementary sequence is bound to the array support via the linker. The linker sequence may be any sequence that does not substantially interfere with hybridization of targets to probes, e.g. the sequence of the probe should be selected to not be complementary to any analytes expected to be assayed. An example used herein is a (T)₁₀ linker (ten contiguous Ts), wherein one end of the (T)₁₀ linker is bound to the target-complementary sequence, and the probe is bound to the array surface via the other end of the (T)₁₀ linker. Thus, the sequence and length of the linker can be varied (such as 0-20 nucleotides).

Probe Set:

In particular embodiments, a probe set comprising subject probes is provided. In particular embodiments, a probe set includes at least five subject probes, wherein each of said at least five subject probes has a target-complementary sequence independently selected from SEQ ID NOs: 1-1240. In some embodiments, a probe set includes at least 10 subject probes, at least 20 subject probes, at least 50 subject probes, at least 100 subject probes, at least 200 subject probes, or more subject probes, such as up to 1000 subject probes, up to 2000 subject probes, or even more subject probes, and each of the subject probes has a target-complementary sequence independently selected from SEQ ID NOs: 1-1240. In certain embodiments, the target-complementary sequence of each probe is independently selected from the group consisting of SEQ ID NOS: 916-1240. Each probe of the probe set may include a linker and/ or Tm enhancement domain, as described above.

In particular embodiments, the probe set further includes at least one probe having a target-complementary sequence selected from SEQ ID NOS: 1241 to 1250. SEQ ID NOS: 1241 to 1250 are directed to a few miRNAs, such as human miR-20a and miR-20b, in which sequence homologous miRNAs differ by one 5′ end nucleotide and one nucleotide in the middle of the miRNA sequences. In such cases, additional Tm matching probes are generated by successively removing 3′ nucleotides of the miRNA in the probe-target base pairing (i.e., by removing base pairing sequence from the 5′ end of the target-complementary sequence of the probe). In use, the results of hybridization experiments with these probes will be compared to the results with 5′ modified probes (probes with target complementary sequences selected from SEQ ID NOS:290-296). Table 3 compares the sequences of SEQ ID NOS: 1241 to 1250 (having successive nucleotides deleted from the 3′ end) with the sequences of SEQ ID NOS:290-296 (having successive nucleotides delected from the 5′ end).

TABLE 3

| | | | |
|---|---|---|---|
| SEQ | | | |
| ID | | Target | Tm |
| NO: | Sequence | miRNA | (calc) |

| | | | |
|---|---|---|---|
| 1241 | CTGCACTATAAGCACTTTA | hsa-miR-20a | 50.7 |
| 1242 | CCTGCACTATAAGCACTTTA | hsa-miR-20a | 54.5 |
| 1243 | ACCTGCACTATAAGCACTTTA | hsa-miR-20a | 56.8 |
| 1244 | TACCTGCACTATAAGCACTTTA | hsa-miR-20a | 57.2 |
| 1245 | CTACCTGCACTATAAGCACTTTA | hsa-miR-20a | 58.8 |
| 290 | CTACCTGCACTATAAGC | hsa-miR-20a | 52.9 |
| 291 | CTACCTGCACTATAAGCA | hsa-miR-20a | 55.6 |
| 292 | CTACCTGCACTATAAGCAC | hsa-miR-20a | 57.9 |
| 293 | CTACCTGCACTATAAGCACT | hsa-miR-20a | 59.6 |
| 1377 | UAAAGUGCUUAUAGUGCAGGUAG | (hsa-miR-20a) | |
| 1246 | CTGCACTATGAGCACTTTG | hsa-miR-20b | 55.6 |
| 1247 | CCTGCACTATGAGCACTTTG | hsa-miR-20b | 59.2 |
| 1248 | ACCTGCACTATGAGCACTTTG | hsa-miR-20b | 61.3 |
| 1249 | TACCTGCACTATGAGCACTTTG | hsa-miR-20b | 61.5 |
| 1250 | CTACCTGCACTATGAGCACTTTG | hsa-miR-20b | 62.9 |
| 294 | CTACCTGCACTATGAG | hsa-miR-20b | 51.2 |
| 295 | CTACCTGCACTATGAGC | hsa-miR-20b | 56.0 |
| 296 | CTACCTGCACTATGAGCA | hsa-miR-20b | 58.5 |
| 1378 | CAAAGUGCUCAUAGUGCAGGUAG | (hsa-miR-20b) | |

Note that the sequences of human miRNAs miR-20a and miR-20b are given in SEQ ID NOs: 1377 and 1378. Thus, in addition to any other probes in a probe set of the present invention, in certain embodiments the probe set may further include at least one probe with a target complementary sequence selected from SEQ ID NOS:290-296 and at least one corresponding probe with a target complementary sequence selected from SEQ ID NOS: 1241 or 1250. In this regard, the corresponding probe is directed to the same target miRNA that the probe with a target complementary sequence selected from SEQ ID NOS:290-296 is directed to.

Nested Set:

Referring now to Table 1, it can be seen that the sequences given in SEQ ID NOS:1-1250 include nested sets of sequences, in which a first (longer) sequence shares the same sequence of a second (shorter) sequence, but includes one or more additional bases. An example is SEQ ID NOS:33-37, in which the longest sequence (SEQ ID NO:37) comprises the same sequence of the shorter sequences plus 1, 2, 3, or 4 bases (relative to SEQ IDS 36, 35, 34, 33, respectively):

| | |
|---|---|
| 33 | ACAGGAGTCTGAGCA |
| 34 | ACAGGAGTCTGAGCAT |
| 35 | ACAGGAGTCTGAGCATT |
| 36 | ACAGGAGTCTGAGCATTT |
| 37 | ACAGGAGTCTGAGCATTTG |

As used herein, “nested set” references two or more sequences bearing such a relationship to each other. The “nested set” may include two sequences wherein the longer of the two sequences comprises that shorter of the two sequences plus 1, 2, 3, 4, or 5, or more bases. In certain embodiments, the nested set may include three or more sequences.

In particular embodiments, a subject probe set may include two, three, or more probes that are characterized as having target-complementary sequences that form a nested set. In other words, more than one member of a given nested set selected from the sequences of SEQ ID NOS: 1-1250 may be included in a subject probe set. In such embodiments, the target-complementary sequence of one probe of the probe set will be shorter or longer by about 1 to about 5 bases, compared to the target-complementary sequence of another probe of the probe set, but otherwise the two target-complementary sequences share the same “common” sequence. That is, one probe of the probe set will have target-complementary sequence that is shorter or longer by about, e.g. 1, 2, 3, 4, or 5 bases (but will otherwise share a common sequence), compared to the target-complementary sequence of another probe of the probe set, wherein both probes are directed to the same target miRNA. In some such embodiments, the base (or bases) that are omitted are typically those from the 3′-end of the target-complementary sequence (see, e.g. selected sequences in SEQ ID NOS: 1-1240). In certain embodiments, the base (or bases) that are omitted are typically those from the 5′-end of the target-complementary sequence (see, e.g. selected sequences in SEQ ID NOS: 1241-1250). In particular embodiments, the target-complementary sequence of a first probe of the probe set differs from the target-complementary sequence of a second probe of the probe set by lacking at least one base (e.g. lacking at least two bases, lacking at least three bases, lacking up to about five bases) relative to the target-complementary sequence of the second probe, wherein the first probe and second probe are directed to the same miRNA.

Similar Tm:

In particular embodiments, the probes of the probe set are selected such that the probe/target duplexes formed will have similar thermal stabilities. The melting temperature (‘Tm’) of the probe/target duplexes should be high enough to eliminate or reduce any non-specific binding (e.g. preventing non-complementary sequences from forming double-stranded structures). In such embodiments, the melting temperatures of at least 80% of the probe/target duplexes will be within about 15° C. of each other, typically within about 12° C. of each other, about 10° C. of each other, or about 5° C. of each other. In certain embodiments, the difference between the maximum and minimum melting temperatures is less than about 20° C., less than about 15° C., less than about 10C, or less than about 5° C. In some embodiments, probe sequences may be selected based on experimental determinations of the melting temperatures or calculations of the theoretical melting temperatures. In certain embodiments, putative probe sequences may first be selected based on calculations of their theoretical melting temperatures and then be confirmed experimentally. Methods for determining the melting temperature of nucleic acid duplexes are known in the art. See for example, Sambrook and Russell (2001) Molecular Cloning: A Laboratory Handbook, 10.38-10.41 and 10.47, which is incorporated by reference in its entirety.

A value for melting temperature can be determined mathematically using equations and algorithms known in the art. For duplex oligonucleotides shorter than 25 bp, “The Wallace Rule” can be used in which:

Tm (in ° C.)=2(A+T)+4(C+G), where

(A+T) —the sum of the A and T residues in the oligonucleotide,

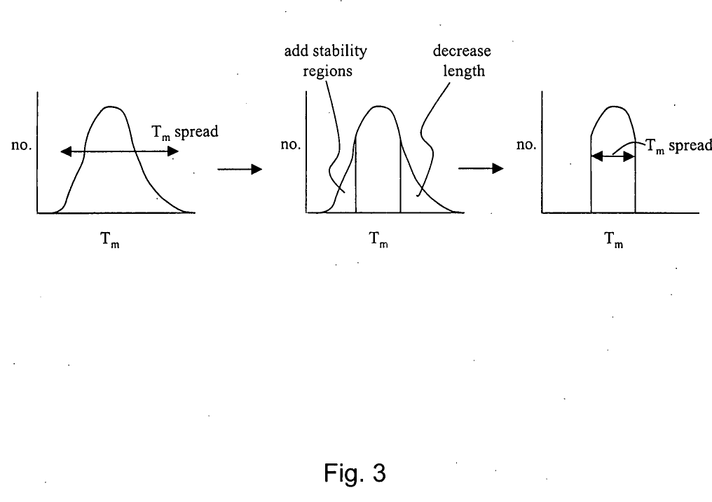

(C+G) —the sum of G and C residues in the oligonucleotide (see Wallace et al., Nucleic Acids Res. (1979) 6: 3543-3557). Computer programs for estimating Tm are also available (see, e.g., Le Novere, Bioinformatics (2001) 17(12): 1226-1227). VisualOmp (DNA Software, Inc., Ann Arbor, Mich.) is an example of commercially available software for calculating nucleic acid duplex melting temperature. As illustrated by the left hand graph of FIG. 3, the Tms of the set of probe sequences that are complementary to the polynucleotides (e.g., miRNAs) of a population of polynucleotides are distributed across a Tm spread (a Tm spread being difference in temperature between the highest and lowest Tm of the set). As illustrated in this graph, the Tms may have an approximate normal distribution and form an approximate bell-shaped curve when plotted as shown. As illustrated in the middle graph of FIG. 3, in designing probes for the polynucleotides, the length of the complementary sequences having a higher Tm is decreased (thereby decreasing the Tm of those sequences) and stability sequences are added to the complementary sequences having a lower Tm (thereby increasing the Tm of those sequences). As illustrated in the right-hand graph of FIG. 3, once the Tms of the population of complementary sequences have been adjusted by reducing the length of the sequences or by adding Tm enhancement domains, the spread of the Tms of the population is significantly reduced. Such a reduction in Tm spread is highly desirable in microarray analysis.

Arrays

In certain embodiments of the invention a subject probe is a “surface-bound probe”, where such a probe is bound, usually covalently but in certain embodiments non-covalently, to a surface of a solid substrate, e.g., a sheet, bead, or other structure. In certain embodiments, a surface-bound probe may be immobilized on a surface of a planar support, e.g., as part of an array.

A subject array may contain a plurality of features (e.g., 2 or more, about 5 or more, about 10 or more, about 15 or more, about 20 or more, about 30 or more, about 50 or more, about 100 or more, about 200 or more, about 500 or more, about 1000 or more, usually up to about 10,000 or about 20,000 or more features, etc.), each feature containing a capture agent capable of binding a target. In embodiments in accordance with the present invention, the array includes features wherein the capture agents of at least some of the features are probes directed to miRNAs as described above. In certain embodiments, the array may also include features wherein the capture agent of each feature is not directed to a miRNA, e.g. the target of the capture agent is some other target, e.g. MRNA, other small RNA, or other polynucleotide, or the capture agent is a ‘control’. In particular embodiments the array has at least five different subject probes attached to the array support, e.g. each of the subject probes present at a separate feature of the array. In some embodiments, at least 10, at least 20, at least 40, at least 100, or at least 200 different subject probes are attached to the array support, e.g. each of the subject probes present at a separate feature of the array. In certain embodiments, at least 5%, at least 10%, at least 20%, or at least 40% of the features of an array contain a subject probe. As few as one and as many as all of the subject probes of a subject array may contain a Tm enhancement domain. In certain embodiments, at least 5%, at least 10% or at least 20% of the subject probes of an array contain a Tm enhancement domain.

Typically, different probes are present in different features of an array, i.e., each spatially addressable area of an array is associated with a different probe. In many embodiments a single type of probe is present in each feature (i.e., each individual feature contains one sequence of probe). However, in certain embodiments, the nucleic acids in a feature may be a mixture of nucleic acids having different sequences, e.g. two or more different probes may be co-located at the same feature.

A subject array typically may have at least five different subject probes, i.e. the array includes a probe set having at least five different subject probes. However, in certain embodiments, a subject array may include a probe set having at least 10, at least 20, at least 50, at least 100, or at least 200 subject probes that are directed to (i.e., may be used to detect) a corresponding number of miRNAs. In particular embodiments, the subject arrays may include probes for detecting at least a portion or all of the identified miRNAs of a particular organism, e.g. human.

In general, methods for the preparation of nucleic acid arrays, particularly oligonucleotide arrays, are well known in the art (see, e.g., Harrington et al,. Curr Opin Microbiol. (2000) 3:285-91, and Lipshutz et al., Nat Genet. (1999) 21:20-4) and need not be described in any great detail. The subject arrays can be fabricated using any means available, including drop deposition from pulse jets or from fluid-filled tips, etc., or using photolithographic means. Either polynucleotide precursor units (such as nucleotide monomers), in the case of in situ fabrication, or previously synthesized polynucleotides can be deposited. Such methods are described in detail in, for example U.S. Pat. Nos. 6,242,266, 6,232,072, 6,180,351, 6,171,797, 6,323,043, etc., the disclosures of which are herein incorporated by reference.

In certain embodiments, an array of the invention may contain probes that all have a similar Tm. The spread of Tms of such arrays may be less than about 10° C., less than about 5° C., or less than about 2° C., for example. The spread of Tms of an array may be theoretically determined, or, in certain embodiments, experimentally determined.

Methods for Assessing miRNA in a Sample

The subject invention provides a method of analyzing a sample for miRNA, e.g. assessing for the presence or amount of a miRNA. In general, the method includes the following steps: a) contacting the sample with a array comprising a subject probe set (such as described above) under conditions sufficient for specific binding to occur; and b) interrogating the array to obtain information about miRNAs in the sample. Interrogating the array typically involves detecting the presence of any detectable label associated with the probes of the probe set on the array, thereby evaluating the amount of the respective target miRNAs in the sample.

In embodiments in which a probe containing a nucleotide clamp is employed, miRNAs in a sample containing the miRNAs may be extended to add nucleotides that are complementary to the nucleotide clamp of the nucleic acid probe. The addition of the nucleotides to the miRNAs may be done before, simultaneously with, or after labeling. In representative embodiments, a mononucleotide, di-nucleotide, tri-nucleotide, tetra-nucleotide or penta-nucleotide moiety is added to either the 3′ or the 5′ ends of the miRNAs (e.g. sample comprising isolated miRNAs) using an enzyme, e.g., an RNA or DNA ligase or terminal transferase. A variety of RNA and DNA ligases may be purchased from a variety, of vendors (e.g., Pharmacia, Piscataway, NJ, New England Biolabs, Berverly MA, and Roche Diagnostics, Indianapolis, IN) and employed according to the instructions supplied therewith. In an embodiment of particular interest, the nucleotide(s) added to the miRNA are covalently linked to a label, e.g., a fluorophore, such that the miRNA is labeled by the addition of the nucleotide label moiety. Labeled mononucleotides, di-nucleotides, tri-nucleotides, tetra-nucleotides, penta-nucleotides or higher order labeled polynucleotides are termed “nucleotide label moieties” herein. Further description of such Tm enhancement domains is provided in copending U.S. patent application Ser. No. 11/048225 filed on Jan. 31, 2005 by Wang entitled “RNA Labeling Method.”

For example, and as illustrated in FIG. 4A, nucleotides (N*₁₋₅) complementary to the nucleotide clamp of the probe are ligated to a terminus of a miRNA to produce an extended miRNA. The extended miRNA hybridizes to a nucleic acid probe containing a nucleotide clamp (N₁₋₅). The added nucleotides of the extended miRNA base pair with the clamp of the probe whereas the remainder of the extended miRNA base pair with the target-complementary sequence of the probe. As illustrated in FIG. 4B, nucleotides (N*₁₋₅) complementary to the nucleotide clamp of the probe are ligated to a terminus of a miRNA to produce an extended miRNA. The extended miRNA hybridizes to a probe containing a nucleotide clamp (N₁₋₅) and a hairpin. The added nucleotides of the extended miRNA base pair with the clamp of the probe whereas the remainder of the extended miRNA base pairs with the target-complementary sequence of the probe. The coaxial stacking and the nucleotide clamping increase the stability of the probe/target duplex. Finally and with reference to FIG. 4C, an miRNA hybridizes to a probe containing a hairpin structure. The miRNA base pairs with the target-complementary sequence of the probe and coaxial stacking increases the stability of the probe/target duplex.

In certain embodiments a subject array is employed to assess a sample of microRNAs that is prepared from a cell. Methods for preparing samples of miRNAs from cells are well known in the art (see, e.g., Lagos-Quintana et al, Science 294:853-858(2001); Grad et al, Mol Cell 11: 1253-1263 (2003); Mourelatos et al, Genes Dev 16:720-728(2002); Lagos-Quintana et al, Curr Biol 12:735-739(2002); Lagos-Quintana et al, RNA 9:175-179(2003) and other references cited above).

The sample is usually labeled to make a population of labeled miRNAs. In general, a sample may be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.), and, accordingly, such methods do not need to be described here in great detail. In particular embodiments, the sample is usually labeled with fluorescent label, which labels will be described in greater detail below.

Fluorescent dyes of particular interest include: xanthene dyes, e.g. fluorescein and rhodamine dyes, such as fluorescein isothiocyanate (FITC), 6 carboxyfluorescein (commonly known by the abbreviations FAM and F), 6 carboxy-2′,4′,7′,4,7-hexachlorofluorescein (HEX), 6 carboxy 4′, 5′ dichloro 2′, 7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarins, e.g. umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc.

In some embodiments, after labeling the labeled sample is contacted with a subject probe (e.g. a member of a subject probe set, e.g. of an array comprising the probe set) under stringent hybridization conditions, and any binding of labeled miRNA to a probe is detected by detecting the label associated with the probe.

In certain embodiments, binding of labeled miRNAs in the labeled sample is assessed with respect to binding of at least one control labeled sample. In one example, a suitable control labeled sample may be made from a control cell population, as will be described in greater detail below.

In certain embodiments, a sample and a control sample may be prepared and labeled, and relative binding of the labeled miRNAs in the samples to a subject probe may be assessed. Since the subject probe may be a surface-bound probe that is present at a feature of an array, in many embodiments, the samples are labeled and contacted with at least one array containing a subject probe, under stringent hybridization conditions.

In practicing the subject methods, the samples may be labeled to provide at least two different populations of labeled miRNAs that are to be compared. The populations of miRNAs may be labeled with the same label or different labels, depending on the actual assay protocol employed. For example, where each population is to be contacted with different but identical arrays, each population of miRNAs may be labeled with the same label. Alternatively, where both populations are to be simultaneously contacted with a single array of surface-bound probes, i.e., co-hybridized to the same array of immobilized probes, the two different populations are generally distinguishably labeled with respect to each other.

The samples are sometimes labeled using “distinguishable” labels in that the labels that can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Bostan Mass.) and POPRO3 and TOPRO3 (Molecular Probes, Eugene, Oreg.). Further suitable distinguishable detectable labels may be described in Kricka et al. (Ann. Clin. Biochem. 39:114-29, 2002).

Accordingly, in certain embodiments, at least a first population of miRNAs and a second population of miRNAs are produced from two different miRNA-containing samples, e.g., two populations of cells. As indicated above, depending on the particular assay protocol (e.g., whether both populations are to be hybridized simultaneously to a single array or whether each population is to be hybridized to two different but substantially identical, if not identical, arrays) the populations may be labeled with the same or different labels. As such, a feature of certain embodiments is that the different populations of miRNAs are labeled with the same label such that they are not distinguishably labeled. In yet other embodiments, a feature of the different populations of labeled nucleic acids is that the first and second labels are distinguishable from each other.

Generally, the subject methods comprise the following major steps: (1) provision of an array containing surface-bound subject probes; (2) hybridization of a population of labeled miRNAs to the surface-bound probes under conditions sufficient to provide for specific binding, e.g. typically under stringent hybridization conditions; (3) post-hybridization washes to remove nucleic acids not bound in the hybridization; and (4) detection of the hybridized miRNAs. The reagents used in each of these steps and their conditions for use may vary depending on the particular application.

As indicated above, hybridization is carried out under suitable hybridization conditions, which may vary in stringency as desired, typical conditions are sufficient to produce probe/target complexes on an array surface between complementary binding members, i.e., between surface-bound subject probes and complementary labeled miRNAs in a sample. In certain embodiments, stringent hybridization conditions may be employed. Representative stringent hybridization conditions that may be employed in these embodiments are provided above.

Thus, after nucleic acid purification of labeled miRNAs from unincorporated label, the populations of labeled miRNAs are usually contacted with an array of surface-bound probes, as discussed above, under conditions such that nucleic acid hybridization to the surface-bound probes can occur, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C., for example.

The above hybridization step may include agitation of the surface-bound probes and the sample of labeled miRNAs, where the agitation may be accomplished using any convenient protocol, e.g., shaking, rotating, spinning, and the like.

Standard hybridization techniques (e.g. under conditions sufficient to provide for specific binding of target miRNAs in the sample to the probes on the array) are used to hybridize a sample to a nucleic acid array. Suitable methods are described in many references (e.g., Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol 7, pgs 43-65 (Plenum Press, New York 1985). See also U.S. Pat. Nos: 6,335,167; 6,197,501; 5,830,645; and 5,665,549; the disclosures of which are herein incorporated by reference. Hybridizing the sample to the array is typically performed under stringent hybridization conditions, as described herein and as known in the art. Selection of appropriate conditions, including temperature, salt concentration, polynucleotide concentration, time(duration) of hybridization, stringency of washing conditions, and the like will depend on experimental design, including source of sample, identity of capture agents, degree of complementarity expected, etc., and may be determined as a matter of routine experimentation for those of ordinary skill in the art.

Following hybridization, the array-surface bound polynucleotides are typically washed to remove unbound nucleic acids. Washing may be performed using any convenient washing protocol, where the washing conditions are typically stringent, as described above.

Following hybridization and washing, as described above, the hybridization of the target miRNAs to the probes is then detected using standard techniques of reading the array, i.e. the array is interrogated. Reading the resultant hybridized array may be accomplished by illuminating the array and reading the location and intensity of resulting fluorescence at each feature of the array to detect any binding complexes (e.g. probe/target duplexes) on the surface of the array. For example, a scanner may be used for this purpose that is similar to the AGILENT MICROARRAY SCANNER available from Agilent Technologies, Palo Alto, CA. Other suitable devices and methods are described in U.S. Pat. No. 6,756,202 and U.S. Pat. No. 6,406,849. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

By “normalization” is meant that data corresponding to the two populations of polynucleotides are globally normalized to each other, and/or normalized to data obtained from controls (e.g., internal controls produce data that are predicted to equal in value in all of the data groups). Normalization generally involves multiplying each numerical value for one data group by a value that allows the direct comparison of those amounts to amounts in a second data group. Several normalization strategies have been described (Quackenbush et al, Nat Genet. 32 Suppl:496-501, 2002, Bilban et al Curr Issues Mol Biol. 4:57-64, 2002, Finkelstein et al, Plant Mol Biol.48(1-2):119-31, 2002, and Hegde et al, Biotechniques. 29:548-554, 2000). Specific examples of normalization suitable for use in the subject methods include linear normalization methods, non-linear normalization methods, e.g., using lowest local regression to paired data as a function of signal intensity, signal-dependent non-linear normalization, qspline normalization and spatial normalization, as described in Workman et al., (Genome Biol. 2002 3, 1-16). In certain embodiments, the numerical value associated with a feature signal is converted into a log number, either before or after normalization occurs. Data may be normalized to data obtained using a support-bound polynucleotide probe for a polynucleotide of known concentration, for example.

In certain embodiments, results from interrogating the array are used to assess the level of binding of the population of miRNAs from the sample to subject probes on the array. The term “level of binding” means any assessment of binding (e.g. a quantitative or qualitative, relative or absolute assessment), usually done, as is known in the art, by detecting signal (i.e., pixel brightness) from a label associated with the sample miRNA, e.g. the sample is labeled. The level of binding of labeled miRNA to probe is typically obtained by measuring the surface density of the bound label (or of a signal resulting from the label).

Accordingly, since the arrays used in the subject assays may contain probes for a plurality of different miRNAs, the presence of a plurality of different miRNAs in a sample may be assessed. The subject methods are therefore suitable for simultaneous assessment of a plurality of miRNAs in a sample.

In certain embodiments, a surface-bound probe may be assessed by evaluating its binding to two populations of nucleic acids that are distinguishably labeled. In these embodiments, for a single surface-bound probe of interest, the results obtained from hybridization with a first population of labeled nucleic acids may be compared to results obtained from hybridization with the second population of nucleic acids, usually after normalization of the data. The results may be expressed using any convenient means, e.g., as a number or numerical ratio, etc.

In typical embodiments of methods in accordance with the present invention, an isolated RNA sample may be labeled, e.g. with Cy5 or Cy3, and hybridized onto an array as follows: The labeled RNA is desalted (e.g. with BioRad MICRO BIO-SPIN™6 columns, as directed by BioRad instructions) to remove excess observable label remaining from the labeling reaction. The desalted sample of RNA is added to solution containing water and carrier (25-mer DNA with random sequence). The resulting solution is heated at about 100° C. for approximately 1 minute per 10 microliters of solution, and then immediately cooled on ice. The cooled solution is then added to hybridization buffer and mixed carefully. The final solution is then contacted with the array, e.g. in a SUREHYB hybridization chamber (Agilent Part Number:G2534A), and placed on rotisserie of hybridization oven overnight. The hybridization temperature is typically in the range from about 50° C. to about 60° C., or in the range from about 55° C. to about 60° C., although temperatures outside this range (e.g. in the range from about 30° C. to about 65° C., or in the range from about 45° C. to about 65° C.) may be used depending on the other experimental parameters, e.g. hybridization buffer composition and wash conditions. After the hybridization is complete, the array is washed thoroughly and dried with nitrogen as needed. The array is scanned (e.g. with an Agilent Scanner, Agilent Product Number: G2565BA). The data is then evaluated (e.g. using Agilent Feature Extraction Software, Agilent Product Number: G2567AA) for hybridization efficiency and specificity. Data may be further analyzed, e.g. using Spotfire software and Microsoft Excel.

Also provided by the subject invention are kits for practicing the subject methods, as described above. The subject kits include at least a probe set, as described above. For example, a kit may include an array support having the probe set attached to the surface of the array support. In certain embodiments the subject kits may also include reagents for isolating RNA from a source to provide an isolated sample of RNA. In some embodiments the subject kits optionally also include one or more constituents selected from reagents for labeling RNA, reagents for contacting the sample of RNA with the probe set (e.g., enzymes for use with the subject methods such as described above, control samples, reagents for performing an array hybridization, combinations thereof, etc.) The various components of the kit may be present in separate containers or certain compatible components may be precombined into a single container, as desired.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a suitable material, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable material.

The practice of the present invention will employ, unless otherwise indicated, conventional techniques of synthetic organic chemistry, biochemistry, molecular biology, and the like, which are within the skill of the art. Such techniques are explained fully in the literature. Unless otherwise defined herein, all technical and scientific terms used herein have the same meaning as commonly understood by one of ordinary skill in the art to which the invention belongs. The description herein is set forth so as to provide those of ordinary skill in the art with a complete disclosure and description of how to perform the methods and use compositions disclosed and claimed herein. Efforts have been made to ensure accuracy with respect to numbers (e.g., amounts, temperature, etc.) but some errors and deviation should be accounted for. Unless indicated otherwise, parts are parts by weight, temperature is in ° C and pressure is at or near atmospheric. Standard temperature and pressure are defined as 20° C. and 1 atmosphere. Some known sequences of miRNA are listed in Table 4.

TABLE 4

| SEQ ID | | |
|---|---|---|
| NO: | Sequence | Name |
| 1268 | UGAGGUAGUAGGUUGUAUAGUU | hsa-let-7a |
| 1269 | UGAGGUAGUAGGUUGUGUGGUU | hsa-let-7b |
| 1270 | UGAGGUAGUAGGUUGUAUGGUU | hsa-let-7c |
| 1271 | AGAGGUAGUAGGUUGCAUAGU | hsa-let-7d |
| 1272 | UGAGGUAGGAGGUUGUAUAGU | hsa-let-7e |
| 1273 | UGAGGUAGUAGAUUGUAUAGUU | hsa-let-7f |
| 1274 | UGAGGUAGUAGUUUGUACAGU | hsa-let-7g |
| 1275 | UGAGGUAGUAGUUUGUGCUGU | hsa-let-7i |
| 1276 | UGGAAUGUAAAGAAGUAUGUA | hsa-miR-1 |
| 1277 | AACCCGUAGAUCCGAACUUGUG | hsa-miR-100 |
| 1278 | UACAGUACUGUGAUAACUGAAG | hsa-miR-101 |
| 1279 | AGCAGCAUUGUACAGGGCUAUGA | hsa-miR-103 |
| 1280 | UCAAAUGCUCAGACUCCUGU | hsa-miR-105 |
| 1281 | AAAAGUGCUUACAGUGCAGGUAGC | hsa-miR-106a |
| 1282 | UAAAGUGCUGACAGUGCAGAU | hsa-miR-106b |
| 1283 | AGCAGCAUUGUACAGGGCUAUCA | hsa-miR-107 |
| 1284 | AUAAGGAUUUUUAGGGGCAUU | hsa-miR-108 |
| 1285 | UACCCUGUAGAUCCGAAUUUGUG | hsa-miR-10a |
| 1286 | UACCCUGUAGAACCGAAUUUGU | hsa-miR-10b |
| 1287 | UGGAGUGUGACAAUGGUGUUUGU | hsa-miR-122a |
| 1288 | UUAAGGCACGCGGUGAAUGCCA | hsa-miR-124a |
| 1289 | UCCCUGAGACCCUUUAACCUGUG | hsa-miR-125a |
| 1290 | UCCCUGAGACCCUAACUUGUGA | hsa-miR-125b |
| 1291 | UCGUACCGUGAGUAAUAAUGC | hsa-miR-126 |
| 1292 | CAUUAUUACUUUUGGUACGCG | hsa-miR-126* |
| 1293 | UCGGAUCCGUCUGAGCUUGGCU | hsa-miR-127 |
| 1294 | UCACAGUGAACCGGUCUCUUUU | hsa-miR-128a |
| 1295 | UCACAGUGAACCGGUCUCUUUC | hsa-miR-128b |
| 1296 | CUUUUUGCGGUCUGGGCUUGC | hsa-miR-129 |
| 1297 | CAGUGCAAUGUUAAAAGGGCAU | hsa-miR-130a |
| 1298 | CAGUGCAAUGAUGAAAGGGCAU | hsa-miR-130b |
| 1299 | UAACAGUCUACAGCCAUGGUCG | hsa-miR-132 |
| 1300 | UUGGUCCCCUUCAACCAGCUGU | hsa-miR-133a |
| 1301 | UUGGUCCCCUUCAACCAGCUA | hsa-miR-133b |
| 1302 | UGUGACUGGUUGACCAGAGGG | hsa-miR-134 |
| 1303 | UAUGGCUUUUUAUUCCUAUGUGA | hsa-miR-135a |
| 1304 | UAUGGCUUUUCAUUCCUAUGUG | hsa-miR-135b |
| 1305 | ACUCCAUUUGUUUUGAUGAUGGA | hsa-miR-136 |
| 1306 | UAUUGCUUAAGAAUACGCGUAG | hsa-miR-137 |
| 1307 | AGCUGGUGUUGUGAAUC | hsa-miR-138 |
| 1308 | UCUACAGUGCACGUGUCU | hsa-miR-139 |
| 1309 | AGUGGUUUUACCCUAUGGUAG | hsa-miR-140 |
| 1310 | UAACACUGUCUGGUAAAGAUGG | hsa-miR-141 |
| 1311 | UGUAGUGUUUCCUACUUUAUGGA | hsa-miR-142-3p |
| 1312 | CAUAAAGUAGAAAGCACUAC | hsa-miR-142-5p |
| 1313 | UGAGAUGAAGCACUGUAGCUCA | hsa-miR-143 |
| 1314 | UACAGUAUAGAUGAUGUACUAG | hsa-miR-144 |
| 1315 | GUCCAGUUUUCCCAGGAAUCCCUU | hsa-miR-145 |
| 1316 | UGAGAACUGAAUUCCAUGGGUU | hsa-miR-146a |
| 1317 | UGAGAACUGAAUUCCAUAGGCU | hsa-miR-146b |
| 1318 | GUGUGUGGAAAUGCUUCUGC | hsa-miR-147 |
| 1319 | UCAGUGCACUACAGAACUUUGU | hsa-miR-148a |
| 1320 | UCAGUGCAUCACAGAACUUUGU | hsa-miR-148b |
| 1321 | UCUGGCUCCGUGUCUUCACUCC | hsa-miR-149 |
| 1322 | UCUCCCAACCCUUGUACCAGUG | hsa-miR-150 |
| 1323 | ACUAGACUGAAGCUCCUUGAGG | hsa-miR-151 |
| 1324 | UCAGUGCAUGACAGAACUUGGG | hsa-miR-152 |
| 1325 | UUGCAUAGUCACAAAAGUGA | hsa-miR-153 |
| 1326 | UAGGUUAUCCGUGUUGCCUUCG | hsa-miR-154 |
| 1327 | AAUCAUACACGGUUGACCUAUU | hsa-miR-154* |
| 1328 | UUAAUGCUAAUCGUGAUAGGGG | hsa-miR-155 |
| 1329 | UAGCAGCACAUAAUGGUUUGUG | hsa-miR-15a |
| 1330 | UAGCAGCACAUCAUGGUUUACA | hsa-miR-15b |
| 1331 | UAGCAGCACGUAAAUAUUGGCG | hsa-miR-16 |
| 1332 | ACUGCAGUGAAGGCACUUGU | hsa-miR-17-3p |
| 1333 | CAAAGUGCUUACAGUGCAGGUAGU | hsa-miR-17-5p |
| 1334 | AACAUUCAACGCUGUCGGUGAGU | hsa-miR-181a |
| 1335 | AACAUUCAUUGCUGUCGGUGGG | hsa-miR-181b |
| 1336 | AACAUUCAACCUGUCGGUGAGU | hsa-miR-181c |
| 1337 | AACAUUCAUUGUUGUCGGUGGGUU | hsa-miR-181d |
| 1338 | UUUGGCAAUGGUAGAACUCACA | hsa-miR-182 |
| 1339 | UGGUUCUAGACUUGCCAACUA | hsa-miR-182* |
| 1340 | UAUGGCACUGGUAGAAUUCACUG | hsa-miR-183 |
| 1341 | UGGACGGAGAACUGAUAAGGGU | hsa-miR-184 |
| 1342 | UGGAGAGAAAGGCAGUUC | hsa-miR-185 |
| 1343 | CAAAGAAUUCUCCUUUUGGGCUU | hsa-miR-186 |
| 1344 | UCGUGUCUUGUGUUGCAGCCG | hsa-miR-187 |
| 1345 | CAUCCCUUGCAUGGUGGAGGGU | hsa-miR-188 |
| 1346 | GUGCCUACUGAGCUGAUAUCAGU | hsa-miR-189 |
| 1347 | UAAGGUGCAUCUAGUGCAGAUA | hsa-miR-18a |
| 1348 | UAAGGUGCAUCUAGUGCAGUUA | hsa-miR-18b |
| 1349 | UGAUAUGUUUGAUAUAUUAGGU | hsa-miR-190 |
| 1350 | CAACGGAAUCCCAAAAGCAGCU | hsa-miR-191 |
| 1351 | GCUGCGCUUGGAUUUCGUCCCC | hsa-miR-191* |
| 1352 | CUGACCUAUGAAUUGACAGCC | hsa-miR-192 |
| 1353 | AACUGGCCUACAAAGUCCCAG | hsa-miR-193a |
| 1354 | AACUGGCCCUCAAAGUCCCGCUUU | hsa-miR-193b |
| 1355 | UGUAACAGCAACUCCAUGUGGA | hsa-miR-194 |
| 1356 | UAGCAGCACAGAAAUAUUGGC | hsa-miR-195 |
| 1357 | UAGGUAGUUUCAUGUUGUUGG | hsa-miR-196a |
| 1358 | UAGGUAGUUUCCUGUUGUUGG | hsa-miR-196b |
| 1359 | UUCACCACCUUCUCCACCCAGC | hsa-miR-197 |
| 1360 | GGUCCAGAGGGGAGAUAGG | hsa-miR-198 |
| 1361 | UACAGUAGUCUGCACAUUGGUU | hsa-miR-199a* |
| 1362 | CCCAGUGUUCAGACUACCUGUUC | hsa-miR-199a |
| 1363 | CCCAGUGUUUAGACUAUCUGUUC | hsa-miR-199b |
| 1364 | UGUGCAAAUCUAUGCAAAACUGA | hsa-miR-19a |
| 1365 | UGUGCAAAUCCAUGCAAAACUGA | hsa-miR-19b |
| 1366 | CAUCUUACCGGACAGUGCUGGA | hsa-miR-200a* |
| 1367 | UAACACUGUCUGGUAACGAUGU | hsa-miR-200a |
| 1368 | UAAUACUGCCUGGUAAUGAUGAC | hsa-miR-200b |
| 1369 | UAAUACUGCCGGGUAAUGAUGG | hsa-miR-200c |
| 1370 | AGAGGUAUAGGGCAUGGGAAAA | hsa-miR-202 |
| 1371 | UUUCCUAUGCAUAUACUUCUUU | hsa-miR-202* |
| 1372 | GUGAAAUGUUUAGGACCACUAG | hsa-miR-203 |
| 1373 | UUCCCUUUGUCAUCCUAUGCCU | hsa-miR-204 |
| 1374 | UCCUUCAUUCCACCGGAGUCUG | hsa-miR-205 |
| 1375 | UGGAAUGUAAGGAAGUGUGUGG | hsa-miR-206 |
| 1376 | AUAAGACGAGCAAAAAGCUUGU | hsa-miR-208 |
| 1377 | UAAAGUGCUUAUAGUGCAGGUAG | hsa-miR-20a |
| 1378 | CAAAGUGCUCAUAGUGCAGGUAG | hsa-miR-20b |
| 1379 | UAGCUUAUCAGACUGAUGUUGA | hsa-miR-21 |
| 1380 | CUGUGCGUGUGACAGCGGCUGA | hsa-miR-210 |
| 1381 | UUCCCUUUGUCAUCCUUCGCCU | hsa-miR-211 |
| 1382 | UAACAGUCUCCAGUCACGGCC | hsa-miR-212 |
| 1383 | ACCAUCGACCGUUGAUUGUACC | hsa-miR-213 |
| 1384 | ACAGCAGGCACAGACAGGCAG | hsa-miR-214 |
| 1385 | AUGACCUAUGAAUUGACAGAC | hsa-miR-215 |
| 1386 | UAAUCUCAGCUGGCAACUGUG | hsa-miR-216 |
| 1387 | UACUGCAUCAGGAACUGAUUGGAU | hsa-miR-217 |
| 1388 | UUGUGCUUGAUCUAACCAUGU | hsa-miR-218 |
| 1389 | UGAUUGUCCAAACGCAAUUCU | hsa-miR-219 |
| 1390 | AAGCUGCCAGUUGAAGAACUGU | hsa-miR-22 |
| 1391 | CCACACCGUAUCUGACACUUU | hsa-miR-220 |
| 1392 | AGCUACAUUGUCUGCUGGGUUUC | hsa-miR-221 |
| 1393 | AGCUACAUCUGGCUACUGGGUCUC | hsa-miR-222 |
| 1394 | UGUCAGUUUGUCAAAUACCCC | hsa-miR-223 |
| 1395 | CAAGUCACUAGUGGUUCCGUUUA | hsa-miR-224 |
| 1396 | AUCACAUUGCCAGGGAUUUCC | hsa-miR-23a |
| 1397 | AUCACAUUGCCAGGGAUUACC | hsa-miR-23b |
| 1398 | UGGCUCAGUUCAGCAGGAACAG | hsa-miR-24 |
| 1399 | CAUUGCACUUGUCUCGGUCUGA | hsa-miR-25 |
| 1400 | UUCAAGUAAUCCAGGAUAGGC | hsa-miR-26a |
| 1401 | UUCAAGUAAUUCAGGAUAGGUU | hsa-miR-26b |
| 1402 | UUCACAGUGGCUAAGUUCCGC | hsa-miR-27a |
| 1403 | UUCACAGUGGCUAAGUUCUGC | hsa-miR-27b |
| 1404 | AAGGAGCUCACAGUCUAUUGAG | hsa-miR-28 |
| 1405 | AGGGCCCCCCCUCAAUCCUGU | hsa-miR-296 |
| 1406 | UAUGUGGGAUGGUAAACCGCUU | hsa-miR-299-3p |
| 1407 | UGGUUUACCGUCCCACAUACAU | hsa-miR-299-5p |
| 1408 | UAGCACCAUCUGAAAUCGGUU | hsa-miR-29a |
| 1409 | UAGCACCAUUUGAAAUCAGUGUU | hsa-miR-29b |
| 1410 | UAGCACCAUUUGAAAUCGGU | hsa-miR-29c |
| 1411 | CAGUGCAAUAGUAUUGUCAAAGC | hsa-miR-301 |
| 1412 | UAAACGUGGAUGUACUUGCUUU | hsa-miR-302a* |
| 1413 | ACUUUAACAUGGAAGUGCUUUCU | hsa-miR-302b* |
| 1414 | UUUAACAUGGGGGUACCUGCUG | hsa-miR-302c* |
| 1415 | UAAGUGCUUCCAUGUUUUGGUGA | hsa-miR-302a |
| 1416 | UAAGUGCUUCCAUGUUUUAGUAG | hsa-miR-302b |
| 1417 | UAAGUGCUUCCAUGUUUCAGUGG | hsa-miR-302c |
| 1418 | UAAGUGCUUCCAUGUUUGAGUGU | hsa-miR-302d |
| 1419 | CUUUCAGUCGGAUGUUUGCAGC | hsa-miR-30a-3p |
| 1420 | UGUAAACAUCCUCGACUGGAAG | hsa-miR-30a-5p |
| 1421 | UGUAAACAUCCUACACUCAGCU | hsa-miR-30b |
| 1422 | UGUAAACAUCCUACACUCUCAGC | hsa-miR-30c |
| 1423 | UGUAAACAUCCCCGACUGGAAG | hsa-miR-30d |
| 1424 | CUUUCAGUCGGAUGUUUACAGC | hsa-miR-30e-3p |
| 1425 | UGUAAACAUCCUUGACUGGA | hsa-miR-30e-5p |
| 1426 | GGCAAGAUGCUGGCAUAGCUG | hsa-miR-31 |
| 1427 | UAUUGCACAUUACUAAGUUGC | hsa-miR-32 |
| 1428 | AAAAGCUGGGUUGAGAGGGCGAA | hsa-miR-320 |
| 1429 | GCACAUUACACGGUCGACCUCU | hsa-miR-323 |
| 1430 | CCACUGCCCCAGGUGCUGCUGG | hsa-miR-324-3p |
| 1431 | CGCAUCCCCUAGGGCAUUGGUGU | hsa-miR-324-5p |
| 1432 | CCUAGUAGGUGUCCAGUAAGUGU | hsa-miR-325 |
| 1433 | CCUCUGGGCCCUUCCUCCAG | hsa-miR-326 |
| 1434 | CUGGCCCUCUCUGCCCUUCCGU | hsa-miR-328 |
| 1435 | AACACACCUGGUUAACCUCUUU | hsa-miR-329 |
| 1436 | GUGCAUUGUAGUUGCAUUG | hsa-miR-33 |
| 1437 | GCAAAGCACACGGCCUGCAGAGA | hsa-miR-330 |
| 1438 | GCCCCUGGGCCUAUCCUAGAA | hsa-miR-331 |
| 1439 | UCAAGAGCAAUAACGAAAAAUGU | hsa-miR-335 |
| 1440 | UCCAGCUCCUAUAUGAUGCCUUU | hsa-miR-337 |
| 1441 | UCCAGCAUCAGUGAUUUUGUUGA | hsa-miR-338 |
| 1442 | UCCCUGUCCUCCAGGAGCUCA | hsa-miR-339 |
| 1443 | UCCGUCUCAGUUACUUUAUAGCC | hsa-miR-340 |
| 1444 | UCUCACACAGAAAUCGCACCCGUC | hsa-miR-342 |
| 1445 | UGCUGACUCCUAGUCCAGGGC | hsa-miR-345 |
| 1446 | UGUCUGCCCGCAUGCCUGCCUCU | hsa-miR-346 |
| 1447 | UGGCAGUGUCUUAGCUGGUUGUU | hsa-miR-34a |
| 1448 | UAGGCAGUGUCAUUAGCUGAUUG | hsa-miR-34b |
| 1449 | AGGCAGUGUAGUUAGCUGAUUGC | hsa-miR-34c |
| 1450 | UUAUCAGAAUCUCCAGGGGUAC | hsa-miR-361 |
| 1451 | AAUCCUUGGAACCUAGGUGUGAG | hsa-miR-362 |
| 1452 | AUUGCACGGUAUCCAUCUGUAA | hsa-miR-363 |
| 1453 | UAAUGCCCCUAAAAAUCCUUAU | hsa-miR-365 |
| 1454 | AAUUGCACUUUAGCAAUGGUGA | hsa-miR-367 |
| 1455 | ACAUAGAGGAAAUUCCACGUUU | hsa-miR-368 |
| 1456 | AAUAAUACAUGGUUGAUCUUU | hsa-miR-369-3p |
| 1457 | AGAUCGACCGUGUUAUAUUCGC | hsa-miR-369-5p |
| 1458 | GCCUGCUGGGGUGGAACCUGG | hsa-miR-370 |
| 1459 | GUGCCGCCAUCUUUUGAGUGU | hsa-miR-371 |
| 1460 | AAAGUGCUGCGACAUUUGAGCGU | hsa-miR-372 |
| 1461 | GAAGUGCUUCGAUUUUGGGGUGU | hsa-miR-373 |
| 1462 | ACUCAAAAUGGGGGCGCUUUCC | hsa-miR-373* |
| 1463 | UUAUAAUACAACCUGAUAAGUG | hsa-miR-374 |
| 1464 | UUUGUUCGUUCGGCUCGCGUGA | hsa-miR-375 |
| 1465 | AUCAUAGAGGAAAAUCCACGU | hsa-miR-376a |
| 1466 | AUCAUAGAGGAAAAUCCAUGUU | hsa-miR-376b |
| 1467 | AUCACACAAAGGCAACUUUUGU | hsa-miR-377 |
| 1468 | CUCCUGACUCCAGGUCCUGUGU | hsa-miR-378 |
| 1469 | UGGUAGACUAUGGAACGUA | hsa-miR-379 |
| 1470 | UAUGUAAUAUGGUCCACAUCUU | hsa-miR-380-3p |
| 1471 | UGGUUGACCAUAGAACAUGCGC | hsa-miR-380-5p |
| 1472 | UAUACAAGGGCAAGCUCUCUGU | hsa-miR-381 |
| 1473 | GAAGUUGUUCGUGGUGGAUUCG | hsa-miR-382 |
| 1474 | AGAUCAGAAGGUGAUUGUGGCU | hsa-miR-383 |
| 1475 | AUUCCUAGAAAUUGUUCAUA | hsa-miR-384 |
| 1476 | CGAAUGUUGCUCGGUGAACCCCU | hsa-miR-409-3p |
| 1477 | AGGUUACCCGAGCAACUUUGCA | hsa-miR-409-5p |
| 1478 | AAUAUAACACAGAUGGCCUGUU | hsa-miR-410 |
| 1479 | ACUUCACCUGGUCCACUAGCCGU | hsa-miR-412 |
| 1480 | CUGGACUUAGGGUCAGAAGGCC | hsa-miR-422a |
| 1481 | CUGGACUUGGAGUCAGAAGGCC | hsa-miR-422b |
| 1482 | AGCUCGGUCUGAGGCCCCUCAG | hsa-miR-423 |
| 1483 | CAGCAGCAAUUCAUGUUUUGAA | hsa-miR-424 |
| 1484 | AUCGGGAAUGUCGUGUCCGCC | hsa-miR-425 |
| 1485 | UAAUACUGUCUGGUAAAACCGU | hsa-miR-429 |
| 1486 | UGUCUUGCAGGCCGUCAUGCA | hsa-miR-431 |
| 1487 | UCUUGGAGUAGGUCAUUGGGUGG | hsa-miR-432 |
| 1488 | CUGGAUGGCUCCUCCAUGUCU | hsa-miR-432* |
| 1489 | AUCAUGAUGGGCUCCUCGGUGU | hsa-miR-433 |
| 1490 | UUGCAUAUGUAGGAUGUCCCAU | hsa-miR-448 |
| 1491 | UGGCAGUGUAUUGUUAGCUGGU | hsa-miR-449 |
| 1492 | UUUUUGCGAUGUGUUCCUAAUA | hsa-miR-450 |
| 1493 | AAACCGUUACCAUUACUGAGUUU | hsa-miR-451 |
| 1494 | UGUUUGCAGAGGAAACUGAGAC | hsa-miR-452 |
| 1495 | UCAGUCUCAUCUGCAAAGAAG | hsa-miR-452* |
| 1496 | GAGGUUGUCCGUGGUGAGUUCG | hsa-miR-453 |
| 1497 | GUCAUACACGGCUCUCCUCU | hsa-miR-485-3p |
| 1498 | AGAGGCUGGCCGUGAUGAAUUC | hsa-miR-485-5p |
| 1499 | CCCAGAUAAUGGCACUCUCAA | hsa-miR-488 |
| 1500 | AGUGACAUCACAUAUACGGCAGC | hsa-miR-489 |
| 1501 | CAACCUGGAGGACUCCAUGCUG | hsa-miR-490 |
| 1502 | AGUGGGGAACCCUUCCAUGAGGA | hsa-miR-491 |
| 1503 | AGGACCUGCGGGACAAGAUUCUU | hsa-miR-492 |
| 1504 | UUGUACAUGGUAGGCUUUCAUU | hsa-miR-493 |
| 1505 | UGAAACAUACACGGGAAACCUCUU | hsa-miR-494 |
| 1506 | AAACAAACAUGGUGCACUUCUUU | hsa-miR-495 |
| 1507 | AUUACAUGGCCAAUCUC | hsa-miR-496 |
| 1508 | CAGCAGCACACUGUGGUUUGU | hsa-miR-497 |
| 1509 | UUUCAAGCCAGGGGGCGUUUUUC | hsa-miR-498 |
| 1510 | UUAAGACUUGCAGUGAUGUUUAA | hsa-miR-499 |
| 1511 | AUGCACCUGGGCAAGGAUUCUG | hsa-miR-500 |
| 1512 | AAUCCUUUGUCCCUGGGUGAGA | hsa-miR-501 |
| 1513 | AUCCUUGCUAUCUGGGUGCUA | hsa-miR-502 |
| 1514 | UAGCAGCGGGAACAGUUCUGCAG | hsa-miR-503 |
| 1515 | AGACCCUGGUCUGCACUCUAU | hsa-miR-504 |
| 1516 | GUCAACACUUGCUGGUUUCCUC | hsa-miR-505 |
| 1517 | UAAGGCACCCUUCUGAGUAGA | hsa-miR-506 |
| 1518 | UUUUGCACCUUUUGGAGUGAA | hsa-miR-507 |
| 1519 | UGAUUGUAGCCUUUUGGAGUAGA | hsa-miR-508 |
| 1520 | UGAUUGGUACGUCUGUGGGUAGA | hsa-miR-509 |
| 1521 | UACUCAGGAGAGUGGCAAUCACA | hsa-miR-510 |
| 1522 | GUGUCUUUUGCUCUGCAGUCA | hsa-miR-511 |
| 1523 | AAGUGCUGUCAUAGCUGAGGUC | hsa-miR-512-3p |
| 1524 | CACUCAGCCUUGAGGGCACUUUC | hsa-miR-512-5p |
| 1525 | UUCACAGGGAGGUGUCAUUUAU | hsa-miR-513 |
| 1526 | AUUGACACUUCUGUGAGUAG | hsa-miR-514 |
| 1527 | GAGUGCCUUCUUUUGGAGCGU | hsa-miR-515-3p |
| 1528 | UUCUCCAAAAGAAAGCACUUUCUG | hsa-miR-515-5p |
| 1529 | UGCUUCCUUUCAGAGGGU | hsa-miR-516-3p |
| 1530 | AUCUGGAGGUAAGAAGCACUUU | hsa-miR-516-5p |
| 1531 | CCUCUAGAUGGAAGCACUGUCU | hsa-miR-517* |
| 1532 | AUCGUGCAUCCCUUUAGAGUGUU | hsa-miR-517a |
| 1533 | UCGUGCAUCCCUUUAGAGUGUU | hsa-miR-517b |
| 1534 | AUCGUGCAUCCUUUUAGAGUGU | hsa-miR-517c |
| 1535 | UCUGCAAAGGGAAGCCCUUU | hsa-miR-518a-2* |
| 1536 | UCUCUGGAGGGAAGCACUUUCUG | hsa-miR-518c* |
| 1537 | CUCUAGAGGGAAGCACUUUCUCU | hsa-miR-518f* |
| 1538 | AAAGCGCUUCCCUUUGCUGGA | hsa-miR-518a |
| 1539 | CAAAGCGCUCCCCUUUAGAGGU | hsa-miR-518b |
| 1540 | CAAAGCGCUUCUCUUUAGAGUG | hsa-miR-518c |
| 1541 | CAAAGCGCUUCCCUUUGGAGC | hsa-miR-518d |
| 1542 | AAAGCGCUUCCCUUCAGAGUGU | hsa-miR-518e |
| 1543 | AAAGCGCUUCUCUUUAGAGGA | hsa-miR-518f |
| 1544 | UUCUCCAAAAGGGAGCACUUUC | hsa-miR-519e* |
| 1545 | AAAGUGCAUCCUUUUAGAGUGUUAC | hsa-miR-519a |
| 1546 | AAAGUGCAUCCUUUUAGAGGUUU | hsa-miR-519b |
| 1547 | AAAGUGCAUCUUUUUAGAGGAU | hsa-miR-519c |
| 1548 | CAAAGUGCCUCCCUUUAGAGUGU | hsa-miR-519d |
| 1549 | AAAGUGCCUCCUUUUAGAGUGU | hsa-miR-519e |
| 1550 | CUCCAGAGGGAAGUACUUUCU | hsa-miR-520a* |
| 1551 | UCUACAAAGGGAAGCCCUUUCUG | hsa-miR-520d* |
| 1552 | AAAGUGCUUCCCUUUGGACUGU | hsa-miR-520a |
| 1553 | AAAGUGCUUCCUUUUAGAGGG | hsa-miR-520b |
| 1554 | AAAGUGCUUCCUUUUAGAGGGUU | hsa-miR-520c |
| 1555 | AAAGUGCUUCUCUUUGGUGGGUU | hsa-miR-520d |
| 1556 | AAAGUGCUUCCUUUUUGAGGG | hsa-miR-520e |
| 1557 | AAGUGCUUCCUUUUAGAGGGUU | hsa-miR-520f |
| 1558 | ACAAAGUGCUUCCCUUUAGAGUGU | hsa-miR-520g |
| 1559 | ACAAAGUGCUUCCCUUUAGAGU | hsa-miR-520h |
| 1560 | AACGCACUUCCCUUUAGAGUGU | hsa-miR-521 |
| 1561 | AAAAUGGUUCCCUUUAGAGUGUU | hsa-miR-522 |
| 1562 | AACGCGCUUCCCUAUAGAGGG | hsa-miR-523 |
| 1563 | GAAGGCGCUUCCCUUUGGAGU | hsa-miR-524 |
| 1564 | CUACAAAGGGAAGCACUUUCUC | hsa-miR-524* |
| 1565 | CUCCAGAGGGAUGCACUUUCU | hsa-miR-525 |
| 1566 | GAAGGCGCUUCCCUUUAGAGC | hsa-miR-525* |
| 1567 | AAAGUGCUUCCUUUUAGAGGC | hsa-miR-526b* |
| 1568 | CUCUAGAGGGAAGCACUUUCU | hsa-miR-526a |
| 1569 | CUCUUGAGGGAAGCACUUUCUGUU | hsa-miR-526b |
| 1570 | CUCUAGAGGGAAGCGCUUUCUGUU | hsa-miR-526c |
| 1571 | CUGCAAAGGGAAGCCCUUUCU | hsa-miR-527 |
| 1572 | UGGAAGACUAGUGAUUUUGUUG | hsa-miR-7 |
| 1573 | UCUUUGGUUAUCUAGCUGUAUGA | hsa-miR-9 |
| 1574 | UAAAGCUAGAUAACCGAAAGU | hsa-miR-9* |
| 1575 | UAUUGCACUUGUCCCGGCCUG | hsa-miR-92 |
| 1576 | AAAGUGCUGUUCGUGCAGGUAG | hsa-miR-93 |
| 1577 | UUCAACGGGUAUUUAUUGAGCA | hsa-miR-95 |
| 1578 | UUUGGCACUAGCACAUUUUUGC | hsa-miR-96 |
| 1579 | UGAGGUAGUAAGUUGUAUUGUU | hsa-miR-98 |
| 1580 | AACCCGUAGAUCCGAUCUUGUG | hsa-miR-99a |
| 1581 | CACCCGUAGAACCGACCUUGCG | hsa-miR-99b |

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties, provided that, if there is a conflict in definitions, the definitions provided herein shall control.

What is claimed is:

1. A probe set comprising at least five probes, each of the at least five probes having a target-complementary sequence independently selected from the group consisting of SEQ ID NOS: 1-1240.

2. The probe set of claim 1, wherein the probe set further includes at least one probe having a target-complementary sequence independently selected from the group consisting of SEQ ID NOS:1241-1250.

3. The probe set of claim 1, wherein each of said at least five probes in the probe set is characterized as having a Tm in the range from about 50° C. to about 60° C. when hybridized with its respective target miRNA.

4. The probe set of claim 1, wherein each of said at least five probes in the probe set is characterized as having a Tm in the range from about 55° C. to about 60° C. when hybridized with its respective target miRNA.

5. The probe set of claim 1, wherein each of said at least five probes is directed to a respective target miRNA, and wherein each of said at least five probes is not fully-complementary to its respective target miRNA.

6. The probe set of claim 1, wherein each of said at least five probes is directed to a respective target miRNA, and wherein each of at least four probes of said at least five probes is not fully-complementary to its respective target miRNA.

7. The probe set of claim 1, wherein the probe set comprises at least 20 probes.

8. The probe set of claim 1, wherein each of said at least 20 probes is directed to a respective target miRNA, and wherein each of at least 19 probes of said at least 20 probes is not fully-complementary to its respective target miRNA.

9. The probe set of claim 1, wherein each of said at least five probes comprises a linker sequence, the target-complementary sequence, and a Tm enhancement domain.

10. The probe set of claim 9, wherein the Tm enhancement domain of at least one of the at least five probes comprises a hairpin sequence.

11. The probe set of claim 1, wherein the target-complementary sequence of a first probe of said at least five probes differs from the target-complementary sequence of a second probe of said at least five probes by lacking at least one base relative to the target-complementary sequence of the second probe, wherein the first probe and second probe are directed to the same miRNA.

12. The probe set of claim 11, wherein the target-complementary sequence of the first probe differs from the target-complementary sequence of the second probe by lacking at least two bases relative to the target-complementary sequence of the second probe.

13. The probe set of claim 1, said probe set being directed to at least five different target miRNAs.

14. The probe set of claim 1, said probe set being directed to at least 20 different target miRNAs.

15. A array comprising:
an array support, and a probe set bound to said array support, the probe set comprising at least five probes, each of the at least five probes having a target-complementary sequence independently selected from the group consisting of SEQ ID NOS: 1-1240, wherein each of said at least five probes is present on said array support as a discrete feature.

16. The array of claim 15, wherein each of said at least five probes comprises a linker sequence, the target-complementary sequence, and a Tm enhancement domain, wherein the target-complementary sequence and the Tm enhancement domain for each probe is bound to the array support via the linker sequence of said probe.

17. The array of claim 15, wherein the Tm enhancement domain of at least one of the at least five probes comprises a hairpin sequence.

18. The array of claim 15, wherein each of said at least five probes is directed to a respective target miRNA, and wherein each of at least four probes of said at least five probes is not fully-complementary to its respective target miRNA.

19. The array of claim 15, wherein the probe set comprises at least 20 probes.

20. The array of claim 15, wherein the target-complementary sequence of a first probe of said at least five probes differs from the target-complementary sequence of a second probe of said at least five probes by lacking at least one base relative to the target-complementary sequence of the second probe, wherein the first probe and second probe are directed to the same miRNA.

21. A method of analyzing a sample for miRNAs, the method comprising:
contacting the sample with a array comprising a probe set, the probe set comprising at least five probes, each of the at least five probes having a target-complementary sequence independently selected from the group consisting of SEQ ID NOS: 1-1240, and
interrogating the array to obtain information about miRNAs in the sample.

22. The method of claim 21, wherein said contacting the sample with the array is performed under stringent assay conditions.

23. The method of claim 21, wherein contacting the sample with the array includes incubating the sample on the array at a temperature in the range from about 50° C. to about 60° C.

24. The method of claim 21, wherein each of said at least five probes in the probe set is characterized as having a Tm in the range from about 50° C. to about 60° C. when hybridized with its respective target miRNA.

25. The method of claim 21, wherein each of said at least five probes is directed to a respective target miRNA, and wherein each of at least four probes of said at least five probes is not fully-complementary to its respective target miRNA.

26. The method of claim 21, wherein the probe set comprises at least 20 probes.

27. The method of claim 26, wherein each of said at least 20 probes is directed to a respective target miRNA, and wherein each of at least 19 probes of said at least 20 probes is not fully-complementary to its respective target miRNA.

28. The method of claim 21, wherein the target-complementary sequence of a first probe of said at least five probes differs from the target-complementary sequence of a second probe of said at least five probes by lacking at least one base relative to the target-complementary sequence of the second probe, wherein the first probe and second probe are directed to the same miRNA.


SEQUENCE LISTING (1581 sequences)

SEQ ID NO: 1 - DNA, 18 bp, Drosophila melanogaster
gcactgatttcgaatggt

SEQ ID NO: 2 - DNA, 15 bp, Drosophila melanogaster
gctcctcaaagctgg

SEQ ID NO: 3 - DNA, 17 bp, Drosophila melanogaster
tgagacacactttgccc

SEQ ID NO: 4 - DNA, 22 bp, Drosophila melanogaster
ctcacagtataatcctgtgatt

SEQ ID NO: 5 - DNA, 18 bp, Drosophila melanogaster
tcagctatgccgacatct

SEQ ID NO: 6 - DNA, 19 bp, Drosophila melanogaster
aaaaagaacagccactgtg

SEQ ID NO: 7 - DNA, 19 bp, Homo sapiens
aactatacaacctactacc

SEQ ID NO: 8 - DNA, 20 bp, Homo sapiens
aactatacaacctactacct

SEQ ID NO: 9 - DNA, 17 bp, Homo sapiens
aaccacacaacctacta

SEQ ID NO: 10 - DNA, 18 bp, Homo sapiens
aaccacacaacctactac

SEQ ID NO: 11 - DNA, 18 bp, Homo sapiens
aaccatacaacctactac

SEQ ID NO: 12 - DNA, 19 bp, Homo sapiens
aaccatacaacctactacc

SEQ ID NO: 13 - DNA, 20 bp, Homo sapiens
aaccatacaacctactacct

SEQ ID NO: 14 - DNA, 19 bp, Homo sapiens
actatgcaacctactacct

SEQ ID NO: 15 - DNA, 20 bp, Homo sapiens
actatacaacctcctacctc

SEQ ID NO: 16 - DNA, 21 bp, Homo sapiens
actatacaacctcctacctca

SEQ ID NO: 17 - DNA, 20 bp, Homo sapiens
aactatacaatctactacct

SEQ ID NO: 18 - DNA, 21 bp, Homo sapiens
aactatacaatctactacctc

SEQ ID NO: 19 - DNA, 22 bp, Homo sapiens
aactatacaatctactacctca

SEQ ID NO: 20 - DNA, 19 bp, Homo sapiens
actgtacaaactactacct

SEQ ID NO: 21 - DNA, 20 bp, Homo sapiens
actgtacaaactactacctc

SEQ ID NO: 22 - DNA, 21 bp, Homo sapiens
actgtacaaactactacctca

SEQ ID NO: 23 - DNA, 18 bp, Homo sapiens
acagcacaaactactacc

SEQ ID NO: 24 - DNA, 19 bp, Homo sapiens
acagcacaaactactacct

SEQ ID NO: 25 - DNA, 20 bp, Homo sapiens
acagcacaaactactacctc

SEQ ID NO: 26 - DNA, 20 bp, Homo sapiens
tacatacttctttacattcc

SEQ ID NO: 27 - DNA, 21 bp, Homo sapiens
tacatacttctttacattcca

SEQ ID NO: 28 - DNA, 17 bp, Homo sapiens
cacaagttcggatctac

SEQ ID NO: 29 - DNA, 18 bp, Homo sapiens
cacaagttcggatctacg

SEQ ID NO: 30 - DNA, 22 bp, Homo sapiens
cttcagttatcacagtactgta

SEQ ID NO: 31 - DNA, 17 bp, Homo sapiens
tcatagccctgtacaat

SEQ ID NO: 32 - DNA, 18 bp, Homo sapiens
tcatagccctgtacaatg

SEQ ID NO: 33 - DNA, 15 bp, Homo sapiens
acaggagtctgagca

SEQ ID NO: 34 - DNA, 16 bp, Homo sapiens
acaggagtctgagcat

SEQ ID NO: 35 - DNA, 17 bp, Homo sapiens
acaggagtctgagcatt

SEQ ID NO: 36 - DNA, 18 bp, Homo sapiens
acaggagtctgagcattt

SEQ ID NO: 37 - DNA, 19 bp, Homo sapiens
acaggagtctgagcatttg

SEQ ID NO: 38 - DNA, 17 bp, Homo sapiens
gctacctgcactgtaag

SEQ ID NO: 39 - DNA, 16 bp, Homo sapiens
atctgcactgtcagca

SEQ ID NO: 40 - DNA, 19 bp, Homo sapiens
tgatagccctgtacaatgc

SEQ ID NO: 41 - DNA, 17 bp, Homo sapiens
aatgcccctaaaaatcc

SEQ ID NO: 42 - DNA, 18 bp, Homo sapiens
aatgcccctaaaaatcct

SEQ ID NO: 43 - DNA, 19 bp, Homo sapiens
aatgcccctaaaaatcctt

SEQ ID NO: 44 - DNA, 20 bp, Homo sapiens
aatgcccctaaaaatcctta

SEQ ID NO: 45 - DNA, 21 bp, Homo sapiens
aatgcccctaaaaatccttat

SEQ ID NO: 46 - DNA, 19 bp, Homo sapiens
cacaaattcggatctacag

SEQ ID NO: 47 - DNA, 20 bp, Homo sapiens
cacaaattcggatctacagg

SEQ ID NO: 48 - DNA, 21 bp, Homo sapiens
cacaaattcggatctacaggg

SEQ ID NO: 49 - DNA, 19 bp, Homo sapiens
acaaattcggttctacagg

SEQ ID NO: 50 - DNA, 16 bp, Homo sapiens
acaaacaccattgtca

SEQ ID NO: 51 - DNA, 17 bp, Homo sapiens
acaaacaccattgtcac

SEQ ID NO: 52 - DNA, 18 bp, Homo sapiens
acaaacaccattgtcaca

SEQ ID NO: 53 - DNA, 19 bp, Homo sapiens
acaaacaccattgtcacac

SEQ ID NO: 54 - DNA, 13 bp, Homo sapiens
tggcattcaccgc

SEQ ID NO: 55 - DNA, 14 bp, Homo sapiens
tggcattcaccgcg

SEQ ID NO: 56 - DNA, 15 bp, Homo sapiens
tggcattcaccgcgt

SEQ ID NO: 57 - DNA, 18 bp, Homo sapiens
cacaggttaaagggtctc

SEQ ID NO: 58 - DNA, 19 bp, Homo sapiens
cacaggttaaagggtctca

SEQ ID NO: 59 - DNA, 20 bp, Homo sapiens
cacaggttaaagggtctcag

SEQ ID NO: 60 - DNA, 17 bp, Homo sapiens
tcacaagttagggtctc

SEQ ID NO: 61 - DNA, 18 bp, Homo sapiens
tcacaagttagggtctca

SEQ ID NO: 62 - DNA, 19 bp, Homo sapiens
gcattattactcacggtac

SEQ ID NO: 63 - DNA, 20 bp, Homo sapiens
gcattattactcacggtacg

SEQ ID NO: 64 - DNA, 21 bp, Homo sapiens
cgcgtaccaaaagtaataatg

SEQ ID NO: 65 - DNA, 16 bp, Homo sapiens
agccaagctcagacgg

SEQ ID NO: 66 - DNA, 15 bp, Homo sapiens
aaaagagaccggttc

SEQ ID NO: 67 - DNA, 16 bp, Homo sapiens
aaaagagaccggttca

SEQ ID NO: 68 - DNA, 17 bp, Homo sapiens
aaaagagaccggttcac

SEQ ID NO: 69 - DNA, 18 bp, Homo sapiens
aaaagagaccggttcact

SEQ ID NO: 70 - DNA, 17 bp, Homo sapiens
gaaagagaccggttcac

SEQ ID NO: 71 - DNA, 18 bp, Homo sapiens
gaaagagaccggttcact

SEQ ID NO: 72 - DNA, 19 bp, Homo sapiens
gaaagagaccggttcactg

SEQ ID NO: 73 - DNA, 13 bp, Homo sapiens
gcaagcccagacc

SEQ ID NO: 74 - DNA, 14 bp, Homo sapiens
gcaagcccagaccg

SEQ ID NO: 75 - DNA, 18 bp, Homo sapiens
atgcccttttaacattgc

SEQ ID NO: 76 - DNA, 19 bp, Homo sapiens
atgcccttttaacattgca

SEQ ID NO: 77 - DNA, 17 bp, Homo sapiens
atgccctttcatcattg

SEQ ID NO: 78 - DNA, 15 bp, Homo sapiens
cgaccatggctgtag

SEQ ID NO: 79 - DNA, 16 bp, Homo sapiens
cgaccatggctgtaga

SEQ ID NO: 80 - DNA, 16 bp, Homo sapiens
acagctggttgaaggg

SEQ ID NO: 81 - DNA, 17 bp, Homo sapiens
acagctggttgaagggg

SEQ ID NO: 82 - DNA, 18 bp, Homo sapiens
acagctggttgaagggga

SEQ ID NO: 83 - DNA, 17 bp, Homo sapiens
tagctggttgaagggga

SEQ ID NO: 84 - DNA, 18 bp, Homo sapiens
tagctggttgaaggggac

SEQ ID NO: 85 - DNA, 19 bp, Homo sapiens
tagctggttgaaggggacc

SEQ ID NO: 86 - DNA, 14 bp, Homo sapiens
ccctctggtcaacc

SEQ ID NO: 87 - DNA, 15 bp, Homo sapiens
ccctctggtcaacca

SEQ ID NO: 88 - DNA, 21 bp, Homo sapiens
tcacataggaataaaaagcca

SEQ ID NO: 89 - DNA, 22 bp, Homo sapiens
tcacataggaataaaaagccat

SEQ ID NO: 90 - DNA, 23 bp, Homo sapiens
tcacataggaataaaaagccata

SEQ ID NO: 91 - DNA, 18 bp, Homo sapiens
cacataggaatgaaaagc

SEQ ID NO: 92 - DNA, 19 bp, Homo sapiens
cacataggaatgaaaagcc

SEQ ID NO: 93 - DNA, 21 bp, Homo sapiens
tccatcatcaaaacaaatgga

SEQ ID NO: 94 - DNA, 17 bp, Homo sapiens
ctacgcgtattcttaag

SEQ ID NO: 95 - DNA, 18 bp, Homo sapiens
ctacgcgtattcttaagc

SEQ ID NO: 96 - DNA, 19 bp, Homo sapiens
ctacgcgtattcttaagca

SEQ ID NO: 97 - DNA, 20 bp, Homo sapiens
ctacgcgtattcttaagcaa

SEQ ID NO: 98 - DNA, 21 bp, Homo sapiens
ctacgcgtattcttaagcaat

SEQ ID NO: 99 - DNA, 16 bp, Homo sapiens
gattcacaacaccagc

SEQ ID NO: 100 - DNA, 17 bp, Homo sapiens
gattcacaacaccagct

SEQ ID NO: 101 - DNA, 17 bp, Homo sapiens
agacacgtgcactgtag

SEQ ID NO: 102 - DNA, 18 bp, Homo sapiens
agacacgtgcactgtaga

SEQ ID NO: 103 - DNA, 19 bp, Homo sapiens
ctaccatagggtaaaacca

SEQ ID NO: 104 - DNA, 20 bp, Homo sapiens
ctaccatagggtaaaaccac

SEQ ID NO: 105 - DNA, 16 bp, Homo sapiens
ccatctttaccagaca

SEQ ID NO: 106 - DNA, 17 bp, Homo sapiens
ccatctttaccagacag

SEQ ID NO: 107 - DNA, 18 bp, Homo sapiens
ccatctttaccagacagt

SEQ ID NO: 108 - DNA, 19 bp, Homo sapiens
ccatctttaccagacagtg

SEQ ID NO: 109 - DNA, 22 bp, Homo sapiens
tccataaagtaggaaacactac

SEQ ID NO: 110 - DNA, 19 bp, Homo sapiens
gtagtgctttctactttat

SEQ ID NO: 111 - DNA, 20 bp, Homo sapiens
gtagtgctttctactttatg

SEQ ID NO: 112 - DNA, 19 bp, Homo sapiens
tgagctacagtgcttcatc

SEQ ID NO: 113 - DNA, 22 bp, Homo sapiens
ctagtacatcatctatactgta

SEQ ID NO: 114 - DNA, 18 bp, Homo sapiens
aagggattcctgggaaaa

SEQ ID NO: 115 - DNA, 19 bp, Homo sapiens
aagggattcctgggaaaac

SEQ ID NO: 116 - DNA, 20 bp, Homo sapiens
aagggattcctgggaaaact

SEQ ID NO: 117 - DNA, 16 bp, Homo sapiens
aacccatggaattcag

SEQ ID NO: 118 - DNA, 17 bp, Homo sapiens
aacccatggaattcagt

SEQ ID NO: 119 - DNA, 18 bp, Homo sapiens
aacccatggaattcagtt

SEQ ID NO: 120 - DNA, 19 bp, Homo sapiens
aacccatggaattcagttc

SEQ ID NO: 121 - DNA, 20 bp, Homo sapiens
aacccatggaattcagttct

SEQ ID NO: 122 - DNA, 17 bp, Homo sapiens
agcctatggaattcagt

SEQ ID NO: 123 - DNA, 18 bp, Homo sapiens
agcctatggaattcagtt

SEQ ID NO: 124 - DNA, 19 bp, Homo sapiens
agcctatggaattcagttc

SEQ ID NO: 125 - DNA, 20 bp, Homo sapiens
agcctatggaattcagttct

SEQ ID NO: 126 - DNA, 16 bp, Homo sapiens
gcagaagcatttccac

SEQ ID NO: 127 - DNA, 17 bp, Homo sapiens
gcagaagcatttccaca

SEQ ID NO: 128 - DNA, 19 bp, Homo sapiens
acaaagttctgtagtgcac

SEQ ID NO: 129 - DNA, 20 bp, Homo sapiens
acaaagttctgtagtgcact

SEQ ID NO: 130 - DNA, 19 bp, Homo sapiens
acaaagttctgtgatgcac

SEQ ID NO: 131 - DNA, 20 bp, Homo sapiens
acaaagttctgtgatgcact

SEQ ID NO: 132 - DNA, 14 bp, Homo sapiens
ggagtgaagacacg

SEQ ID NO: 133 - DNA, 15 bp, Homo sapiens
ggagtgaagacacgg

SEQ ID NO: 134 - DNA, 16 bp, Homo sapiens
ggagtgaagacacgga

SEQ ID NO: 135 - DNA, 14 bp, Homo sapiens
cactggtacaaggg

SEQ ID NO: 136 - DNA, 15 bp, Homo sapiens
cactggtacaagggt

SEQ ID NO: 137 - DNA, 16 bp, Homo sapiens
cactggtacaagggtt

SEQ ID NO: 138 - DNA, 17 bp, Homo sapiens
cactggtacaagggttg

SEQ ID NO: 139 - DNA, 16 bp, Homo sapiens
cctcaaggagcttcag

SEQ ID NO: 140 - DNA, 17 bp, Homo sapiens
cctcaaggagcttcagt

SEQ ID NO: 141 - DNA, 17 bp, Homo sapiens
cccaagttctgtcatgc

SEQ ID NO: 142 - DNA, 18 bp, Homo sapiens
tcacttttgtgactatgc

SEQ ID NO: 143 - DNA, 19 bp, Homo sapiens
tcacttttgtgactatgca

SEQ ID NO: 144 - DNA, 20 bp, Homo sapiens
tcacttttgtgactatgcaa

SEQ ID NO: 145 - DNA, 13 bp, Homo sapiens
cgaaggcaacacg

SEQ ID NO: 146 - DNA, 14 bp, Homo sapiens
cgaaggcaacacgg

SEQ ID NO: 147 - DNA, 15 bp, Homo sapiens
cgaaggcaacacgga

SEQ ID NO: 148 - DNA, 16 bp, Homo sapiens
aataggtcaaccgtgt

SEQ ID NO: 149 - DNA, 17 bp, Homo sapiens
aataggtcaaccgtgta

SEQ ID NO: 150 - DNA, 18 bp, Homo sapiens
aataggtcaaccgtgtat

SEQ ID NO: 151 - DNA, 19 bp, Homo sapiens
aataggtcaaccgtgtatg

SEQ ID NO: 152 - DNA, 20 bp, Homo sapiens
aataggtcaaccgtgtatga

SEQ ID NO: 153 - DNA, 21 bp, Homo sapiens
aataggtcaaccgtgtatgat

SEQ ID NO: 154 - DNA, 16 bp, Homo sapiens
cccctatcacgattag

SEQ ID NO: 155 - DNA, 17 bp, Homo sapiens
cccctatcacgattagc

SEQ ID NO: 156 - DNA, 18 bp, Homo sapiens
cccctatcacgattagca

SEQ ID NO: 157 - DNA, 19 bp, Homo sapiens
cccctatcacgattagcat

SEQ ID NO: 158 - DNA, 19 bp, Homo sapiens
cacaaaccattatgtgctg

SEQ ID NO: 159 - DNA, 20 bp, Homo sapiens
cacaaaccattatgtgctgc

SEQ ID NO: 160 - DNA, 21 bp, Homo sapiens
cacaaaccattatgtgctgct

SEQ ID NO: 161 - DNA, 17 bp, Homo sapiens
tgtaaaccatgatgtgc

SEQ ID NO: 162 - DNA, 18 bp, Homo sapiens
tgtaaaccatgatgtgct

SEQ ID NO: 163 - DNA, 19 bp, Homo sapiens
tgtaaaccatgatgtgctg

SEQ ID NO: 164 - DNA, 17 bp, Homo sapiens
cgccaatatttacgtgc

SEQ ID NO: 165 - DNA, 18 bp, Homo sapiens
cgccaatatttacgtgct

SEQ ID NO: 166 - DNA, 17 bp, Homo sapiens
acaagtgccttcactgc

SEQ ID NO: 167 - DNA, 18 bp, Homo sapiens
actacctgcactgtaagc

SEQ ID NO: 168 - DNA, 19 bp, Homo sapiens
actacctgcactgtaagca

SEQ ID NO: 169 - DNA, 14 bp, Homo sapiens
actcaccgacagcg

SEQ ID NO: 170 - DNA, 15 bp, Homo sapiens
actcaccgacagcgt

SEQ ID NO: 171 - DNA, 16 bp, Homo sapiens
actcaccgacagcgtt

SEQ ID NO: 172 - DNA, 14 bp, Homo sapiens
cccaccgacagcaa

SEQ ID NO: 173 - DNA, 15 bp, Homo sapiens
cccaccgacagcaat

SEQ ID NO: 174 - DNA, 17 bp, Homo sapiens
actcaccgacaggttga

SEQ ID NO: 175 - DNA, 14 bp, Homo sapiens
aacccaccgacaac

SEQ ID NO: 176 - DNA, 15 bp, Homo sapiens
aacccaccgacaaca

SEQ ID NO: 177 - DNA, 16 bp, Homo sapiens
aacccaccgacaacaa

SEQ ID NO: 178 - DNA, 17 bp, Homo sapiens
aacccaccgacaacaat

SEQ ID NO: 179 - DNA, 17 bp, Homo sapiens
tgtgagttctaccattg

SEQ ID NO: 180 - DNA, 18 bp, Homo sapiens
tgtgagttctaccattgc

SEQ ID NO: 181 - DNA, 21 bp, Homo sapiens
tagttggcaagtctagaacca

SEQ ID NO: 182 - DNA, 17 bp, Homo sapiens
tagttggcaagtctaga

SEQ ID NO: 183 - DNA, 18 bp, Homo sapiens
tagttggcaagtctagaa

SEQ ID NO: 184 - DNA, 19 bp, Homo sapiens
tagttggcaagtctagaac

SEQ ID NO: 185 - DNA, 20 bp, Homo sapiens
tagttggcaagtctagaacc

SEQ ID NO: 186 - DNA, 17 bp, Homo sapiens
cagtgaattctaccagt

SEQ ID NO: 187 - DNA, 18 bp, Homo sapiens
cagtgaattctaccagtg

SEQ ID NO: 188 - DNA, 19 bp, Homo sapiens
cagtgaattctaccagtgc

SEQ ID NO: 189 - DNA, 18 bp, Homo sapiens
acccttatcagttctccg

SEQ ID NO: 190 - DNA, 19 bp, Homo sapiens
acccttatcagttctccgt

SEQ ID NO: 191 - DNA, 20 bp, Homo sapiens
acccttatcagttctccgtc

SEQ ID NO: 192 - DNA, 15 bp, Homo sapiens
gaactgcctttctct

SEQ ID NO: 193 - DNA, 19 bp, Homo sapiens
aagcccaaaaggagaattc

SEQ ID NO: 194 - DNA, 20 bp, Homo sapiens
aagcccaaaaggagaattct

SEQ ID NO: 195 - DNA, 21 bp, Homo sapiens
aagcccaaaaggagaattctt

SEQ ID NO: 196 - DNA, 12 bp, Homo sapiens
cggctgcaacac

SEQ ID NO: 197 - DNA, 13 bp, Homo sapiens
cggctgcaacaca

SEQ ID NO: 198 - DNA, 14 bp, Homo sapiens
cggctgcaacacaa

SEQ ID NO: 199 - DNA, 15 bp, Homo sapiens
cggctgcaacacaag

SEQ ID NO: 200 - DNA, 14 bp, Homo sapiens
accctccaccatgc

SEQ ID NO: 201 - DNA, 15 bp, Homo sapiens
accctccaccatgca

SEQ ID NO: 202 - DNA, 20 bp, Homo sapiens
actgatatcagctcagtagg

SEQ ID NO: 203 - DNA, 17 bp, Homo sapiens
tatctgcactagatgca

SEQ ID NO: 204 - DNA, 18 bp, Homo sapiens
tatctgcactagatgcac

SEQ ID NO: 205 - DNA, 19 bp, Homo sapiens
tatctgcactagatgcacc

SEQ ID NO: 206 - DNA, 20 bp, Homo sapiens
tatctgcactagatgcacct

SEQ ID NO: 207 - DNA, 17 bp, Homo sapiens
taactgcactagatgca

SEQ ID NO: 208 - DNA, 18 bp, Homo sapiens
taactgcactagatgcac

SEQ ID NO: 209 - DNA, 19 bp, Homo sapiens
taactgcactagatgcacc

SEQ ID NO: 210 - DNA, 22 bp, Homo sapiens
acctaatatatcaaacatatca

SEQ ID NO: 211 - DNA, 14 bp, Homo sapiens
agctgcttttggga

SEQ ID NO: 212 - DNA, 15 bp, Homo sapiens
agctgcttttgggat

SEQ ID NO: 213 - DNA, 16 bp, Homo sapiens
agctgcttttgggatt

SEQ ID NO: 214 - DNA, 13 bp, Homo sapiens
ggggacgaaatcc

SEQ ID NO: 215 - DNA, 14 bp, Homo sapiens
ggggacgaaatcca

SEQ ID NO: 216 - DNA, 15 bp, Homo sapiens
ggggacgaaatccaa

SEQ ID NO: 217 - DNA, 16 bp, Homo sapiens
ggggacgaaatccaag

SEQ ID NO: 218 - DNA, 16 bp, Homo sapiens
ggctgtcaattcatag

SEQ ID NO: 219 - DNA, 17 bp, Homo sapiens
ggctgtcaattcatagg

SEQ ID NO: 220 - DNA, 18 bp, Homo sapiens
ggctgtcaattcataggt

SEQ ID NO: 221 - DNA, 19 bp, Homo sapiens
ggctgtcaattcataggtc

SEQ ID NO: 222 - DNA, 15 bp, Homo sapiens
ctgggactttgtagg

SEQ ID NO: 223 - DNA, 16 bp, Homo sapiens
ctgggactttgtaggc

SEQ ID NO: 224 - DNA, 15 bp, Homo sapiens
aaagcgggactttga

SEQ ID NO: 225 - DNA, 16 bp, Homo sapiens
aaagcgggactttgag

SEQ ID NO: 226 - DNA, 17 bp, Homo sapiens
aaagcgggactttgagg

SEQ ID NO: 227 - DNA, 15 bp, Homo sapiens
tccacatggagttgc

SEQ ID NO: 228 - DNA, 16 bp, Homo sapiens
tccacatggagttgct

SEQ ID NO: 229 - DNA, 18 bp, Homo sapiens
gccaatatttctgtgctg

SEQ ID NO: 230 - DNA, 16 bp, Homo sapiens
ccaacaacatgaaact

SEQ ID NO: 231 - DNA, 17 bp, Homo sapiens
ccaacaacatgaaacta

SEQ ID NO: 232 - DNA, 18 bp, Homo sapiens
ccaacaacatgaaactac

SEQ ID NO: 233 - DNA, 19 bp, Homo sapiens
ccaacaacatgaaactacc

SEQ ID NO: 234 - DNA, 15 bp, Homo sapiens
ccaacaacaggaaac

SEQ ID NO: 235 - DNA, 16 bp, Homo sapiens
ccaacaacaggaaact

SEQ ID NO: 236 - DNA, 17 bp, Homo sapiens
ccaacaacaggaaacta

SEQ ID NO: 237 - DNA, 18 bp, Homo sapiens
ccaacaacaggaaactac

SEQ ID NO: 238 - DNA, 13 bp, Homo sapiens
gctgggtggagaa

SEQ ID NO: 239 - DNA, 14 bp, Homo sapiens
gctgggtggagaag

SEQ ID NO: 240 - DNA, 15 bp, Homo sapiens
gctgggtggagaagg

SEQ ID NO: 241 - DNA, 15 bp, Homo sapiens
cctatctcccctctg

SEQ ID NO: 242 - DNA, 16 bp, Homo sapiens
cctatctcccctctgg

SEQ ID NO: 243 - DNA, 17 bp, Homo sapiens
cctatctcccctctgga

SEQ ID NO: 244 - DNA, 19 bp, Homo sapiens
gaacaggtagtctgaacac

SEQ ID NO: 245 - DNA, 18 bp, Homo sapiens
aaccaatgtgcagactac

SEQ ID NO: 246 - DNA, 19 bp, Homo sapiens
aaccaatgtgcagactact

SEQ ID NO: 247 - DNA, 21 bp, Homo sapiens
gaacagatagtctaaacactg

SEQ ID NO: 248 - DNA, 20 bp, Homo sapiens
tcagttttgcatagatttgc

SEQ ID NO: 249 - DNA, 21 bp, Homo sapiens
tcagttttgcatagatttgca

SEQ ID NO: 250 - DNA, 19 bp, Homo sapiens
tcagttttgcatggatttg

SEQ ID NO: 251 - DNA, 17 bp, Homo sapiens
acatcgttaccagacag

SEQ ID NO: 252 - DNA, 18 bp, Homo sapiens
acatcgttaccagacagt

SEQ ID NO: 253 - DNA, 14 bp, Homo sapiens
tccagcactgtccg

SEQ ID NO: 254 - DNA, 15 bp, Homo sapiens
tccagcactgtccgg

SEQ ID NO: 255 - DNA, 16 bp, Homo sapiens
gtcatcattaccaggc

SEQ ID NO: 256 - DNA, 17 bp, Homo sapiens
gtcatcattaccaggca

SEQ ID NO: 257 - DNA, 18 bp, Homo sapiens
gtcatcattaccaggcag

SEQ ID NO: 258 - DNA, 14 bp, Homo sapiens
ccatcattacccgg

SEQ ID NO: 259 - DNA, 15 bp, Homo sapiens
ccatcattacccggc

SEQ ID NO: 260 - DNA, 16 bp, Homo sapiens
ccatcattacccggca

SEQ ID NO: 261 - DNA, 14 bp, Homo sapiens
ttttcccatgccct

SEQ ID NO: 262 - DNA, 15 bp, Homo sapiens
ttttcccatgcccta

SEQ ID NO: 263 - DNA, 16 bp, Homo sapiens
ttttcccatgccctat

SEQ ID NO: 264 - DNA, 17 bp, Homo sapiens
ttttcccatgccctata

SEQ ID NO: 265 - DNA, 18 bp, Homo sapiens
ttttcccatgccctatac

SEQ ID NO: 266 - DNA, 19 bp, Homo sapiens
ttttcccatgccctatacc

SEQ ID NO: 267 - DNA, 20 bp, Homo sapiens
aaagaagtatatgcatagga

SEQ ID NO: 268 - DNA, 21 bp, Homo sapiens
aaagaagtatatgcataggaa

SEQ ID NO: 269 - DNA, 22 bp, Homo sapiens
aaagaagtatatgcataggaaa

SEQ ID NO: 270 - DNA, 16 bp, Homo sapiens
ctagtggtcctaaaca

SEQ ID NO: 271 - DNA, 17 bp, Homo sapiens
ctagtggtcctaaacat

SEQ ID NO: 272 - DNA, 18 bp, Homo sapiens
ctagtggtcctaaacatt

SEQ ID NO: 273 - DNA, 19 bp, Homo sapiens
ctagtggtcctaaacattt

SEQ ID NO: 274 - DNA, 20 bp, Homo sapiens
ctagtggtcctaaacatttc

SEQ ID NO: 275 - DNA, 21 bp, Homo sapiens
ctagtggtcctaaacatttca

SEQ ID NO: 276 - DNA, 19 bp, Homo sapiens
aggcataggatgacaaagg

SEQ ID NO: 277 - DNA, 13 bp, Homo sapiens
cagactccggtgg

SEQ ID NO: 278 - DNA, 14 bp, Homo sapiens
cagactccggtgga

SEQ ID NO: 279 - DNA, 15 bp, Homo sapiens
cagactccggtggaa

SEQ ID NO: 280 - DNA, 16 bp, Homo sapiens
cagactccggtggaat

SEQ ID NO: 281 - DNA, 14 bp, Homo sapiens
ccacacacttcctt

SEQ ID NO: 282 - DNA, 15 bp, Homo sapiens
ccacacacttcctta

SEQ ID NO: 283 - DNA, 16 bp, Homo sapiens
ccacacacttccttac

SEQ ID NO: 284 - DNA, 17 bp, Homo sapiens
ccacacacttccttaca

SEQ ID NO: 285 - DNA, 18 bp, Homo sapiens
ccacacacttccttacat

SEQ ID NO: 286 - DNA, 19 bp, Homo sapiens
ccacacacttccttacatt

SEQ ID NO: 287 - DNA, 16 bp, Homo sapiens
acaagctttttgctcg

SEQ ID NO: 288 - DNA, 18 bp, Homo sapiens
acaagctttttgctcgtc

SEQ ID NO: 289 - DNA, 19 bp, Homo sapiens
acaagctttttgctcgtct

SEQ ID NO: 290 - DNA, 17 bp, Homo sapiens
ctacctgcactataagc

SEQ ID NO: 291 - DNA, 18 bp, Homo sapiens
ctacctgcactataagca

SEQ ID NO: 292 - DNA, 19 bp, Homo sapiens
ctacctgcactataagcac

SEQ ID NO: 293 - DNA, 20 bp, Homo sapiens
ctacctgcactataagcact

SEQ ID NO: 294 - DNA, 16 bp, Homo sapiens
ctacctgcactatgag

SEQ ID NO: 295 - DNA, 17 bp, Homo sapiens
ctacctgcactatgagc

SEQ ID NO: 296 - DNA, 18 bp, Homo sapiens
ctacctgcactatgagca

SEQ ID NO: 297 - DNA, 19 bp, Homo sapiens
tcaacatcagtctgataag

SEQ ID NO: 298 - DNA, 20 bp, Homo sapiens
tcaacatcagtctgataagc

SEQ ID NO: 299 - DNA, 21 bp, Homo sapiens
tcaacatcagtctgataagct

SEQ ID NO: 300 - DNA, 14 bp, Homo sapiens
tcagccgctgtcac

SEQ ID NO: 301 - DNA, 18 bp, Homo sapiens
aggcgaaggatgacaaag

SEQ ID NO: 302 - DNA, 12 bp, Homo sapiens
ggccgtgactgg

SEQ ID NO: 303 - DNA, 13 bp, Homo sapiens
ggccgtgactgga

SEQ ID NO: 304 - DNA, 14 bp, Homo sapiens
ggccgtgactggag

SEQ ID NO: 305 - DNA, 15 bp, Homo sapiens
ggtacaatcaacggt

SEQ ID NO: 306 - DNA, 16 bp, Homo sapiens
ggtacaatcaacggtc

SEQ ID NO: 307 - DNA, 17 bp, Homo sapiens
ggtacaatcaacggtcg

SEQ ID NO: 308 - DNA, 14 bp, Homo sapiens
ctgcctgtctgtgc

SEQ ID NO: 309 - DNA, 17 bp, Homo sapiens
gtctgtcaattcatagg

SEQ ID NO: 310 - DNA, 18 bp, Homo sapiens
gtctgtcaattcataggt

SEQ ID NO: 311 - DNA, 19 bp, Homo sapiens
gtctgtcaattcataggtc

SEQ ID NO: 312 - DNA, 20 bp, Homo sapiens
gtctgtcaattcataggtca

SEQ ID NO: 313 - DNA, 21 bp, Homo sapiens
gtctgtcaattcataggtcat

SEQ ID NO: 314 - DNA, 13 bp, Homo sapiens
cacagttgccagc

SEQ ID NO: 315 - DNA, 14 bp, Homo sapiens
cacagttgccagct

SEQ ID NO: 316 - DNA, 15 bp, Homo sapiens
cacagttgccagctg

SEQ ID NO: 317 - DNA, 16 bp, Homo sapiens
cacagttgccagctga

SEQ ID NO: 318 - DNA, 16 bp, Homo sapiens
atccaatcagttcctg

SEQ ID NO: 319 - DNA, 17 bp, Homo sapiens
atccaatcagttcctga

SEQ ID NO: 320 - DNA, 18 bp, Homo sapiens
atccaatcagttcctgat

SEQ ID NO: 321 - DNA, 19 bp, Homo sapiens
atccaatcagttcctgatg

SEQ ID NO: 322 - DNA, 19 bp, Homo sapiens
acatggttagatcaagcac

SEQ ID NO: 323 - DNA, 20 bp, Homo sapiens
acatggttagatcaagcaca

SEQ ID NO: 324 - DNA, 15 bp, Homo sapiens
agaattgcgtttgga

SEQ ID NO: 325 - DNA, 16 bp, Homo sapiens
agaattgcgtttggac

SEQ ID NO: 326 - DNA, 17 bp, Homo sapiens
agaattgcgtttggaca

SEQ ID NO: 327 - DNA, 18 bp, Homo sapiens
agaattgcgtttggacaa

SEQ ID NO: 328 - DNA, 19 bp, Homo sapiens
agaattgcgtttggacaat

SEQ ID NO: 329 - DNA, 20 bp, Homo sapiens
agaattgcgtttggacaatc

SEQ ID NO: 330 - DNA, 17 bp, Homo sapiens
acagttcttcaactggc

SEQ ID NO: 331 - DNA, 18 bp, Homo sapiens
acagttcttcaactggca

SEQ ID NO: 332 - DNA, 16 bp, Homo sapiens
aaagtgtcagatacgg

SEQ ID NO: 333 - DNA, 17 bp, Homo sapiens
aaagtgtcagatacggt

SEQ ID NO: 334 - DNA, 18 bp, Homo sapiens
aaagtgtcagatacggtg

SEQ ID NO: 335 - DNA, 19 bp, Homo sapiens
aaagtgtcagatacggtgt

SEQ ID NO: 336 - DNA, 18 bp, Homo sapiens
gaaacccagcagacaatg

SEQ ID NO: 337 - DNA, 16 bp, Homo sapiens
gagacccagtagccag

SEQ ID NO: 338 - DNA, 19 bp, Homo sapiens
ggggtatttgacaaactga

SEQ ID NO: 339 - DNA, 20 bp, Homo sapiens
ggggtatttgacaaactgac

SEQ ID NO: 340 - DNA, 21 bp, Homo sapiens
ggggtatttgacaaactgaca

SEQ ID NO: 341 - DNA, 19 bp, Homo sapiens
taaacggaaccactagtga

SEQ ID NO: 342 - DNA, 20 bp, Homo sapiens
taaacggaaccactagtgac

SEQ ID NO: 343 - DNA, 21 bp, Homo sapiens
taaacggaaccactagtgact

SEQ ID NO: 344 - DNA, 17 bp, Homo sapiens
ggaaatccctggcaatg

SEQ ID NO: 345 - DNA, 17 bp, Homo sapiens
ggtaatccctggcaatg

SEQ ID NO: 346 - DNA, 17 bp, Homo sapiens
ctgttcctgctgaactg

SEQ ID NO: 347 - DNA, 17 bp, Homo sapiens
tcagaccgagacaagtg

SEQ ID NO: 348 - DNA, 16 bp, Homo sapiens
gcctatcctggattac

SEQ ID NO: 349 - DNA, 17 bp, Homo sapiens
gcctatcctggattact

SEQ ID NO: 350 - DNA, 18 bp, Homo sapiens
gcctatcctggattactt

SEQ ID NO: 351 - DNA, 18 bp, Homo sapiens
aacctatcctgaattact

SEQ ID NO: 352 - DNA, 19 bp, Homo sapiens
aacctatcctgaattactt

SEQ ID NO: 353 - DNA, 20 bp, Homo sapiens
aacctatcctgaattacttg

SEQ ID NO: 354 - DNA, 21 bp, Homo sapiens
aacctatcctgaattacttga

SEQ ID NO: 355 - DNA, 22 bp, Homo sapiens
aacctatcctgaattacttgaa

SEQ ID NO: 356 - DNA, 15 bp, Homo sapiens
gcggaacttagccac

SEQ ID NO: 357 - DNA, 16 bp, Homo sapiens
gcggaacttagccact

SEQ ID NO: 358 - DNA, 17 bp, Homo sapiens
gcagaacttagccactg

SEQ ID NO: 359 - DNA, 18 bp, Homo sapiens
gcagaacttagccactgt

SEQ ID NO: 360 - DNA, 17 bp, Homo sapiens
ctcaatagactgtgagc

SEQ ID NO: 361 - DNA, 18 bp, Homo sapiens
ctcaatagactgtgagct

SEQ ID NO: 362 - DNA, 19 bp, Homo sapiens
ctcaatagactgtgagctc

SEQ ID NO: 363 - DNA, 15 bp, Homo sapiens
acaggattgaggggg

SEQ ID NO: 364 - DNA, 15 bp, Homo sapiens
aagcggtttaccatc

SEQ ID NO: 365 - DNA, 16 bp, Homo sapiens
aagcggtttaccatcc

SEQ ID NO: 366 - DNA, 17 bp, Homo sapiens
aagcggtttaccatccc

SEQ ID NO: 367 - DNA, 15 bp, Homo sapiens
atgtatgtgggacgg

SEQ ID NO: 368 - DNA, 16 bp, Homo sapiens
atgtatgtgggacggt

SEQ ID NO: 369 - DNA, 17 bp, Homo sapiens
atgtatgtgggacggta

SEQ ID NO: 370 - DNA, 18 bp, Homo sapiens
atgtatgtgggacggtaa

SEQ ID NO: 371 - DNA, 19 bp, Homo sapiens
atgtatgtgggacggtaaa

SEQ ID NO: 372 - DNA, 18 bp, Homo sapiens
aaccgatttcagatggtg

SEQ ID NO: 373 - DNA, 20 bp, Homo sapiens
aacactgatttcaaatggtg

SEQ ID NO: 374 - DNA, 21 bp, Homo sapiens
aacactgatttcaaatggtgc

SEQ ID NO: 375 - DNA, 22 bp, Homo sapiens
aacactgatttcaaatggtgct

SEQ ID NO: 376 - DNA, 18 bp, Homo sapiens
accgatttcaaatggtgc

SEQ ID NO: 377 - DNA, 19 bp, Homo sapiens
accgatttcaaatggtgct

SEQ ID NO: 378 - DNA, 20 bp, Homo sapiens
accgatttcaaatggtgcta

SEQ ID NO: 379 - DNA, 19 bp, Homo sapiens
gctttgacaatactattgc

SEQ ID NO: 380 - DNA, 20 bp, Homo sapiens
gctttgacaatactattgca

SEQ ID NO: 381 - DNA, 18 bp, Homo sapiens
tcaccaaaacatggaagc

SEQ ID NO: 382 - DNA, 19 bp, Homo sapiens
tcaccaaaacatggaagca

SEQ ID NO: 383 - DNA, 20 bp, Homo sapiens
tcaccaaaacatggaagcac

SEQ ID NO: 384 - DNA, 17 bp, Homo sapiens
aaagcaagtacatccac

SEQ ID NO: 385 - DNA, 18 bp, Homo sapiens
aaagcaagtacatccacg

SEQ ID NO: 386 - DNA, 19 bp, Homo sapiens
aaagcaagtacatccacgt

SEQ ID NO: 387 - DNA, 20 bp, Homo sapiens
ctactaaaacatggaagcac

SEQ ID NO: 388 - DNA, 21 bp, Homo sapiens
ctactaaaacatggaagcact

SEQ ID NO: 389 - DNA, 16 bp, Homo sapiens
agaaagcacttccatg

SEQ ID NO: 390 - DNA, 17 bp, Homo sapiens
agaaagcacttccatgt

SEQ ID NO: 391 - DNA, 18 bp, Homo sapiens
agaaagcacttccatgtt

SEQ ID NO: 392 - DNA, 19 bp, Homo sapiens
agaaagcacttccatgtta

SEQ ID NO: 393 - DNA, 20 bp, Homo sapiens
agaaagcacttccatgttaa

SEQ ID NO: 394 - DNA, 21 bp, Homo sapiens
agaaagcacttccatgttaaa

SEQ ID NO: 395 - DNA, 15 bp, Homo sapiens
ccactgaaacatgga

SEQ ID NO: 396 - DNA, 16 bp, Homo sapiens
ccactgaaacatggaa

SEQ ID NO: 397 - DNA, 17 bp, Homo sapiens
ccactgaaacatggaag

SEQ ID NO: 398 - DNA, 14 bp, Homo sapiens
cagcaggtaccccc

SEQ ID NO: 399 - DNA, 19 bp, Homo sapiens
acactcaaacatggaagca

SEQ ID NO: 400 - DNA, 15 bp, Homo sapiens
gctgcaaacatccga

SEQ ID NO: 401 - DNA, 16 bp, Homo sapiens
gctgcaaacatccgac

SEQ ID NO: 402 - DNA, 15 bp, Homo sapiens
cttccagtcgaggat

SEQ ID NO: 403 - DNA, 16 bp, Homo sapiens
cttccagtcgaggatg

SEQ ID NO: 404 - DNA, 17 bp, Homo sapiens
cttccagtcgaggatgt

SEQ ID NO: 405 - DNA, 16 bp, Homo sapiens
agctgagtgtaggatg

SEQ ID NO: 406 - DNA, 17 bp, Homo sapiens
agctgagtgtaggatgt

SEQ ID NO: 407 - DNA, 18 bp, Homo sapiens
agctgagtgtaggatgtt

SEQ ID NO: 408 - DNA, 19 bp, Homo sapiens
agctgagtgtaggatgttt

SEQ ID NO: 409 - DNA, 20 bp, Homo sapiens
agctgagtgtaggatgttta

SEQ ID NO: 410 - DNA, 17 bp, Homo sapiens
gctgagagtgtaggatg

SEQ ID NO: 411 - DNA, 18 bp, Homo sapiens
gctgagagtgtaggatgt

SEQ ID NO: 412 - DNA, 14 bp, Homo sapiens
cttccagtcgggga

SEQ ID NO: 413 - DNA, 15 bp, Homo sapiens
cttccagtcggggat

SEQ ID NO: 414 - DNA, 16 bp, Homo sapiens
gctgtaaacatccgac

SEQ ID NO: 415 - DNA, 17 bp, Homo sapiens
gctgtaaacatccgact

SEQ ID NO: 416 - DNA, 18 bp, Homo sapiens
gctgtaaacatccgactg

SEQ ID NO: 417 - DNA, 20 bp, Homo sapiens
tccagtcaaggatgtttaca

SEQ ID NO: 418 - DNA, 14 bp, Homo sapiens
cagctatgccagca

SEQ ID NO: 419 - DNA, 15 bp, Homo sapiens
cagctatgccagcat

SEQ ID NO: 420 - DNA, 16 bp, Homo sapiens
cagctatgccagcatc

SEQ ID NO: 421 - DNA, 17 bp, Homo sapiens
cagctatgccagcatct

SEQ ID NO: 422 - DNA, 18 bp, Homo sapiens
cagctatgccagcatctt

SEQ ID NO: 423 - DNA, 19 bp, Homo sapiens
gcaacttagtaatgtgcaa

SEQ ID NO: 424 - DNA, 20 bp, Homo sapiens
gcaacttagtaatgtgcaat

SEQ ID NO: 425 - DNA, 21 bp, Homo sapiens
gcaacttagtaatgtgcaata

SEQ ID NO: 426 - DNA, 14 bp, Homo sapiens
ttcgccctctcaac

SEQ ID NO: 427 - DNA, 15 bp, Homo sapiens
ttcgccctctcaacc

SEQ ID NO: 428 - DNA, 16 bp, Homo sapiens
ttcgccctctcaaccc

SEQ ID NO: 429 - DNA, 13 bp, Homo sapiens
agaggtcgaccgt

SEQ ID NO: 430 - DNA, 14 bp, Homo sapiens
agaggtcgaccgtg

SEQ ID NO: 431 - DNA, 15 bp, Homo sapiens
agaggtcgaccgtgt

SEQ ID NO: 432 - DNA, 16 bp, Homo sapiens
agaggtcgaccgtgta

SEQ ID NO: 433 - DNA, 17 bp, Homo sapiens
agaggtcgaccgtgtaa

SEQ ID NO: 434 - DNA, 18 bp, Homo sapiens
agaggtcgaccgtgtaat

SEQ ID NO: 435 - DNA, 14 bp, Homo sapiens
ccagcagcacctgg

SEQ ID NO: 436 - DNA, 15 bp, Homo sapiens
ccagcagcacctggg

SEQ ID NO: 437 - DNA, 16 bp, Homo sapiens
ccagcagcacctgggg

SEQ ID NO: 438 - DNA, 16 bp, Homo sapiens
acaccaatgccctagg

SEQ ID NO: 439 - DNA, 16 bp, Homo sapiens
acacttactggacacc

SEQ ID NO: 440 - DNA, 17 bp, Homo sapiens
acacttactggacacct

SEQ ID NO: 441 - DNA, 18 bp, Homo sapiens
acacttactggacaccta

SEQ ID NO: 442 - DNA, 19 bp, Homo sapiens
acacttactggacacctac

SEQ ID NO: 443 - DNA, 20 bp, Homo sapiens
acacttactggacacctact

SEQ ID NO: 444 - DNA, 13 bp, Homo sapiens
ctggaggaagggc

SEQ ID NO: 445 - DNA, 14 bp, Homo sapiens
ctggaggaagggcc

SEQ ID NO: 446 - DNA, 16 bp, Homo sapiens
acggaagggcagagag

SEQ ID NO: 447 - DNA, 17 bp, Homo sapiens
acggaagggcagagagg

SEQ ID NO: 448 - DNA, 18 bp, Homo sapiens
acggaagggcagagaggg

SEQ ID NO: 449 - DNA, 16 bp, Homo sapiens
aaagaggttaaccagg

SEQ ID NO: 450 - DNA, 17 bp, Homo sapiens
aaagaggttaaccaggt

SEQ ID NO: 451 - DNA, 18 bp, Homo sapiens
aaagaggttaaccaggtg

SEQ ID NO: 452 - DNA, 19 bp, Homo sapiens
aaagaggttaaccaggtgt

SEQ ID NO: 453 - DNA, 19 bp, Homo sapiens
caatgcaactacaatgcac

SEQ ID NO: 454 - DNA, 13 bp, Homo sapiens
tctctgcaggccg

SEQ ID NO: 455 - DNA, 14 bp, Homo sapiens
tctctgcaggccgt

SEQ ID NO: 456 - DNA, 15 bp, Homo sapiens
tctctgcaggccgtg

SEQ ID NO: 457 - DNA, 18 bp, Homo sapiens
ttctaggataggcccagg

SEQ ID NO: 458 - DNA, 19 bp, Homo sapiens
acatttttcgttattgctc

SEQ ID NO: 459 - DNA, 20 bp, Homo sapiens
acatttttcgttattgctct

SEQ ID NO: 460 - DNA, 21 bp, Homo sapiens
acatttttcgttattgctctt

SEQ ID NO: 461 - DNA, 22 bp, Homo sapiens
acatttttcgttattgctcttg

SEQ ID NO: 462 - DNA, 18 bp, Homo sapiens
aaaggcatcatataggag

SEQ ID NO: 463 - DNA, 19 bp, Homo sapiens
aaaggcatcatataggagc

SEQ ID NO: 464 - DNA, 20 bp, Homo sapiens
aaaggcatcatataggagct

SEQ ID NO: 465 - DNA, 21 bp, Homo sapiens
aaaggcatcatataggagctg

SEQ ID NO: 466 - DNA, 21 bp, Homo sapiens
tcaacaaaatcactgatgctg

SEQ ID NO: 467 - DNA, 22 bp, Homo sapiens
tcaacaaaatcactgatgctgg

SEQ ID NO: 468 - DNA, 23 bp, Homo sapiens
tcaacaaaatcactgatgctgga

SEQ ID NO: 469 - DNA, 16 bp, Homo sapiens
tgagctcctggaggac

SEQ ID NO: 470 - DNA, 20 bp, Homo sapiens
ggctataaagtaactgagac

SEQ ID NO: 471 - DNA, 21 bp, Homo sapiens
ggctataaagtaactgagacg

SEQ ID NO: 472 - DNA, 15 bp, Homo sapiens
gacgggtgcgatttc

SEQ ID NO: 473 - DNA, 16 bp, Homo sapiens
gacgggtgcgatttct

SEQ ID NO: 474 - DNA, 15 bp, Homo sapiens
gccctggactaggag

SEQ ID NO: 475 - DNA, 16 bp, Homo sapiens
gccctggactaggagt

SEQ ID NO: 476 - DNA, 14 bp, Homo sapiens
agaggcaggcatgc

SEQ ID NO: 477 - DNA, 15 bp, Homo sapiens
agaggcaggcatgcg

SEQ ID NO: 478 - DNA, 19 bp, Homo sapiens
aacaaccagctaagacact

SEQ ID NO: 479 - DNA, 20 bp, Homo sapiens
aacaaccagctaagacactg

SEQ ID NO: 480 - DNA, 21 bp, Homo sapiens
aacaaccagctaagacactgc

SEQ ID NO: 481 - DNA, 17 bp, Homo sapiens
caatcagctaatgacac

SEQ ID NO: 482 - DNA, 18 bp, Homo sapiens
caatcagctaatgacact

SEQ ID NO: 483 - DNA, 19 bp, Homo sapiens
caatcagctaatgacactg

SEQ ID NO: 484 - DNA, 18 bp, Homo sapiens
gcaatcagctaactacac

SEQ ID NO: 485 - DNA, 19 bp, Homo sapiens
gcaatcagctaactacact

SEQ ID NO: 486 - DNA, 15 bp, Homo sapiens
gtacccctggagatt

SEQ ID NO: 487 - DNA, 15 bp, Homo sapiens
ctcacacctaggttc

SEQ ID NO: 488 - DNA, 16 bp, Homo sapiens
ctcacacctaggttcc

SEQ ID NO: 489 - DNA, 17 bp, Homo sapiens
ctcacacctaggttcca

SEQ ID NO: 490 - DNA, 18 bp, Homo sapiens
ctcacacctaggttccaa

SEQ ID NO: 491 - DNA, 17 bp, Homo sapiens
ttacagatggataccgt

SEQ ID NO: 492 - DNA, 18 bp, Homo sapiens
ttacagatggataccgtg

SEQ ID NO: 493 - DNA, 19 bp, Homo sapiens
ttacagatggataccgtgc

SEQ ID NO: 494 - DNA, 18 bp, Homo sapiens
ataaggatttttaggggc

SEQ ID NO: 495 - DNA, 19 bp, Homo sapiens
ataaggatttttaggggca

SEQ ID NO: 496 - DNA, 20 bp, Homo sapiens
ataaggatttttaggggcat

SEQ ID NO: 497 - DNA, 21 bp, Homo sapiens
ataaggatttttaggggcatt

SEQ ID NO: 498 - DNA, 22 bp, Homo sapiens
ataaggatttttaggggcatta

SEQ ID NO: 499 - DNA, 18 bp, Homo sapiens
tcaccattgctaaagtgc

SEQ ID NO: 500 - DNA, 19 bp, Homo sapiens
tcaccattgctaaagtgca

SEQ ID NO: 501 - DNA, 21 bp, Homo sapiens
aaacgtggaatttcctctatg

SEQ ID NO: 502 - DNA, 19 bp, Homo sapiens
aaagatcaaccatgtatta

SEQ ID NO: 503 - DNA, 20 bp, Homo sapiens
aaagatcaaccatgtattat

SEQ ID NO: 504 - DNA, 21 bp, Homo sapiens
aaagatcaaccatgtattatt

SEQ ID NO: 505 - DNA, 16 bp, Homo sapiens
gcgaatataacacggt

SEQ ID NO: 506 - DNA, 17 bp, Homo sapiens
gcgaatataacacggtc

SEQ ID NO: 507 - DNA, 18 bp, Homo sapiens
gcgaatataacacggtcg

SEQ ID NO: 508 - DNA, 14 bp, Homo sapiens
ccaggttccacccc

SEQ ID NO: 509 - DNA, 16 bp, Homo sapiens
acactcaaaagatggc

SEQ ID NO: 510 - DNA, 17 bp, Homo sapiens
acactcaaaagatggcg

SEQ ID NO: 511 - DNA, 18 bp, Homo sapiens
acactcaaaagatggcgg

SEQ ID NO: 512 - DNA, 15 bp, Homo sapiens
acgctcaaatgtcgc

SEQ ID NO: 513 - DNA, 16 bp, Homo sapiens
acgctcaaatgtcgca

SEQ ID NO: 514 - DNA, 17 bp, Homo sapiens
acgctcaaatgtcgcag

SEQ ID NO: 515 - DNA, 16 bp, Homo sapiens
acaccccaaaatcgaa

SEQ ID NO: 516 - DNA, 17 bp, Homo sapiens
acaccccaaaatcgaag

SEQ ID NO: 517 - DNA, 13 bp, Homo sapiens
ggaaagcgccccc

SEQ ID NO: 518 - DNA, 22 bp, Homo sapiens
cacttatcaggttgtattataa

SEQ ID NO: 519 - DNA, 12 bp, Homo sapiens
tcacgcgagccg

SEQ ID NO: 520 - DNA, 13 bp, Homo sapiens
tcacgcgagccga

SEQ ID NO: 521 - DNA, 14 bp, Homo sapiens
tcacgcgagccgaa

SEQ ID NO: 522 - DNA, 19 bp, Homo sapiens
acgtggattttcctctatg

SEQ ID NO: 523 - DNA, 20 bp, Homo sapiens
acgtggattttcctctatga

SEQ ID NO: 524 - DNA, 21 bp, Homo sapiens
acgtggattttcctctatgat

SEQ ID NO: 525 - DNA, 17 bp, Homo sapiens
aacatggattttcctct

SEQ ID NO: 526 - DNA, 18 bp, Homo sapiens
aacatggattttcctcta

SEQ ID NO: 527 - DNA, 19 bp, Homo sapiens
aacatggattttcctctat

SEQ ID NO: 528 - DNA, 20 bp, Homo sapiens
aacatggattttcctctatg

SEQ ID NO: 529 - DNA, 21 bp, Homo sapiens
aacatggattttcctctatga

SEQ ID NO: 530 - DNA, 22 bp, Homo sapiens
aacatggattttcctctatgat

SEQ ID NO: 531 - DNA, 18 bp, Homo sapiens
acaaaagttgcctttgtg

SEQ ID NO: 532 - DNA, 19 bp, Homo sapiens
acaaaagttgcctttgtgt

SEQ ID NO: 533 - DNA, 17 bp, Homo sapiens
acacaggacctggagtc

SEQ ID NO: 534 - DNA, 18 bp, Homo sapiens
tacgttccatagtctacc

SEQ ID NO: 535 - DNA, 19 bp, Homo sapiens
tacgttccatagtctacca

SEQ ID NO: 536 - DNA, 18 bp, Homo sapiens
aagatgtggaccatatta

SEQ ID NO: 537 - DNA, 19 bp, Homo sapiens
aagatgtggaccatattac

SEQ ID NO: 538 - DNA, 20 bp, Homo sapiens
aagatgtggaccatattaca

SEQ ID NO: 539 - DNA, 21 bp, Homo sapiens
aagatgtggaccatattacat

SEQ ID NO: 540 - DNA, 22 bp, Homo sapiens
aagatgtggaccatattacata

SEQ ID NO: 541 - DNA, 15 bp, Homo sapiens
gcgcatgttctatgg

SEQ ID NO: 542 - DNA, 16 bp, Homo sapiens
gcgcatgttctatggt

SEQ ID NO: 543 - DNA, 17 bp, Homo sapiens
gcgcatgttctatggtc

SEQ ID NO: 544 - DNA, 14 bp, Homo sapiens
acagagagcttgcc

SEQ ID NO: 545 - DNA, 15 bp, Homo sapiens
acagagagcttgccc

SEQ ID NO: 546 - DNA, 16 bp, Homo sapiens
acagagagcttgccct

SEQ ID NO: 547 - DNA, 17 bp, Homo sapiens
acagagagcttgccctt

SEQ ID NO: 548 - DNA, 13 bp, Homo sapiens
cgaatccaccacg

SEQ ID NO: 549 - DNA, 14 bp, Homo sapiens
cgaatccaccacga

SEQ ID NO: 550 - DNA, 15 bp, Homo sapiens
cgaatccaccacgaa

SEQ ID NO: 551 - DNA, 16 bp, Homo sapiens
cgaatccaccacgaac

SEQ ID NO: 552 - DNA, 18 bp, Homo sapiens
agccacaatcaccttctg

SEQ ID NO: 553 - DNA, 19 bp, Homo sapiens
agccacaatcaccttctga

SEQ ID NO: 554 - DNA, 20 bp, Homo sapiens
agccacaatcaccttctgat

SEQ ID NO: 555 - DNA, 19 bp, Homo sapiens
tatgaacaatttctaggaa

SEQ ID NO: 556 - DNA, 20 bp, Homo sapiens
tatgaacaatttctaggaat

SEQ ID NO: 557 - DNA, 13 bp, Homo sapiens
aggggttcaccga

SEQ ID NO: 558 - DNA, 14 bp, Homo sapiens
aggggttcaccgag

SEQ ID NO: 559 - DNA, 15 bp, Homo sapiens
aggggttcaccgagc

SEQ ID NO: 560 - DNA, 14 bp, Homo sapiens
tgcaaagttgctcg

SEQ ID NO: 561 - DNA, 15 bp, Homo sapiens
tgcaaagttgctcgg

SEQ ID NO: 562 - DNA, 16 bp, Homo sapiens
tgcaaagttgctcggg

SEQ ID NO: 563 - DNA, 15 bp, Homo sapiens
aacaggccatctgtg

SEQ ID NO: 564 - DNA, 16 bp, Homo sapiens
aacaggccatctgtgt

SEQ ID NO: 565 - DNA, 17 bp, Homo sapiens
aacaggccatctgtgtt

SEQ ID NO: 566 - DNA, 18 bp, Homo sapiens
aacaggccatctgtgtta

SEQ ID NO: 567 - DNA, 19 bp, Homo sapiens
aacaggccatctgtgttat

SEQ ID NO: 568 - DNA, 20 bp, Homo sapiens
aacaggccatctgtgttata

SEQ ID NO: 569 - DNA, 21 bp, Homo sapiens
aacaggccatctgtgttatat

SEQ ID NO: 570 - DNA, 14 bp, Homo sapiens
acggctagtggacc

SEQ ID NO: 571 - DNA, 15 bp, Homo sapiens
acggctagtggacca

SEQ ID NO: 572 - DNA, 16 bp, Homo sapiens
acggctagtggaccag

SEQ ID NO: 573 - DNA, 17 bp, Homo sapiens
ggccttctgaccctaag

SEQ ID NO: 574 - DNA, 18 bp, Homo sapiens
ggccttctgaccctaagt

SEQ ID NO: 575 - DNA, 19 bp, Homo sapiens
ggccttctgaccctaagtc

SEQ ID NO: 576 - DNA, 15 bp, Homo sapiens
ggccttctgactcca

SEQ ID NO: 577 - DNA, 16 bp, Homo sapiens
ggccttctgactccaa

SEQ ID NO: 578 - DNA, 12 bp, Homo sapiens
ctgaggggcctc

SEQ ID NO: 579 - DNA, 13 bp, Homo sapiens
ctgaggggcctca

SEQ ID NO: 580 - DNA, 14 bp, Homo sapiens
ctgaggggcctcag

SEQ ID NO: 581 - DNA, 21 bp, Homo sapiens
ttcaaaacatgaattgctgct

SEQ ID NO: 582 - DNA, 22 bp, Homo sapiens
ttcaaaacatgaattgctgctg

SEQ ID NO: 583 - DNA, 12 bp, Homo sapiens
ggcggacacgac

SEQ ID NO: 584 - DNA, 13 bp, Homo sapiens
ggcggacacgaca

SEQ ID NO: 585 - DNA, 14 bp, Homo sapiens
ggcggacacgacat

SEQ ID NO: 586 - DNA, 15 bp, Homo sapiens
ggcggacacgacatt

SEQ ID NO: 587 - DNA, 15 bp, Homo sapiens
acggttttaccagac

SEQ ID NO: 588 - DNA, 16 bp, Homo sapiens
acggttttaccagaca

SEQ ID NO: 589 - DNA, 17 bp, Homo sapiens
acggttttaccagacag

SEQ ID NO: 590 - DNA, 18 bp, Homo sapiens
acggttttaccagacagt

SEQ ID NO: 591 - DNA, 19 bp, Homo sapiens
acggttttaccagacagta

SEQ ID NO: 592 - DNA, 20 bp, Homo sapiens
acggttttaccagacagtat

SEQ ID NO: 593 - DNA, 13 bp, Homo sapiens
tgcatgacggcct

SEQ ID NO: 594 - DNA, 14 bp, Homo sapiens
tgcatgacggcctg

SEQ ID NO: 595 - DNA, 15 bp, Homo sapiens
tgcatgacggcctgc

SEQ ID NO: 596 - DNA, 13 bp, Homo sapiens
ccacccaatgacc

SEQ ID NO: 597 - DNA, 14 bp, Homo sapiens
ccacccaatgacct

SEQ ID NO: 598 - DNA, 15 bp, Homo sapiens
ccacccaatgaccta

SEQ ID NO: 599 - DNA, 16 bp, Homo sapiens
ccacccaatgacctac

SEQ ID NO: 600 - DNA, 17 bp, Homo sapiens
ccacccaatgacctact

SEQ ID NO: 601 - DNA, 15 bp, Homo sapiens
agacatggaggagcc

SEQ ID NO: 602 - DNA, 16 bp, Homo sapiens
agacatggaggagcca

SEQ ID NO: 603 - DNA, 17 bp, Homo sapiens
agacatggaggagccat

SEQ ID NO: 604 - DNA, 13 bp, Homo sapiens
acaccgaggagcc

SEQ ID NO: 605 - DNA, 14 bp, Homo sapiens
acaccgaggagccc

SEQ ID NO: 606 - DNA, 16 bp, Homo sapiens
atgggacatcctacat

SEQ ID NO: 607 - DNA, 17 bp, Homo sapiens
atgggacatcctacata

SEQ ID NO: 608 - DNA, 18 bp, Homo sapiens
atgggacatcctacatat

SEQ ID NO: 609 - DNA, 19 bp, Homo sapiens
atgggacatcctacatatg

SEQ ID NO: 610 - DNA, 20 bp, Homo sapiens
atgggacatcctacatatgc

SEQ ID NO: 611 - DNA, 17 bp, Homo sapiens
accagctaacaatacac

SEQ ID NO: 612 - DNA, 18 bp, Homo sapiens
accagctaacaatacact

SEQ ID NO: 613 - DNA, 19 bp, Homo sapiens
accagctaacaatacactg

SEQ ID NO: 614 - DNA, 20 bp, Homo sapiens
accagctaacaatacactgc

SEQ ID NO: 615 - DNA, 17 bp, Homo sapiens
tattaggaacacatcgc

SEQ ID NO: 616 - DNA, 18 bp, Homo sapiens
tattaggaacacatcgca

SEQ ID NO: 617 - DNA, 19 bp, Homo sapiens
tattaggaacacatcgcaa

SEQ ID NO: 618 - DNA, 20 bp, Homo sapiens
tattaggaacacatcgcaaa

SEQ ID NO: 619 - DNA, 21 bp, Homo sapiens
tattaggaacacatcgcaaaa

SEQ ID NO: 620 - DNA, 22 bp, Homo sapiens
tattaggaacacatcgcaaaaa

SEQ ID NO: 621 - DNA, 18 bp, Homo sapiens
aaactcagtaatggtaac

SEQ ID NO: 622 - DNA, 19 bp, Homo sapiens
aaactcagtaatggtaacg

SEQ ID NO: 623 - DNA, 20 bp, Homo sapiens
aaactcagtaatggtaacgg

SEQ ID NO: 624 - DNA, 16 bp, Homo sapiens
gtctcagtttcctctg

SEQ ID NO: 625 - DNA, 17 bp, Homo sapiens
gtctcagtttcctctgc

SEQ ID NO: 626 - DNA, 16 bp, Homo sapiens
cttctttgcagatgag

SEQ ID NO: 627 - DNA, 17 bp, Homo sapiens
cttctttgcagatgaga

SEQ ID NO: 628 - DNA, 18 bp, Homo sapiens
cttctttgcagatgagac

SEQ ID NO: 629 - DNA, 19 bp, Homo sapiens
cttctttgcagatgagact

SEQ ID NO: 630 - DNA, 13 bp, Homo sapiens
cgaactcaccacg

SEQ ID NO: 631 - DNA, 14 bp, Homo sapiens
cgaactcaccacgg

SEQ ID NO: 632 - DNA, 15 bp, Homo sapiens
cgaactcaccacgga

SEQ ID NO: 633 - DNA, 14 bp, Homo sapiens
agaggagagccgtg

SEQ ID NO: 634 - DNA, 15 bp, Homo sapiens
agaggagagccgtgt

SEQ ID NO: 635 - DNA, 16 bp, Homo sapiens
agaggagagccgtgta

SEQ ID NO: 636 - DNA, 17 bp, Homo sapiens
agaggagagccgtgtat

SEQ ID NO: 637 - DNA, 18 bp, Homo sapiens
agaggagagccgtgtatg

SEQ ID NO: 638 - DNA, 15 bp, Homo sapiens
gaattcatcacggcc

SEQ ID NO: 639 - DNA, 16 bp, Homo sapiens
gaattcatcacggcca

SEQ ID NO: 640 - DNA, 17 bp, Homo sapiens
gaattcatcacggccag

SEQ ID NO: 641 - DNA, 17 bp, Homo sapiens
ttgagagtgccattatc

SEQ ID NO: 642 - DNA, 18 bp, Homo sapiens
ttgagagtgccattatct

SEQ ID NO: 643 - DNA, 19 bp, Homo sapiens
ttgagagtgccattatctg

SEQ ID NO: 644 - DNA, 20 bp, Homo sapiens
ttgagagtgccattatctgg

SEQ ID NO: 645 - DNA, 15 bp, Homo sapiens
gctgccgtatatgtg

SEQ ID NO: 646 - DNA, 16 bp, Homo sapiens
gctgccgtatatgtga

SEQ ID NO: 647 - DNA, 17 bp, Homo sapiens
gctgccgtatatgtgat

SEQ ID NO: 648 - DNA, 18 bp, Homo sapiens
gctgccgtatatgtgatg

SEQ ID NO: 649 - DNA, 14 bp, Homo sapiens
cagcatggagtcct

SEQ ID NO: 650 - DNA, 15 bp, Homo sapiens
cagcatggagtcctc

SEQ ID NO: 651 - DNA, 16 bp, Homo sapiens
cagcatggagtcctcc

SEQ ID NO: 652 - DNA, 15 bp, Homo sapiens
tcctcatggaagggt

SEQ ID NO: 653 - DNA, 16 bp, Homo sapiens
tcctcatggaagggtt

SEQ ID NO: 654 - DNA, 17 bp, Homo sapiens
tcctcatggaagggttc

SEQ ID NO: 655 - DNA, 16 bp, Homo sapiens
aagaatcttgtcccgc

SEQ ID NO: 656 - DNA, 17 bp, Homo sapiens
aagaatcttgtcccgca

SEQ ID NO: 657 - DNA, 18 bp, Homo sapiens
aagaatcttgtcccgcag

SEQ ID NO: 658 - DNA, 17 bp, Homo sapiens
aatgaaagcctaccatg

SEQ ID NO: 659 - DNA, 18 bp, Homo sapiens
aatgaaagcctaccatgt

SEQ ID NO: 660 - DNA, 19 bp, Homo sapiens
aatgaaagcctaccatgta

SEQ ID NO: 661 - DNA, 20 bp, Homo sapiens
aatgaaagcctaccatgtac

SEQ ID NO: 662 - DNA, 21 bp, Homo sapiens
aatgaaagcctaccatgtaca

SEQ ID NO: 663 - DNA, 14 bp, Homo sapiens
aagaggtttcccgt

SEQ ID NO: 664 - DNA, 15 bp, Homo sapiens
aagaggtttcccgtg

SEQ ID NO: 665 - DNA, 16 bp, Homo sapiens
aagaggtttcccgtgt

SEQ ID NO: 666 - DNA, 17 bp, Homo sapiens
aagaggtttcccgtgta

SEQ ID NO: 667 - DNA, 18 bp, Homo sapiens
aagaggtttcccgtgtat

SEQ ID NO: 668 - DNA, 19 bp, Homo sapiens
aagaggtttcccgtgtatg

SEQ ID NO: 669 - DNA, 16 bp, Homo sapiens
aaagaagtgcaccatg

SEQ ID NO: 670 - DNA, 17 bp, Homo sapiens
aaagaagtgcaccatgt

SEQ ID NO: 671 - DNA, 18 bp, Homo sapiens
aaagaagtgcaccatgtt

SEQ ID NO: 672 - DNA, 19 bp, Homo sapiens
aaagaagtgcaccatgttt

SEQ ID NO: 673 - DNA, 20 bp, Homo sapiens
aaagaagtgcaccatgtttg

SEQ ID NO: 674 - DNA, 15 bp, Homo sapiens
gagattggccatgta

SEQ ID NO: 675 - DNA, 16 bp, Homo sapiens
gagattggccatgtaa

SEQ ID NO: 676 - DNA, 17 bp, Homo sapiens
gagattggccatgtaat

SEQ ID NO: 677 - DNA, 15 bp, Homo sapiens
acaaaccacagtgtg

SEQ ID NO: 678 - DNA, 16 bp, Homo sapiens
acaaaccacagtgtgc

SEQ ID NO: 679 - DNA, 17 bp, Homo sapiens
acaaaccacagtgtgct

SEQ ID NO: 680 - DNA, 18 bp, Homo sapiens
acaaaccacagtgtgctg

SEQ ID NO: 681 - DNA, 13 bp, Homo sapiens
gaaaaacgccccc

SEQ ID NO: 682 - DNA, 14 bp, Homo sapiens
gaaaaacgccccct

SEQ ID NO: 683 - DNA, 15 bp, Homo sapiens
gaaaaacgccccctg

SEQ ID NO: 684 - DNA, 17 bp, Homo sapiens
ttaaacatcactgcaag

SEQ ID NO: 685 - DNA, 18 bp, Homo sapiens
ttaaacatcactgcaagt

SEQ ID NO: 686 - DNA, 19 bp, Homo sapiens
ttaaacatcactgcaagtc

SEQ ID NO: 687 - DNA, 20 bp, Homo sapiens
ttaaacatcactgcaagtct

SEQ ID NO: 688 - DNA, 21 bp, Homo sapiens
ttaaacatcactgcaagtctt

SEQ ID NO: 689 - DNA, 22 bp, Homo sapiens
ttaaacatcactgcaagtctta

SEQ ID NO: 690 - DNA, 23 bp, Homo sapiens
ttaaacatcactgcaagtcttaa

SEQ ID NO: 691 - DNA, 14 bp, Homo sapiens
cagaatccttgccc

SEQ ID NO: 692 - DNA, 15 bp, Homo sapiens
cagaatccttgccca

SEQ ID NO: 693 - DNA, 16 bp, Homo sapiens
cagaatccttgcccag

SEQ ID NO: 694 - DNA, 14 bp, Homo sapiens
tctcacccagggac

SEQ ID NO: 695 - DNA, 15 bp, Homo sapiens
tctcacccagggaca

SEQ ID NO: 696 - DNA, 16 bp, Homo sapiens
tctcacccagggacaa

SEQ ID NO: 697 - DNA, 17 bp, Homo sapiens
tctcacccagggacaaa

SEQ ID NO: 698 - DNA, 15 bp, Homo sapiens
tagcacccagatagc

SEQ ID NO: 699 - DNA, 16 bp, Homo sapiens
tagcacccagatagca

SEQ ID NO: 700 - DNA, 17 bp, Homo sapiens
tagcacccagatagcaa

SEQ ID NO: 701 - DNA, 18 bp, Homo sapiens
tagcacccagatagcaag

SEQ ID NO: 702 - DNA, 15 bp, Homo sapiens
ctgcagaactgttcc

SEQ ID NO: 703 - DNA, 16 bp, Homo sapiens
ctgcagaactgttccc

SEQ ID NO: 704 - DNA, 16 bp, Homo sapiens
atagagtgcagaccag

SEQ ID NO: 705 - DNA, 17 bp, Homo sapiens
atagagtgcagaccagg

SEQ ID NO: 706 - DNA, 18 bp, Homo sapiens
atagagtgcagaccaggg

SEQ ID NO: 707 - DNA, 14 bp, Homo sapiens
gaggaaaccagcaa

SEQ ID NO: 708 - DNA, 15 bp, Homo sapiens
gaggaaaccagcaag

SEQ ID NO: 709 - DNA, 16 bp, Homo sapiens
gaggaaaccagcaagt

SEQ ID NO: 710 - DNA, 17 bp, Homo sapiens
gaggaaaccagcaagtg

SEQ ID NO: 711 - DNA, 16 bp, Homo sapiens
tctactcagaagggtg

SEQ ID NO: 712 - DNA, 17 bp, Homo sapiens
tctactcagaagggtgc

SEQ ID NO: 713 - DNA, 16 bp, Homo sapiens
ttcactccaaaaggtg

SEQ ID NO: 714 - DNA, 17 bp, Homo sapiens
ttcactccaaaaggtgc

SEQ ID NO: 715 - DNA, 18 bp, Homo sapiens
ttcactccaaaaggtgca

SEQ ID NO: 716 - DNA, 16 bp, Homo sapiens
tctactccaaaaggct

SEQ ID NO: 717 - DNA, 17 bp, Homo sapiens
tctactccaaaaggcta

SEQ ID NO: 718 - DNA, 18 bp, Homo sapiens
tctactccaaaaggctac

SEQ ID NO: 719 - DNA, 19 bp, Homo sapiens
tctactccaaaaggctaca

SEQ ID NO: 720 - DNA, 20 bp, Homo sapiens
tctactccaaaaggctacaa

SEQ ID NO: 721 - DNA, 21 bp, Homo sapiens
tctactccaaaaggctacaat

SEQ ID NO: 722 - DNA, 15 bp, Homo sapiens
tctacccacagacgt

SEQ ID NO: 723 - DNA, 16 bp, Homo sapiens
tctacccacagacgta

SEQ ID NO: 724 - DNA, 17 bp, Homo sapiens
tctacccacagacgtac

SEQ ID NO: 725 - DNA, 15 bp, Homo sapiens
tgtgattgccactct

SEQ ID NO: 726 - DNA, 16 bp, Homo sapiens
tgtgattgccactctc

SEQ ID NO: 727 - DNA, 17 bp, Homo sapiens
tgtgattgccactctcc

SEQ ID NO: 728 - DNA, 18 bp, Homo sapiens
tgtgattgccactctcct

SEQ ID NO: 729 - DNA, 15 bp, Homo sapiens
tgactgcagagcaaa

SEQ ID NO: 730 - DNA, 16 bp, Homo sapiens
tgactgcagagcaaaa

SEQ ID NO: 731 - DNA, 17 bp, Homo sapiens
tgactgcagagcaaaag

SEQ ID NO: 732 - DNA, 18 bp, Homo sapiens
tgactgcagagcaaaaga

SEQ ID NO: 733 - DNA, 15 bp, Homo sapiens
gacctcagctatgac

SEQ ID NO: 734 - DNA, 16 bp, Homo sapiens
gacctcagctatgaca

SEQ ID NO: 735 - DNA, 17 bp, Homo sapiens
gacctcagctatgacag

SEQ ID NO: 736 - DNA, 14 bp, Homo sapiens
gaaagtgccctcaa

SEQ ID NO: 737 - DNA, 15 bp, Homo sapiens
gaaagtgccctcaag

SEQ ID NO: 738 - DNA, 16 bp, Homo sapiens
gaaagtgccctcaagg

SEQ ID NO: 739 - DNA, 17 bp, Homo sapiens
ataaatgacacctccct

SEQ ID NO: 740 - DNA, 18 bp, Homo sapiens
ataaatgacacctccctg

SEQ ID NO: 741 - DNA, 19 bp, Homo sapiens
ataaatgacacctccctgt

SEQ ID NO: 742 - DNA, 20 bp, Homo sapiens
ataaatgacacctccctgtg

SEQ ID NO: 743 - DNA, 16 bp, Homo sapiens
ctactcacagaagtgt

SEQ ID NO: 744 - DNA, 17 bp, Homo sapiens
ctactcacagaagtgtc

SEQ ID NO: 745 - DNA, 18 bp, Homo sapiens
ctactcacagaagtgtca

SEQ ID NO: 746 - DNA, 19 bp, Homo sapiens
ctactcacagaagtgtcaa

SEQ ID NO: 747 - DNA, 20 bp, Homo sapiens
ctactcacagaagtgtcaat

SEQ ID NO: 748 - DNA, 15 bp, Homo sapiens
acgctccaaaagaag

SEQ ID NO: 749 - DNA, 16 bp, Homo sapiens
acgctccaaaagaagg

SEQ ID NO: 750 - DNA, 17 bp, Homo sapiens
acgctccaaaagaaggc

SEQ ID NO: 751 - DNA, 16 bp, Homo sapiens
cagaaagtgctttctt

SEQ ID NO: 752 - DNA, 17 bp, Homo sapiens
cagaaagtgctttcttt

SEQ ID NO: 753 - DNA, 18 bp, Homo sapiens
cagaaagtgctttctttt

SEQ ID NO: 754 - DNA, 19 bp, Homo sapiens
cagaaagtgctttcttttg

SEQ ID NO: 755 - DNA, 15 bp, Homo sapiens
accctctgaaaggaa

SEQ ID NO: 756 - DNA, 16 bp, Homo sapiens
accctctgaaaggaag

SEQ ID NO: 757 - DNA, 17 bp, Homo sapiens
accctctgaaaggaagc

SEQ ID NO: 758 - DNA, 17 bp, Homo sapiens
aaagtgcttcttacctc

SEQ ID NO: 759 - DNA, 18 bp, Homo sapiens
aaagtgcttcttacctcc

SEQ ID NO: 760 - DNA, 19 bp, Homo sapiens
aaagtgcttcttacctcca

SEQ ID NO: 761 - DNA, 15 bp, Homo sapiens
agacagtgcttccat

SEQ ID NO: 762 - DNA, 16 bp, Homo sapiens
agacagtgcttccatc

SEQ ID NO: 763 - DNA, 17 bp, Homo sapiens
agacagtgcttccatct

SEQ ID NO: 764 - DNA, 18 bp, Homo sapiens
agacagtgcttccatcta

SEQ ID NO: 765 - DNA, 19 bp, Homo sapiens
agacagtgcttccatctag

SEQ ID NO: 766 - DNA, 17 bp, Homo sapiens
aacactctaaagggatg

SEQ ID NO: 767 - DNA, 18 bp, Homo sapiens
aacactctaaagggatgc

SEQ ID NO: 768 - DNA, 19 bp, Homo sapiens
aacactctaaagggatgca

SEQ ID NO: 769 - DNA, 17 bp, Homo sapiens
acactctaaaaggatgc

SEQ ID NO: 770 - DNA, 18 bp, Homo sapiens
acactctaaaaggatgca

SEQ ID NO: 771 - DNA, 19 bp, Homo sapiens
acactctaaaaggatgcac

SEQ ID NO: 772 - DNA, 14 bp, Homo sapiens
tccagcaaagggaa

SEQ ID NO: 773 - DNA, 15 bp, Homo sapiens
tccagcaaagggaag

SEQ ID NO: 774 - DNA, 16 bp, Homo sapiens
tccagcaaagggaagc

SEQ ID NO: 775 - DNA, 15 bp, Homo sapiens
aaagggcttcccttt

SEQ ID NO: 776 - DNA, 16 bp, Homo sapiens
aaagggcttccctttg

SEQ ID NO: 777 - DNA, 17 bp, Homo sapiens
aaagggcttccctttgc

SEQ ID NO: 778 - DNA, 15 bp, Homo sapiens
acctctaaaggggag

SEQ ID NO: 779 - DNA, 16 bp, Homo sapiens
acctctaaaggggagc

SEQ ID NO: 780 - DNA, 17 bp, Homo sapiens
acctctaaaggggagcg

SEQ ID NO: 781 - DNA, 16 bp, Homo sapiens
cactctaaagagaagc

SEQ ID NO: 782 - DNA, 17 bp, Homo sapiens
cactctaaagagaagcg

SEQ ID NO: 783 - DNA, 18 bp, Homo sapiens
cactctaaagagaagcgc

SEQ ID NO: 784 - DNA, 15 bp, Homo sapiens
cagaaagtgcttccc

SEQ ID NO: 785 - DNA, 16 bp, Homo sapiens
cagaaagtgcttccct

SEQ ID NO: 786 - DNA, 17 bp, Homo sapiens
cagaaagtgcttccctc

SEQ ID NO: 787 - DNA, 14 bp, Homo sapiens
gctccaaagggaag

SEQ ID NO: 788 - DNA, 15 bp, Homo sapiens
gctccaaagggaagc

SEQ ID NO: 789 - DNA, 15 bp, Homo sapiens
acactctgaagggaa

SEQ ID NO: 790 - DNA, 16 bp, Homo sapiens
acactctgaagggaag

SEQ ID NO: 791 - DNA, 17 bp, Homo sapiens
acactctgaagggaagc

SEQ ID NO: 792 - DNA, 17 bp, Homo sapiens
tcctctaaagagaagcg

SEQ ID NO: 793 - DNA, 18 bp, Homo sapiens
tcctctaaagagaagcgc

SEQ ID NO: 794 - DNA, 16 bp, Homo sapiens
agagaaagtgcttccc

SEQ ID NO: 795 - DNA, 17 bp, Homo sapiens
agagaaagtgcttccct

SEQ ID NO: 796 - DNA, 18 bp, Homo sapiens
agagaaagtgcttccctc

SEQ ID NO: 797 - DNA, 17 bp, Homo sapiens
gtaacactctaaaagga

SEQ ID NO: 798 - DNA, 18 bp, Homo sapiens
gtaacactctaaaaggat

SEQ ID NO: 799 - DNA, 19 bp, Homo sapiens
gtaacactctaaaaggatg

SEQ ID NO: 800 - DNA, 20 bp, Homo sapiens
gtaacactctaaaaggatgc

SEQ ID NO: 801 - DNA, 21 bp, Homo sapiens
gtaacactctaaaaggatgca

SEQ ID NO: 802 - DNA, 18 bp, Homo sapiens
aaacctctaaaaggatgc

SEQ ID NO: 803 - DNA, 19 bp, Homo sapiens
aaacctctaaaaggatgca

SEQ ID NO: 804 - DNA, 20 bp, Homo sapiens
aaacctctaaaaggatgcac

SEQ ID NO: 805 - DNA, 18 bp, Homo sapiens
atcctctaaaaagatgca

SEQ ID NO: 806 - DNA, 19 bp, Homo sapiens
atcctctaaaaagatgcac

SEQ ID NO: 807 - DNA, 20 bp, Homo sapiens
atcctctaaaaagatgcact

SEQ ID NO: 808 - DNA, 21 bp, Homo sapiens
atcctctaaaaagatgcactt

SEQ ID NO: 809 - DNA, 22 bp, Homo sapiens
atcctctaaaaagatgcacttt

SEQ ID NO: 810 - DNA, 16 bp, Homo sapiens
acactctaaagggagg

SEQ ID NO: 811 - DNA, 17 bp, Homo sapiens
acactctaaagggaggc

SEQ ID NO: 812 - DNA, 18 bp, Homo sapiens
acactctaaagggaggca

SEQ ID NO: 813 - DNA, 17 bp, Homo sapiens
acactctaaaaggaggc

SEQ ID NO: 814 - DNA, 18 bp, Homo sapiens
acactctaaaaggaggca

SEQ ID NO: 815 - DNA, 19 bp, Homo sapiens
acactctaaaaggaggcac

SEQ ID NO: 816 - DNA, 15 bp, Homo sapiens
gaaagtgctcccttt

SEQ ID NO: 817 - DNA, 16 bp, Homo sapiens
gaaagtgctccctttt

SEQ ID NO: 818 - DNA, 17 bp, Homo sapiens
gaaagtgctcccttttg

SEQ ID NO: 819 - DNA, 15 bp, Homo sapiens
acagtccaaagggaa

SEQ ID NO: 820 - DNA, 16 bp, Homo sapiens
acagtccaaagggaag

SEQ ID NO: 821 - DNA, 17 bp, Homo sapiens
acagtccaaagggaagc

SEQ ID NO: 822 - DNA, 17 bp, Homo sapiens
agaaagtacttccctct

SEQ ID NO: 823 - DNA, 18 bp, Homo sapiens
agaaagtacttccctctg

SEQ ID NO: 824 - DNA, 19 bp, Homo sapiens
agaaagtacttccctctgg

SEQ ID NO: 825 - DNA, 16 bp, Homo sapiens
ccctctaaaaggaagc

SEQ ID NO: 826 - DNA, 17 bp, Homo sapiens
ccctctaaaaggaagca

SEQ ID NO: 827 - DNA, 18 bp, Homo sapiens
ccctctaaaaggaagcac

SEQ ID NO: 828 - DNA, 17 bp, Homo sapiens
aaccctctaaaaggaag

SEQ ID NO: 829 - DNA, 18 bp, Homo sapiens
aaccctctaaaaggaagc

SEQ ID NO: 830 - DNA, 19 bp, Homo sapiens
aaccctctaaaaggaagca

SEQ ID NO: 831 - DNA, 15 bp, Homo sapiens
aacccaccaaagaga

SEQ ID NO: 832 - DNA, 16 bp, Homo sapiens
aacccaccaaagagaa

SEQ ID NO: 833 - DNA, 17 bp, Homo sapiens
aacccaccaaagagaag

SEQ ID NO: 834 - DNA, 18 bp, Homo sapiens
aacccaccaaagagaagc

SEQ ID NO: 835 - DNA, 14 bp, Homo sapiens
cagaaagggcttcc

SEQ ID NO: 836 - DNA, 15 bp, Homo sapiens
cagaaagggcttccc

SEQ ID NO: 837 - DNA, 16 bp, Homo sapiens
cagaaagggcttccct

SEQ ID NO: 838 - DNA, 15 bp, Homo sapiens
ccctcaaaaaggaag

SEQ ID NO: 839 - DNA, 16 bp, Homo sapiens
ccctcaaaaaggaagc

SEQ ID NO: 840 - DNA, 17 bp, Homo sapiens
ccctcaaaaaggaagca

SEQ ID NO: 841 - DNA, 17 bp, Homo sapiens
acactctaaagggaagc

SEQ ID NO: 842 - DNA, 18 bp, Homo sapiens
acactctaaagggaagca

SEQ ID NO: 843 - DNA, 19 bp, Homo sapiens
acactctaaagggaagcac

SEQ ID NO: 844 - DNA, 16 bp, Homo sapiens
actctaaagggaagca

SEQ ID NO: 845 - DNA, 17 bp, Homo sapiens
actctaaagggaagcac

SEQ ID NO: 846 - DNA, 18 bp, Homo sapiens
actctaaagggaagcact

SEQ ID NO: 847 - DNA, 19 bp, Homo sapiens
actctaaagggaagcactt

SEQ ID NO: 848 - DNA, 20 bp, Homo sapiens
actctaaagggaagcacttt

SEQ ID NO: 849 - DNA, 17 bp, Homo sapiens
acactctaaagggaagt

SEQ ID NO: 850 - DNA, 18 bp, Homo sapiens
acactctaaagggaagtg

SEQ ID NO: 851 - DNA, 19 bp, Homo sapiens
acactctaaagggaagtgc

SEQ ID NO: 852 - DNA, 17 bp, Homo sapiens
aacactctaaagggaac

SEQ ID NO: 853 - DNA, 18 bp, Homo sapiens
aacactctaaagggaacc

SEQ ID NO: 854 - DNA, 19 bp, Homo sapiens
aacactctaaagggaacca

SEQ ID NO: 855 - DNA, 20 bp, Homo sapiens
aacactctaaagggaaccat

SEQ ID NO: 856 - DNA, 16 bp, Homo sapiens
ccctctatagggaagc

SEQ ID NO: 857 - DNA, 17 bp, Homo sapiens
ccctctatagggaagcg

SEQ ID NO: 858 - DNA, 15 bp, Homo sapiens
actccaaagggaagc

SEQ ID NO: 859 - DNA, 16 bp, Homo sapiens
actccaaagggaagcg

SEQ ID NO: 860 - DNA, 15 bp, Homo sapiens
gagaaagtgcttccc

SEQ ID NO: 861 - DNA, 16 bp, Homo sapiens
gagaaagtgcttccct

SEQ ID NO: 862 - DNA, 17 bp, Homo sapiens
gagaaagtgcttccctt

SEQ ID NO: 863 - DNA, 18 bp, Homo sapiens
gagaaagtgcttcccttt

SEQ ID NO: 864 - DNA, 15 bp, Homo sapiens
agaaagtgcatccct

SEQ ID NO: 865 - DNA, 16 bp, Homo sapiens
agaaagtgcatccctc

SEQ ID NO: 866 - DNA, 17 bp, Homo sapiens
agaaagtgcatccctct

SEQ ID NO: 867 - DNA, 18 bp, Homo sapiens
agaaagtgcatccctctg

SEQ ID NO: 868 - DNA, 15 bp, Homo sapiens
gctctaaagggaagc

SEQ ID NO: 869 - DNA, 16 bp, Homo sapiens
gctctaaagggaagcg

SEQ ID NO: 870 - DNA, 15 bp, Homo sapiens
agaaagtgcttccct

SEQ ID NO: 871 - DNA, 16 bp, Homo sapiens
agaaagtgcttccctc

SEQ ID NO: 872 - DNA, 17 bp, Homo sapiens
agaaagtgcttccctct

SEQ ID NO: 873 - DNA, 18 bp, Homo sapiens
agaaagtgcttccctcta

SEQ ID NO: 874 - DNA, 19 bp, Homo sapiens
agaaagtgcttccctctag

SEQ ID NO: 875 - DNA, 16 bp, Homo sapiens
aacagaaagtgcttcc

SEQ ID NO: 876 - DNA, 17 bp, Homo sapiens
aacagaaagtgcttccc

SEQ ID NO: 877 - DNA, 18 bp, Homo sapiens
aacagaaagtgcttccct

SEQ ID NO: 878 - DNA, 16 bp, Homo sapiens
gcctctaaaaggaagc

SEQ ID NO: 879 - DNA, 17 bp, Homo sapiens
gcctctaaaaggaagca

SEQ ID NO: 880 - DNA, 18 bp, Homo sapiens
gcctctaaaaggaagcac

SEQ ID NO: 881 - DNA, 15 bp, Homo sapiens
aacagaaagcgcttc

SEQ ID NO: 882 - DNA, 16 bp, Homo sapiens
aacagaaagcgcttcc

SEQ ID NO: 883 - DNA, 17 bp, Homo sapiens
aacagaaagcgcttccc

SEQ ID NO: 884 - DNA, 14 bp, Homo sapiens
agaaagggcttccc

SEQ ID NO: 885 - DNA, 15 bp, Homo sapiens
agaaagggcttccct

SEQ ID NO: 886 - DNA, 16 bp, Homo sapiens
agaaagggcttccctt

SEQ ID NO: 887 - DNA, 17 bp, Homo sapiens
agaaagggcttcccttt

SEQ ID NO: 888 - DNA, 18 bp, Homo sapiens
agaaagggcttccctttg

SEQ ID NO: 889 - DNA, 21 bp, Homo sapiens
caacaaaatcactagtcttcc

SEQ ID NO: 890 - DNA, 19 bp, Homo sapiens
tcatacagctagataacca

SEQ ID NO: 891 - DNA, 20 bp, Homo sapiens
tcatacagctagataaccaa

SEQ ID NO: 892 - DNA, 21 bp, Homo sapiens
tcatacagctagataaccaaa

SEQ ID NO: 893 - DNA, 22 bp, Homo sapiens
tcatacagctagataaccaaag

SEQ ID NO: 894 - DNA, 23 bp, Homo sapiens
tcatacagctagataaccaaaga

SEQ ID NO: 895 - DNA, 17 bp, Homo sapiens
actttcggttatctagc

SEQ ID NO: 896 - DNA, 18 bp, Homo sapiens
actttcggttatctagct

SEQ ID NO: 897 - DNA, 19 bp, Homo sapiens
actttcggttatctagctt

SEQ ID NO: 898 - DNA, 20 bp, Homo sapiens
actttcggttatctagcttt

SEQ ID NO: 899 - DNA, 21 bp, Homo sapiens
actttcggttatctagcttta

SEQ ID NO: 900 - DNA, 14 bp, Homo sapiens
caggccgggacaag

SEQ ID NO: 901 - DNA, 15 bp, Homo sapiens
caggccgggacaagt

SEQ ID NO: 902 - DNA, 16 bp, Homo sapiens
caggccgggacaagtg

SEQ ID NO: 903 - DNA, 16 bp, Homo sapiens
ctacctgcacgaacag

SEQ ID NO: 904 - DNA, 17 bp, Homo sapiens
tgctcaataaatacccg

SEQ ID NO: 905 - DNA, 18 bp, Homo sapiens
tgctcaataaatacccgt

SEQ ID NO: 906 - DNA, 19 bp, Homo sapiens
tgctcaataaatacccgtt

SEQ ID NO: 907 - DNA, 20 bp, Homo sapiens
tgctcaataaatacccgttg

SEQ ID NO: 908 - DNA, 18 bp, Homo sapiens
gcaaaaatgtgctagtgc

SEQ ID NO: 909 - DNA, 19 bp, Homo sapiens
gcaaaaatgtgctagtgcc

SEQ ID NO: 910 - DNA, 20 bp, Homo sapiens
gcaaaaatgtgctagtgcca

SEQ ID NO: 911 - DNA, 21 bp, Homo sapiens
aacaatacaacttactacctc

SEQ ID NO: 912 - DNA, 18 bp, Homo sapiens
cacaagatcggatctacg

SEQ ID NO: 913 - DNA, 14 bp, Homo sapiens
cgcaaggtcggttc

SEQ ID NO: 914 - DNA, 15 bp, Homo sapiens
cgcaaggtcggttct

SEQ ID NO: 915 - DNA, 16 bp, Homo sapiens
cgcaaggtcggttcta

SEQ ID NO: 916 - DNA, 21 bp, Homo sapiens
aactatacaacctactacctc

SEQ ID NO: 917 - DNA, 22 bp, Homo sapiens
aactatacaacctactacctca

SEQ ID NO: 918 - DNA, 16 bp, Homo sapiens
aaccacacaacctact

SEQ ID NO: 919 - DNA, 19 bp, Homo sapiens
aaccacacaacctactacc

SEQ ID NO: 920 - DNA, 21 bp, Homo sapiens
aaccatacaacctactacctc

SEQ ID NO: 921 - DNA, 18 bp, Homo sapiens
actatgcaacctactacc

SEQ ID NO: 922 - DNA, 20 bp, Homo sapiens
actatgcaacctactacctc

SEQ ID NO: 923 - DNA, 21 bp, Homo sapiens
actatgcaacctactacctct

SEQ ID NO: 924 - DNA, 18 bp, Homo sapiens
actatacaacctcctacc

SEQ ID NO: 925 - DNA, 19 bp, Homo sapiens
actatacaacctcctacct

SEQ ID NO: 926 - DNA, 17 bp, Homo sapiens
cacaaaccattatgtgc

SEQ ID NO: 927 - DNA, 18 bp, Homo sapiens
cacaaaccattatgtgct

SEQ ID NO: 928 - DNA, 16 bp, Homo sapiens
cgccaatatttacgtg

SEQ ID NO: 929 - DNA, 15 bp, Homo sapiens
acaagtgccttcact

SEQ ID NO: 930 - DNA, 16 bp, Homo sapiens
acaagtgccttcactg

SEQ ID NO: 931 - DNA, 16 bp, Homo sapiens
actacctgcactgtaa

SEQ ID NO: 932 - DNA, 17 bp, Homo sapiens
actacctgcactgtaag

SEQ ID NO: 933 - DNA, 18 bp, Homo sapiens
tcagttttgcatagattt

SEQ ID NO: 934 - DNA, 19 bp, Homo sapiens
tcagttttgcatagatttg

SEQ ID NO: 935 - DNA, 16 bp, Homo sapiens
tcagttttgcatggat

SEQ ID NO: 936 - DNA, 17 bp, Homo sapiens
tcagttttgcatggatt

SEQ ID NO: 937 - DNA, 18 bp, Homo sapiens
tcagttttgcatggattt

SEQ ID NO: 938 - DNA, 18 bp, Homo sapiens
tcaacatcagtctgataa

SEQ ID NO: 939 - DNA, 22 bp, Homo sapiens
tcaacatcagtctgataagcta

SEQ ID NO: 940 - DNA, 16 bp, Homo sapiens
acagttcttcaactgg

SEQ ID NO: 941 - DNA, 14 bp, Homo sapiens
ggaaatccctggca

SEQ ID NO: 942 - DNA, 15 bp, Homo sapiens
ggaaatccctggcaa

SEQ ID NO: 943 - DNA, 16 bp, Homo sapiens
ggaaatccctggcaat

SEQ ID NO: 944 - DNA, 17 bp, Homo sapiens
actgatatcagctcagt

SEQ ID NO: 945 - DNA, 18 bp, Homo sapiens
actgatatcagctcagta

SEQ ID NO: 946 - DNA, 19 bp, Homo sapiens
actgatatcagctcagtag

SEQ ID NO: 947 - DNA, 14 bp, Homo sapiens
ctgttcctgctgaa

SEQ ID NO: 948 - DNA, 15 bp, Homo sapiens
ctgttcctgctgaac

SEQ ID NO: 949 - DNA, 16 bp, Homo sapiens
ctgttcctgctgaact

SEQ ID NO: 950 - DNA, 15 bp, Homo sapiens
tcagaccgagacaag

SEQ ID NO: 951 - DNA, 16 bp, Homo sapiens
tcagaccgagacaagt

SEQ ID NO: 952 - DNA, 19 bp, Homo sapiens
gcctatcctggattacttg

SEQ ID NO: 953 - DNA, 14 bp, Homo sapiens
gcggaacttagcca

SEQ ID NO: 954 - DNA, 16 bp, Homo sapiens
aaccgatttcagatgg

SEQ ID NO: 955 - DNA, 17 bp, Homo sapiens
aaccgatttcagatggt

SEQ ID NO: 956 - DNA, 14 bp, Homo sapiens
gctgcaaacatccg

SEQ ID NO: 957 - DNA, 14 bp, Homo sapiens
cttccagtcgagga

SEQ ID NO: 958 - DNA, 17 bp, Homo sapiens
gcaacttagtaatgtgc

SEQ ID NO: 959 - DNA, 18 bp, Homo sapiens
gcaacttagtaatgtgca

SEQ ID NO: 960 - DNA, 17 bp, Homo sapiens
caatgcaactacaatgc

SEQ ID NO: 961 - DNA, 18 bp, Homo sapiens
caatgcaactacaatgca

SEQ ID NO: 962 - DNA, 14 bp, Homo sapiens
ctacctgcacgaac

SEQ ID NO: 963 - DNA, 15 bp, Homo sapiens
ctacctgcacgaaca

SEQ ID NO: 964 - DNA, 16 bp, Homo sapiens
gcaaaaatgtgctagt

SEQ ID NO: 965 - DNA, 17 bp, Homo sapiens
gcaaaaatgtgctagtg

SEQ ID NO: 966 - DNA, 19 bp, Homo sapiens
aacaatacaacttactacc

SEQ ID NO: 967 - DNA, 20 bp, Homo sapiens
aacaatacaacttactacct

SEQ ID NO: 968 - DNA, 22 bp, Homo sapiens
aacaatacaacttactacctca

SEQ ID NO: 969 - DNA, 15 bp, Homo sapiens
cacaagatcggatct

SEQ ID NO: 970 - DNA, 16 bp, Homo sapiens
cacaagatcggatcta

SEQ ID NO: 971 - DNA, 17 bp, Homo sapiens
cacaagatcggatctac

SEQ ID NO: 972 - DNA, 15 bp, Homo sapiens
cacaagttcggatct

SEQ ID NO: 973 - DNA, 16 bp, Homo sapiens
cacaagttcggatcta

SEQ ID NO: 974 - DNA, 18 bp, Homo sapiens
cttcagttatcacagtac

SEQ ID NO: 975 - DNA, 19 bp, Homo sapiens
cttcagttatcacagtact

SEQ ID NO: 976 - DNA, 20 bp, Homo sapiens
cttcagttatcacagtactg

SEQ ID NO: 977 - DNA, 21 bp, Homo sapiens
cttcagttatcacagtactgt

SEQ ID NO: 978 - DNA, 18 bp, Homo sapiens
aacactgatttcaaatgg

SEQ ID NO: 979 - DNA, 19 bp, Homo sapiens
aacactgatttcaaatggt

SEQ ID NO: 980 - DNA, 16 bp, Homo sapiens
tcatagccctgtacaa

SEQ ID NO: 981 - DNA, 19 bp, Homo sapiens
tcatagccctgtacaatgc

SEQ ID NO: 982 - DNA, 14 bp, Homo sapiens
gctacctgcactgt

SEQ ID NO: 983 - DNA, 15 bp, Homo sapiens
gctacctgcactgta

SEQ ID NO: 984 - DNA, 16 bp, Homo sapiens
gctacctgcactgtaa

SEQ ID NO: 985 - DNA, 16 bp, Homo sapiens
tgatagccctgtacaa

SEQ ID NO: 986 - DNA, 17 bp, Homo sapiens
tgatagccctgtacaat

SEQ ID NO: 987 - DNA, 18 bp, Homo sapiens
tgatagccctgtacaatg

SEQ ID NO: 988 - DNA, 16 bp, Homo sapiens
gaacaggtagtctgaa

SEQ ID NO: 989 - DNA, 17 bp, Homo sapiens
gaacaggtagtctgaac

SEQ ID NO: 990 - DNA, 18 bp, Homo sapiens
gaacaggtagtctgaaca

SEQ ID NO: 991 - DNA, 15 bp, Homo sapiens
aaccaatgtgcagac

SEQ ID NO: 992 - DNA, 16 bp, Homo sapiens
aaccaatgtgcagact

SEQ ID NO: 993 - DNA, 17 bp, Homo sapiens
aaccaatgtgcagacta

SEQ ID NO: 994 - DNA, 17 bp, Homo sapiens
acaagctttttgctcgt

SEQ ID NO: 995 - DNA, 17 bp, Homo sapiens
acaaagttctgtagtgc

SEQ ID NO: 996 - DNA, 18 bp, Homo sapiens
acaaagttctgtagtgca

SEQ ID NO: 997 - DNA, 15 bp, Homo sapiens
gctgagagtgtagga

SEQ ID NO: 998 - DNA, 16 bp, Homo sapiens
gctgagagtgtaggat

SEQ ID NO: 999 - DNA, 19 bp, Homo sapiens
gctgagagtgtaggatgtt

SEQ ID NO: 1000 - DNA, 16 bp, Homo sapiens
cttccagtcggggatg

SEQ ID NO: 1001 - DNA, 14 bp, Homo sapiens
agacacgtgcactg

SEQ ID NO: 1002 - DNA, 15 bp, Homo sapiens
agacacgtgcactgt

SEQ ID NO: 1003 - DNA, 16 bp, Homo sapiens
agacacgtgcactgta

SEQ ID NO: 1004 - DNA, 15 bp, Homo sapiens
gcagaagcatttcca

SEQ ID NO: 1005 - DNA, 17 bp, Homo sapiens
caacaaaatcactagtc

SEQ ID NO: 1006 - DNA, 18 bp, Homo sapiens
caacaaaatcactagtct

SEQ ID NO: 1007 - DNA, 19 bp, Homo sapiens
caacaaaatcactagtctt

SEQ ID NO: 1008 - DNA, 20 bp, Homo sapiens
caacaaaatcactagtcttc

SEQ ID NO: 1009 - DNA, 16 bp, Homo sapiens
cacaaattcggatcta

SEQ ID NO: 1010 - DNA, 17 bp, Homo sapiens
cacaaattcggatctac

SEQ ID NO: 1011 - DNA, 18 bp, Homo sapiens
cacaaattcggatctaca

SEQ ID NO: 1012 - DNA, 17 bp, Homo sapiens
acaaattcggttctaca

SEQ ID NO: 1013 - DNA, 18 bp, Homo sapiens
acaaattcggttctacag

SEQ ID NO: 1014 - DNA, 16 bp, Homo sapiens
aacaaccagctaagac

SEQ ID NO: 1015 - DNA, 17 bp, Homo sapiens
aacaaccagctaagaca

SEQ ID NO: 1016 - DNA, 18 bp, Homo sapiens
aacaaccagctaagacac

SEQ ID NO: 1017 - DNA, 14 bp, Homo sapiens
actcaccgacaggt

SEQ ID NO: 1018 - DNA, 15 bp, Homo sapiens
actcaccgacaggtt

SEQ ID NO: 1019 - DNA, 16 bp, Homo sapiens
actcaccgacaggttg

SEQ ID NO: 1020 - DNA, 19 bp, Homo sapiens
tgtgagttctaccattgcc

SEQ ID NO: 1021 - DNA, 18 bp, Homo sapiens
gaacagatagtctaaaca

SEQ ID NO: 1022 - DNA, 19 bp, Homo sapiens
gaacagatagtctaaacac

SEQ ID NO: 1023 - DNA, 20 bp, Homo sapiens
gaacagatagtctaaacact

SEQ ID NO: 1024 - DNA, 16 bp, Homo sapiens
aggcataggatgacaa

SEQ ID NO: 1025 - DNA, 17 bp, Homo sapiens
aggcataggatgacaaa

SEQ ID NO: 1026 - DNA, 18 bp, Homo sapiens
aggcataggatgacaaag

SEQ ID NO: 1027 - DNA, 15 bp, Homo sapiens
tcagccgctgtcaca

SEQ ID NO: 1028 - DNA, 14 bp, Homo sapiens
aggcgaaggatgac

SEQ ID NO: 1029 - DNA, 15 bp, Homo sapiens
aggcgaaggatgaca

SEQ ID NO: 1030 - DNA, 16 bp, Homo sapiens
aggcgaaggatgacaa

SEQ ID NO: 1031 - DNA, 17 bp, Homo sapiens
aggcgaaggatgacaaa

SEQ ID NO: 1032 - DNA, 15 bp, Homo sapiens
ctgcctgtctgtgcc

SEQ ID NO: 1033 - DNA, 17 bp, Homo sapiens
acatggttagatcaagc

SEQ ID NO: 1034 - DNA, 18 bp, Homo sapiens
acatggttagatcaagca

SEQ ID NO: 1035 - DNA, 14 bp, Homo sapiens
gaaacccagcagac

SEQ ID NO: 1036 - DNA, 15 bp, Homo sapiens
gaaacccagcagaca

SEQ ID NO: 1037 - DNA, 16 bp, Homo sapiens
gaaacccagcagacaa

SEQ ID NO: 1038 - DNA, 17 bp, Homo sapiens
gaaacccagcagacaat

SEQ ID NO: 1039 - DNA, 14 bp, Homo sapiens
gagacccagtagcc

SEQ ID NO: 1040 - DNA, 15 bp, Homo sapiens
gagacccagtagcca

SEQ ID NO: 1041 - DNA, 16 bp, Homo sapiens
ggggtatttgacaaac

SEQ ID NO: 1042 - DNA, 17 bp, Homo sapiens
ggggtatttgacaaact

SEQ ID NO: 1043 - DNA, 18 bp, Homo sapiens
ggggtatttgacaaactg

SEQ ID NO: 1044 - DNA, 16 bp, Homo sapiens
taaacggaaccactag

SEQ ID NO: 1045 - DNA, 17 bp, Homo sapiens
taaacggaaccactagt

SEQ ID NO: 1046 - DNA, 18 bp, Homo sapiens
taaacggaaccactagtg

SEQ ID NO: 1047 - DNA, 18 bp, Homo sapiens
actgtacaaactactacc

SEQ ID NO: 1048 - DNA, 17 bp, Homo sapiens
acagcacaaactactac

SEQ ID NO: 1049 - DNA, 14 bp, Homo sapiens
ggtaatccctggca

SEQ ID NO: 1050 - DNA, 15 bp, Homo sapiens
ggtaatccctggcaa

SEQ ID NO: 1051 - DNA, 16 bp, Homo sapiens
ggtaatccctggcaat

SEQ ID NO: 1052 - DNA, 15 bp, Homo sapiens
gcagaacttagccac

SEQ ID NO: 1053 - DNA, 16 bp, Homo sapiens
gcagaacttagccact

SEQ ID NO: 1054 - DNA, 21 bp, Homo sapiens
agctgagtgtaggatgtttac

SEQ ID NO: 1055 - DNA, 16 bp, Homo sapiens
tcacaagttagggtct

SEQ ID NO: 1056 - DNA, 19 bp, Homo sapiens
tcacaagttagggtctcag

SEQ ID NO: 1057 - DNA, 17 bp, Homo sapiens
atgcccttttaacattg

SEQ ID NO: 1058 - DNA, 20 bp, Homo sapiens
atgcccttttaacattgcac

SEQ ID NO: 1059 - DNA, 14 bp, Homo sapiens
cgaccatggctgta

SEQ ID NO: 1060 - DNA, 15 bp, Homo sapiens
acagctggttgaagg

SEQ ID NO: 1061 - DNA, 19 bp, Homo sapiens
tcacataggaataaaaagc

SEQ ID NO: 1062 - DNA, 20 bp, Homo sapiens
tcacataggaataaaaagcc

SEQ ID NO: 1063 - DNA, 17 bp, Homo sapiens
ctaccatagggtaaaac

SEQ ID NO: 1064 - DNA, 18 bp, Homo sapiens
ctaccatagggtaaaacc

SEQ ID NO: 1065 - DNA, 18 bp, Homo sapiens
tccataaagtaggaaaca

SEQ ID NO: 1066 - DNA, 19 bp, Homo sapiens
tccataaagtaggaaacac

SEQ ID NO: 1067 - DNA, 20 bp, Homo sapiens
tccataaagtaggaaacact

SEQ ID NO: 1068 - DNA, 21 bp, Homo sapiens
tccataaagtaggaaacacta

SEQ ID NO: 1069 - DNA, 17 bp, Homo sapiens
gtagtgctttctacttt

SEQ ID NO: 1070 - DNA, 18 bp, Homo sapiens
gtagtgctttctacttta

SEQ ID NO: 1071 - DNA, 16 bp, Homo sapiens
tgagctacagtgcttc

SEQ ID NO: 1072 - DNA, 17 bp, Homo sapiens
tgagctacagtgcttca

SEQ ID NO: 1073 - DNA, 18 bp, Homo sapiens
tgagctacagtgcttcat

SEQ ID NO: 1074 - DNA, 20 bp, Homo sapiens
ctagtacatcatctatactg

SEQ ID NO: 1075 - DNA, 21 bp, Homo sapiens
ctagtacatcatctatactgt

SEQ ID NO: 1076 - DNA, 15 bp, Homo sapiens
aagggattcctggga

SEQ ID NO: 1077 - DNA, 16 bp, Homo sapiens
aagggattcctgggaa

SEQ ID NO: 1078 - DNA, 17 bp, Homo sapiens
aagggattcctgggaaa

SEQ ID NO: 1079 - DNA, 14 bp, Homo sapiens
cccaagttctgtca

SEQ ID NO: 1080 - DNA, 15 bp, Homo sapiens
cccaagttctgtcat

SEQ ID NO: 1081 - DNA, 16 bp, Homo sapiens
cccaagttctgtcatg

SEQ ID NO: 1082 - DNA, 17 bp, Homo sapiens
agctgcttttgggattc

SEQ ID NO: 1083 - DNA, 18 bp, Homo sapiens
agctgcttttgggattcc

SEQ ID NO: 1084 - DNA, 18 bp, Homo sapiens
tcatacagctagataacc

SEQ ID NO: 1085 - DNA, 15 bp, Homo sapiens
cacaggttaaagggt

SEQ ID NO: 1086 - DNA, 16 bp, Homo sapiens
cacaggttaaagggtc

SEQ ID NO: 1087 - DNA, 17 bp, Homo sapiens
cacaggttaaagggtct

SEQ ID NO: 1088 - DNA, 16 bp, Homo sapiens
gcattattactcacgg

SEQ ID NO: 1089 - DNA, 17 bp, Homo sapiens
gcattattactcacggt

SEQ ID NO: 1090 - DNA, 18 bp, Homo sapiens
gcattattactcacggta

SEQ ID NO: 1091 - DNA, 14 bp, Homo sapiens
cgcgtaccaaaagt

SEQ ID NO: 1092 - DNA, 15 bp, Homo sapiens
cgcgtaccaaaagta

SEQ ID NO: 1093 - DNA, 16 bp, Homo sapiens
cgcgtaccaaaagtaa

SEQ ID NO: 1094 - DNA, 17 bp, Homo sapiens
cgcgtaccaaaagtaat

SEQ ID NO: 1095 - DNA, 18 bp, Homo sapiens
cgcgtaccaaaagtaata

SEQ ID NO: 1096 - DNA, 19 bp, Homo sapiens
cgcgtaccaaaagtaataa

SEQ ID NO: 1097 - DNA, 20 bp, Homo sapiens
cgcgtaccaaaagtaataat

SEQ ID NO: 1098 - DNA, 14 bp, Homo sapiens
agccaagctcagac

SEQ ID NO: 1099 - DNA, 15 bp, Homo sapiens
agccaagctcagacg

SEQ ID NO: 1100 - DNA, 16 bp, Homo sapiens
ccctctggtcaaccag

SEQ ID NO: 1101 - DNA, 17 bp, Homo sapiens
tccatcatcaaaacaaa

SEQ ID NO: 1102 - DNA, 18 bp, Homo sapiens
tccatcatcaaaacaaat

SEQ ID NO: 1103 - DNA, 19 bp, Homo sapiens
tccatcatcaaaacaaatg

SEQ ID NO: 1104 - DNA, 20 bp, Homo sapiens
tccatcatcaaaacaaatgg

SEQ ID NO: 1105 - DNA, 17 bp, Homo sapiens
acccttatcagttctcc

SEQ ID NO: 1106 - DNA, 16 bp, Homo sapiens
gaactgcctttctctc

SEQ ID NO: 1107 - DNA, 17 bp, Homo sapiens
gaactgcctttctctcc

SEQ ID NO: 1108 - DNA, 15 bp, Homo sapiens
aagcccaaaaggaga

SEQ ID NO: 1109 - DNA, 16 bp, Homo sapiens
aagcccaaaaggagaa

SEQ ID NO: 1110 - DNA, 17 bp, Homo sapiens
aagcccaaaaggagaat

SEQ ID NO: 1111 - DNA, 18 bp, Homo sapiens
aagcccaaaaggagaatt

SEQ ID NO: 1112 - DNA, 16 bp, Homo sapiens
accctccaccatgcaa

SEQ ID NO: 1113 - DNA, 17 bp, Homo sapiens
tccacatggagttgctg

SEQ ID NO: 1114 - DNA, 16 bp, Homo sapiens
gccaatatttctgtgc

SEQ ID NO: 1115 - DNA, 17 bp, Homo sapiens
gccaatatttctgtgct

SEQ ID NO: 1116 - DNA, 14 bp, Homo sapiens
gaaagagaccggtt

SEQ ID NO: 1117 - DNA, 15 bp, Homo sapiens
gaaagagaccggttc

SEQ ID NO: 1118 - DNA, 16 bp, Homo sapiens
gaaagagaccggttca

SEQ ID NO: 1119 - DNA, 15 bp, Homo sapiens
atctgcactgtcagc

SEQ ID NO: 1120 - DNA, 17 bp, Homo sapiens
atctgcactgtcagcac

SEQ ID NO: 1121 - DNA, 18 bp, Homo sapiens
atctgcactgtcagcact

SEQ ID NO: 1122 - DNA, 16 bp, Homo sapiens
accgatttcaaatggt

SEQ ID NO: 1123 - DNA, 17 bp, Homo sapiens
accgatttcaaatggtg

SEQ ID NO: 1124 - DNA, 16 bp, Homo sapiens
acatcgttaccagaca

SEQ ID NO: 1125 - DNA, 19 bp, Homo sapiens
acatcgttaccagacagtg

SEQ ID NO: 1126 - DNA, 16 bp, Homo sapiens
tcaccaaaacatggaa

SEQ ID NO: 1127 - DNA, 17 bp, Homo sapiens
tcaccaaaacatggaag

SEQ ID NO: 1128 - DNA, 17 bp, Homo sapiens
gcaatcagctaactaca

SEQ ID NO: 1129 - DNA, 20 bp, Homo sapiens
gcaatcagctaactacactg

SEQ ID NO: 1130 - DNA, 18 bp, Homo sapiens
gctttgacaatactattg

SEQ ID NO: 1131 - DNA, 21 bp, Homo sapiens
gctttgacaatactattgcac

SEQ ID NO: 1132 - DNA, 16 bp, Homo sapiens
acaggattgagggggg

SEQ ID NO: 1133 - DNA, 18 bp, Homo sapiens
atgccctttcatcattgc

SEQ ID NO: 1134 - DNA, 19 bp, Homo sapiens
atgccctttcatcattgca

SEQ ID NO: 1135 - DNA, 15 bp, Homo sapiens
gctgtaaacatccga

SEQ ID NO: 1136 - DNA, 15 bp, Homo sapiens
tccagtcaaggatgt

SEQ ID NO: 1137 - DNA, 16 bp, Homo sapiens
tccagtcaaggatgtt

SEQ ID NO: 1138 - DNA, 17 bp, Homo sapiens
tccagtcaaggatgttt

SEQ ID NO: 1139 - DNA, 18 bp, Homo sapiens
tccagtcaaggatgttta

SEQ ID NO: 1140 - DNA, 19 bp, Homo sapiens
tccagtcaaggatgtttac

SEQ ID NO: 1141 - DNA, 16 bp, Homo sapiens
gtacccctggagattc

SEQ ID NO: 1142 - DNA, 17 bp, Homo sapiens
gtacccctggagattct

SEQ ID NO: 1143 - DNA, 18 bp, Homo sapiens
ctactaaaacatggaagc

SEQ ID NO: 1144 - DNA, 19 bp, Homo sapiens
ctactaaaacatggaagca

SEQ ID NO: 1145 - DNA, 15 bp, Homo sapiens
cagcaggtaccccca

SEQ ID NO: 1146 - DNA, 16 bp, Homo sapiens
acactcaaacatggaa

SEQ ID NO: 1147 - DNA, 17 bp, Homo sapiens
acactcaaacatggaag

SEQ ID NO: 1148 - DNA, 18 bp, Homo sapiens
acactcaaacatggaagc

SEQ ID NO: 1149 - DNA, 17 bp, Homo sapiens
tcaccattgctaaagtg

SEQ ID NO: 1150 - DNA, 16 bp, Homo sapiens
aaacgtggaatttcct

SEQ ID NO: 1151 - DNA, 17 bp, Homo sapiens
aaacgtggaatttcctc

SEQ ID NO: 1152 - DNA, 18 bp, Homo sapiens
aaacgtggaatttcctct

SEQ ID NO: 1153 - DNA, 19 bp, Homo sapiens
aaacgtggaatttcctcta

SEQ ID NO: 1154 - DNA, 20 bp, Homo sapiens
aaacgtggaatttcctctat

SEQ ID NO: 1155 - DNA, 15 bp, Homo sapiens
acaccccaaaatcga

SEQ ID NO: 1156 - DNA, 14 bp, Homo sapiens
ggaaagcgccccca

SEQ ID NO: 1157 - DNA, 18 bp, Homo sapiens
cacttatcaggttgtatt

SEQ ID NO: 1158 - DNA, 19 bp, Homo sapiens
cacttatcaggttgtatta

SEQ ID NO: 1159 - DNA, 20 bp, Homo sapiens
cacttatcaggttgtattat

SEQ ID NO: 1160 - DNA, 21 bp, Homo sapiens
cacttatcaggttgtattata

SEQ ID NO: 1161 - DNA, 15 bp, Homo sapiens
acgtggattttcctc

SEQ ID NO: 1162 - DNA, 16 bp, Homo sapiens
acgtggattttcctct

SEQ ID NO: 1163 - DNA, 17 bp, Homo sapiens
acgtggattttcctcta

SEQ ID NO: 1164 - DNA, 18 bp, Homo sapiens
acgtggattttcctctat

SEQ ID NO: 1165 - DNA, 16 bp, Homo sapiens
acaaaagttgcctttg

SEQ ID NO: 1166 - DNA, 17 bp, Homo sapiens
acaaaagttgcctttgt

SEQ ID NO: 1167 - DNA, 14 bp, Homo sapiens
acacaggacctgga

SEQ ID NO: 1168 - DNA, 15 bp, Homo sapiens
acacaggacctggag

SEQ ID NO: 1169 - DNA, 16 bp, Homo sapiens
acacaggacctggagt

SEQ ID NO: 1170 - DNA, 14 bp, Homo sapiens
ggccttctgactcc

SEQ ID NO: 1171 - DNA, 17 bp, Homo sapiens
tacgttccatagtctac

SEQ ID NO: 1172 - DNA, 15 bp, Homo sapiens
agccacaatcacctt

SEQ ID NO: 1173 - DNA, 16 bp, Homo sapiens
agccacaatcaccttc

SEQ ID NO: 1174 - DNA, 17 bp, Homo sapiens
agccacaatcaccttct

SEQ ID NO: 1175 - DNA, 18 bp, Homo sapiens
ggctataaagtaactgag

SEQ ID NO: 1176 - DNA, 19 bp, Homo sapiens
ggctataaagtaactgaga

SEQ ID NO: 1177 - DNA, 14 bp, Homo sapiens
acggaagggcagag

SEQ ID NO: 1178 - DNA, 15 bp, Homo sapiens
acggaagggcagaga

SEQ ID NO: 1179 - DNA, 14 bp, Homo sapiens
gacgggtgcgattt

SEQ ID NO: 1180 - DNA, 14 bp, Homo sapiens
cctcaaggagcttc

SEQ ID NO: 1181 - DNA, 15 bp, Homo sapiens
cctcaaggagcttca

SEQ ID NO: 1182 - DNA, 17 bp, Homo sapiens
acaaagttctgtgatgc

SEQ ID NO: 1183 - DNA, 18 bp, Homo sapiens
acaaagttctgtgatgca

SEQ ID NO: 1184 - DNA, 16 bp, Homo sapiens
ttctaggataggccca

SEQ ID NO: 1185 - DNA, 17 bp, Homo sapiens
ttctaggataggcccag

SEQ ID NO: 1186 - DNA, 15 bp, Homo sapiens
acaccaatgccctag

SEQ ID NO: 1187 - DNA, 18 bp, Homo sapiens
tcaacaaaatcactgatg

SEQ ID NO: 1188 - DNA, 19 bp, Homo sapiens
tcaacaaaatcactgatgc

SEQ ID NO: 1189 - DNA, 20 bp, Homo sapiens
tcaacaaaatcactgatgct

SEQ ID NO: 1190 - DNA, 14 bp, Homo sapiens
tgagctcctggagg

SEQ ID NO: 1191 - DNA, 15 bp, Homo sapiens
tgagctcctggagga

SEQ ID NO: 1192 - DNA, 18 bp, Homo sapiens
acatttttcgttattgct

SEQ ID NO: 1193 - DNA, 15 bp, Homo sapiens
tagctggttgaaggg

SEQ ID NO: 1194 - DNA, 16 bp, Homo sapiens
tagctggttgaagggg

SEQ ID NO: 1195 - DNA, 14 bp, Homo sapiens
gccctggactagga

SEQ ID NO: 1196 - DNA, 14 bp, Homo sapiens
ggccttctgaccct

SEQ ID NO: 1197 - DNA, 15 bp, Homo sapiens
ggccttctgacccta

SEQ ID NO: 1198 - DNA, 16 bp, Homo sapiens
ggccttctgaccctaa

SEQ ID NO: 1199 - DNA, 15 bp, Homo sapiens
ctgaggggcctcaga

SEQ ID NO: 1200 - DNA, 17 bp, Homo sapiens
ttcaaaacatgaattgc

SEQ ID NO: 1201 - DNA, 18 bp, Homo sapiens
ttcaaaacatgaattgct

SEQ ID NO: 1202 - DNA, 19 bp, Homo sapiens
ttcaaaacatgaattgctg

SEQ ID NO: 1203 - DNA, 20 bp, Homo sapiens
ttcaaaacatgaattgctgc

SEQ ID NO: 1204 - DNA, 15 bp, Drosophila melanogaster
tgagacacactttgc

SEQ ID NO: 1205 - DNA, 16 bp, Drosophila melanogaster
tgagacacactttgcc

SEQ ID NO: 1206 - DNA, 19 bp, Drosophila melanogaster
actatacaacctactacct

SEQ ID NO: 1207 - DNA, 20 bp, Drosophila melanogaster
actatacaacctactacctc

SEQ ID NO: 1208 - DNA, 21 bp, Drosophila melanogaster
actatacaacctactacctca

SEQ ID NO: 1209 - DNA, 18 bp, Drosophila melanogaster
taggagagagaaaaagac

SEQ ID NO: 1210 - DNA, 19 bp, Drosophila melanogaster
taggagagagaaaaagact

SEQ ID NO: 1211 - DNA, 20 bp, Drosophila melanogaster
taggagagagaaaaagactg

SEQ ID NO: 1212 - DNA, 21 bp, Drosophila melanogaster
taggagagagaaaaagactga

SEQ ID NO: 1213 - DNA, 14 bp, Drosophila melanogaster
tcagctatgccgac

SEQ ID NO: 1214 - DNA, 15 bp, Drosophila melanogaster
tcagctatgccgaca

SEQ ID NO: 1215 - DNA, 16 bp, Drosophila melanogaster
tcagctatgccgacat

SEQ ID NO: 1216 - DNA, 17 bp, Drosophila melanogaster
tcagctatgccgacatc

SEQ ID NO: 1217 - DNA, 14 bp, Drosophila melanogaster
gctcctcaaagctg

SEQ ID NO: 1218 - DNA, 16 bp, Drosophila melanogaster
aaaaagaacagccact

SEQ ID NO: 1219 - DNA, 17 bp, Drosophila melanogaster
aaaaagaacagccactg

SEQ ID NO: 1220 - DNA, 18 bp, Drosophila melanogaster
aaaaagaacagccactgt

SEQ ID NO: 1221 - DNA, 14 bp, Drosophila melanogaster
cggggcgagagaat

SEQ ID NO: 1222 - DNA, 15 bp, Drosophila melanogaster
cggggcgagagaatg

SEQ ID NO: 1223 - DNA, 16 bp, Drosophila melanogaster
gcactgatttcgaatg

SEQ ID NO: 1224 - DNA, 17 bp, Drosophila melanogaster
gcactgatttcgaatgg

SEQ ID NO: 1225 - DNA, 18 bp, Drosophila melanogaster
ctcacagtataatcctgt

SEQ ID NO: 1226 - DNA, 19 bp, Drosophila melanogaster
ctcacagtataatcctgtg

SEQ ID NO: 1227 - DNA, 20 bp, Drosophila melanogaster
ctcacagtataatcctgtga

SEQ ID NO: 1228 - DNA, 21 bp, Drosophila melanogaster
ctcacagtataatcctgtgat

SEQ ID NO: 1229 - DNA, 14 bp, Drosophila melanogaster
cgccagtaagcgga

SEQ ID NO: 1230 - DNA, 15 bp, Drosophila melanogaster
cgccagtaagcggaa

SEQ ID NO: 1231 - DNA, 15 bp, Drosophila melanogaster
gctcatcaaagctgg

SEQ ID NO: 1232 - DNA, 16 bp, Drosophila melanogaster
gctcatcaaagctggc

SEQ ID NO: 1233 - DNA, 17 bp, Drosophila melanogaster
gctcatcaaagctggct

SEQ ID NO: 1234 - DNA, 14 bp, Drosophila melanogaster
gcccatcaaagctg

SEQ ID NO: 1235 - DNA, 15 bp, Drosophila melanogaster
gcccatcaaagctgg

SEQ ID NO: 1236 - DNA, 15 bp, Drosophila melanogaster
cagctattccgacat

SEQ ID NO: 1237 - DNA, 16 bp, Drosophila melanogaster
cagctattccgacatc

SEQ ID NO: 1238 - DNA, 17 bp, Drosophila melanogaster
cagctattccgacatct

SEQ ID NO: 1239 - DNA, 18 bp, Drosophila melanogaster
cagctattccgacatctt

SEQ ID NO: 1240 - DNA, 19 bp, Drosophila melanogaster
cagctattccgacatcttg

SEQ ID NO: 1241 - DNA, 19 bp, Homo sapiens
ctgcactataagcacttta

SEQ ID NO: 1242 - DNA, 20 bp, Homo sapiens
cctgcactataagcacttta

SEQ ID NO: 1243 - DNA, 21 bp, Homo sapiens
acctgcactataagcacttta

SEQ ID NO: 1244 - DNA, 22 bp, Homo sapiens
tacctgcactataagcacttta

SEQ ID NO: 1245 - DNA, 23 bp, Homo sapiens
ctacctgcactataagcacttta

SEQ ID NO: 1246 - DNA, 19 bp, Homo sapiens
ctgcactatgagcactttg

SEQ ID NO: 1247 - DNA, 20 bp, Homo sapiens
cctgcactatgagcactttg

SEQ ID NO: 1248 - DNA, 21 bp, Homo sapiens
acctgcactatgagcactttg

SEQ ID NO: 1249 - DNA, 22 bp, Homo sapiens
tacctgcactatgagcactttg

SEQ ID NO: 1250 - DNA, 23 bp, Homo sapiens
ctacctgcactatgagcactttg

SEQ ID NO: 1251 - DNA, 20 bp, Homo sapiens
cgctcgggttttcccgagcg

SEQ ID NO: 1252 - DNA, 30 bp, Homo sapiens
gaactatacaacctactacctttttttttt

SEQ ID NO: 1253 - DNA, 31 bp, Homo sapiens
gaactatacaacctactaccttttttttttt

SEQ ID NO: 1254 - DNA, 28 bp, Homo sapiens
gaaccacacaacctactatttttttttt

SEQ ID NO: 1255 - DNA, 29 bp, Homo sapiens
gaaccacacaacctactactttttttttt

SEQ ID NO: 1256 - DNA, 29 bp, Homo sapiens
gaaccatacaacctactactttttttttt

SEQ ID NO: 1257 - DNA, 30 bp, Homo sapiens
gaaccatacaacctactacctttttttttt

SEQ ID NO: 1258 - DNA, 31 bp, Homo sapiens
gaaccatacaacctactaccttttttttttt

SEQ ID NO: 1259 - DNA, 30 bp, Homo sapiens
gactatgcaacctactaccttttttttttt

SEQ ID NO: 1260 - DNA, 50 bp, Homo sapiens
cgctcgggttttcccgagcggaactatacaacctactacctttttttttt

SEQ ID NO: 1261 - DNA, 51 bp, Homo sapiens
cgctcgggttttcccgagcggaactatacaacctactaccttttttttttt

SEQ ID NO: 1262 - DNA, 48 bp, Homo sapiens
cgctcgggttttcccgagcggaaccacacaacctactatttttttttt

SEQ ID NO: 1263 - DNA, 49 bp, Homo sapiens
cgctcgggttttcccgagcggaaccacacaacctactactttttttttt

SEQ ID NO: 1264 - DNA, 49 bp, Homo sapiens
cgctcgggttttcccgagcggaaccatacaacctactactttttttttt

SEQ ID NO: 1265 - DNA, 50 bp, Homo sapiens
cgctcgggttttcccgagcggaaccatacaacctactacctttttttttt

SEQ ID NO: 1266 - DNA, 51 bp, Homo sapiens
cgctcgggttttcccgagcggaaccatacaacctactaccttttttttttt

SEQ ID NO: 1267 - DNA, 50 bp, Homo sapiens
cgctcgggttttcccgagcggactatgcaacctactaccttttttttttt

SEQ ID NO: 1268 - RNA, 22 nt, Homo sapiens
ugagguaguagguuguauaguu

SEQ ID NO: 1269 - RNA, 22 nt, Homo sapiens
ugagguaguagguugugugguu

SEQ ID NO: 1270 - RNA, 22 nt, Homo sapiens
ugagguaguagguuguaugguu

SEQ ID NO: 1271 - RNA, 21 nt, Homo sapiens
agagguaguagguugcauagu

SEQ ID NO: 1272 - RNA, 21 nt, Homo sapiens
ugagguaggagguuguauagu

SEQ ID NO: 1273 - RNA, 22 nt, Homo sapiens
ugagguaguagauuguauaguu

SEQ ID NO: 1274 - RNA, 21 nt, Homo sapiens
ugagguaguaguuuguacagu

SEQ ID NO: 1275 - RNA, 21 nt, Homo sapiens
ugagguaguaguuugugcugu

SEQ ID NO: 1276 - RNA, 21 nt, Homo sapiens
uggaauguaaagaaguaugua

SEQ ID NO: 1277 - RNA, 22 nt, Homo sapiens
aacccguagauccgaacuugug

SEQ ID NO: 1278 - RNA, 22 nt, Homo sapiens
uacaguacugugauaacugaag

SEQ ID NO: 1279 - RNA, 23 nt, Homo sapiens
agcagcauuguacagggcuauga

SEQ ID NO: 1280 - RNA, 20 nt, Homo sapiens
ucaaaugcucagacuccugu

SEQ ID NO: 1281 - RNA, 24 nt, Homo sapiens
aaaagugcuuacagugcagguagc

SEQ ID NO: 1282 - RNA, 21 nt, Homo sapiens
uaaagugcugacagugcagau

SEQ ID NO: 1283 - RNA, 23 nt, Homo sapiens
agcagcauuguacagggcuauca

SEQ ID NO: 1284 - RNA, 21 nt, Homo sapiens
auaaggauuuuuaggggcauu

SEQ ID NO: 1285 - RNA, 23 nt, Homo sapiens
uacccuguagauccgaauuugug

SEQ ID NO: 1286 - RNA, 22 nt, Homo sapiens
uacccuguagaaccgaauuugu

SEQ ID NO: 1287 - RNA, 23 nt, Homo sapiens
uggagugugacaaugguguuugu

SEQ ID NO: 1288 - RNA, 22 nt, Homo sapiens
uuaaggcacgcggugaaugcca

SEQ ID NO: 1289 - RNA, 23 nt, Homo sapiens
ucccugagacccuuuaaccugug

SEQ ID NO: 1290 - RNA, 22 nt, Homo sapiens
ucccugagacccuaacuuguga

SEQ ID NO: 1291 - RNA, 21 nt, Homo sapiens
ucguaccgugaguaauaaugc

SEQ ID NO: 1292 - RNA, 21 nt, Homo sapiens
cauuauuacuuuugguacgcg

SEQ ID NO: 1293 - RNA, 22 nt, Homo sapiens
ucggauccgucugagcuuggcu

SEQ ID NO: 1294 - RNA, 22 nt, Homo sapiens
ucacagugaaccggucucuuuu

SEQ ID NO: 1295 - RNA, 22 nt, Homo sapiens
ucacagugaaccggucucuuuc

SEQ ID NO: 1296 - RNA, 21 nt, Homo sapiens
cuuuuugcggucugggcuugc

SEQ ID NO: 1297 - RNA, 22 nt, Homo sapiens
cagugcaauguuaaaagggcau

SEQ ID NO: 1298 - RNA, 22 nt, Homo sapiens
cagugcaaugaugaaagggcau

SEQ ID NO: 1299 - RNA, 22 nt, Homo sapiens
uaacagucuacagccauggucg

SEQ ID NO: 1300 - RNA, 22 nt, Homo sapiens
uugguccccuucaaccagcugu

SEQ ID NO: 1301 - RNA, 21 nt, Homo sapiens
uugguccccuucaaccagcua

SEQ ID NO: 1302 - RNA, 21 nt, Homo sapiens
ugugacugguugaccagaggg

SEQ ID NO: 1303 - RNA, 23 nt, Homo sapiens
uauggcuuuuuauuccuauguga

SEQ ID NO: 1304 - RNA, 22 nt, Homo sapiens
uauggcuuuucauuccuaugug

SEQ ID NO: 1305 - RNA, 23 nt, Homo sapiens
acuccauuuguuuugaugaugga

SEQ ID NO: 1306 - RNA, 22 nt, Homo sapiens
uauugcuuaagaauacgcguag

SEQ ID NO: 1307 - RNA, 17 nt, Homo sapiens
agcugguguugugaauc

SEQ ID NO: 1308 - RNA, 18 nt, Homo sapiens
ucuacagugcacgugucu

SEQ ID NO: 1309 - RNA, 21 nt, Homo sapiens
agugguuuuacccuaugguag

SEQ ID NO: 1310 - RNA, 22 nt, Homo sapiens
uaacacugucugguaaagaugg

SEQ ID NO: 1311 - RNA, 23 nt, Homo sapiens
uguaguguuuccuacuuuaugga

SEQ ID NO: 1312 - RNA, 20 nt, Homo sapiens
cauaaaguagaaagcacuac

SEQ ID NO: 1313 - RNA, 22 nt, Homo sapiens
ugagaugaagcacuguagcuca

SEQ ID NO: 1314 - RNA, 22 nt, Homo sapiens
uacaguauagaugauguacuag

SEQ ID NO: 1315 - RNA, 24 nt, Homo sapiens
guccaguuuucccaggaaucccuu

SEQ ID NO: 1316 - RNA, 22 nt, Homo sapiens
ugagaacugaauuccauggguu

SEQ ID NO: 1317 - RNA, 22 nt, Homo sapiens
ugagaacugaauuccauaggcu

SEQ ID NO: 1318 - RNA, 20 nt, Homo sapiens
guguguggaaaugcuucugc

SEQ ID NO: 1319 - RNA, 22 nt, Homo sapiens
ucagugcacuacagaacuuugu

SEQ ID NO: 1320 - RNA, 22 nt, Homo sapiens
ucagugcaucacagaacuuugu

SEQ ID NO: 1321 - RNA, 22 nt, Homo sapiens
ucuggcuccgugucuucacucc

SEQ ID NO: 1322 - RNA, 22 nt, Homo sapiens
ucucccaacccuuguaccagug

SEQ ID NO: 1323 - RNA, 22 nt, Homo sapiens
acuagacugaagcuccuugagg

SEQ ID NO: 1324 - RNA, 22 nt, Homo sapiens
ucagugcaugacagaacuuggg

SEQ ID NO: 1325 - RNA, 20 nt, Homo sapiens
uugcauagucacaaaaguga

SEQ ID NO: 1326 - RNA, 22 nt, Homo sapiens
uagguuauccguguugccuucg

SEQ ID NO: 1327 - RNA, 22 nt, Homo sapiens
aaucauacacgguugaccuauu

SEQ ID NO: 1328 - RNA, 22 nt, Homo sapiens
uuaaugcuaaucgugauagggg

SEQ ID NO: 1329 - RNA, 22 nt, Homo sapiens
uagcagcacauaaugguuugug

SEQ ID NO: 1330 - RNA, 22 nt, Homo sapiens
uagcagcacaucaugguuuaca

SEQ ID NO: 1331 - RNA, 22 nt, Homo sapiens
uagcagcacguaaauauuggcg

SEQ ID NO: 1332 - RNA, 20 nt, Homo sapiens
acugcagugaaggcacuugu

SEQ ID NO: 1333 - RNA, 24 nt, Homo sapiens
caaagugcuuacagugcagguagu

SEQ ID NO: 1334 - RNA, 23 nt, Homo sapiens
aacauucaacgcugucggugagu

SEQ ID NO: 1335 - RNA, 22 nt, Homo sapiens
aacauucauugcugucgguggg

SEQ ID NO: 1336 - RNA, 22 nt, Homo sapiens
aacauucaaccugucggugagu

SEQ ID NO: 1337 - RNA, 24 nt, Homo sapiens
aacauucauuguugucgguggguu

SEQ ID NO: 1338 - RNA, 22 nt, Homo sapiens
uuuggcaaugguagaacucaca

SEQ ID NO: 1339 - RNA, 21 nt, Homo sapiens
ugguucuagacuugccaacua

SEQ ID NO: 1340 - RNA, 23 nt, Homo sapiens
uauggcacugguagaauucacug

SEQ ID NO: 1341 - RNA, 22 nt, Homo sapiens
uggacggagaacugauaagggu

SEQ ID NO: 1342 - RNA, 18 nt, Homo sapiens
uggagagaaaggcaguuc

SEQ ID NO: 1343 - RNA, 23 nt, Homo sapiens
caaagaauucuccuuuugggcuu

SEQ ID NO: 1344 - RNA, 21 nt, Homo sapiens
ucgugucuuguguugcagccg

SEQ ID NO: 1345 - RNA, 22 nt, Homo sapiens
caucccuugcaugguggagggu

SEQ ID NO: 1346 - RNA, 23 nt, Homo sapiens
gugccuacugagcugauaucagu

SEQ ID NO: 1347 - RNA, 22 nt, Homo sapiens
uaaggugcaucuagugcagaua

SEQ ID NO: 1348 - RNA, 22 nt, Homo sapiens
uaaggugcaucuagugcaguua

SEQ ID NO: 1349 - RNA, 22 nt, Homo sapiens
ugauauguuugauauauuaggu

SEQ ID NO: 1350 - RNA, 22 nt, Homo sapiens
caacggaaucccaaaagcagcu

SEQ ID NO: 1351 - RNA, 22 nt, Homo sapiens
gcugcgcuuggauuucgucccc

SEQ ID NO: 1352 - RNA, 21 nt, Homo sapiens
cugaccuaugaauugacagcc

SEQ ID NO: 1353 - RNA, 21 nt, Homo sapiens
aacuggccuacaaagucccag

SEQ ID NO: 1354 - RNA, 24 nt, Homo sapiens
aacuggcccucaaagucccgcuuu

SEQ ID NO: 1355 - RNA, 22 nt, Homo sapiens
uguaacagcaacuccaugugga

SEQ ID NO: 1356 - RNA, 21 nt, Homo sapiens
uagcagcacagaaauauuggc

SEQ ID NO: 1357 - RNA, 21 nt, Homo sapiens
uagguaguuucauguuguugg

SEQ ID NO: 1358 - RNA, 21 nt, Homo sapiens
uagguaguuuccuguuguugg

SEQ ID NO: 1359 - RNA, 22 nt, Homo sapiens
uucaccaccuucuccacccagc

SEQ ID NO: 1360 - RNA, 19 nt, Homo sapiens
gguccagaggggagauagg

SEQ ID NO: 1361 - RNA, 22 nt, Homo sapiens
uacaguagucugcacauugguu

SEQ ID NO: 1362 - RNA, 23 nt, Homo sapiens
cccaguguucagacuaccuguuc

SEQ ID NO: 1363 - RNA, 23 nt, Homo sapiens
cccaguguuuagacuaucuguuc

SEQ ID NO: 1364 - RNA, 23 nt, Homo sapiens
ugugcaaaucuaugcaaaacuga

SEQ ID NO: 1365 - RNA, 23 nt, Homo sapiens
ugugcaaauccaugcaaaacuga

SEQ ID NO: 1366 - RNA, 22 nt, Homo sapiens
caucuuaccggacagugcugga

SEQ ID NO: 1367 - RNA, 22 nt, Homo sapiens
uaacacugucugguaacgaugu

SEQ ID NO: 1368 - RNA, 23 nt, Homo sapiens
uaauacugccugguaaugaugac

SEQ ID NO: 1369 - RNA, 22 nt, Homo sapiens
uaauacugccggguaaugaugg

SEQ ID NO: 1370 - RNA, 22 nt, Homo sapiens
agagguauagggcaugggaaaa

SEQ ID NO: 1371 - RNA, 22 nt, Homo sapiens
uuuccuaugcauauacuucuuu

SEQ ID NO: 1372 - RNA, 22 nt, Homo sapiens
gugaaauguuuaggaccacuag

SEQ ID NO: 1373 - RNA, 22 nt, Homo sapiens
uucccuuugucauccuaugccu

SEQ ID NO: 1374 - RNA, 22 nt, Homo sapiens
uccuucauuccaccggagucug

SEQ ID NO: 1375 - RNA, 22 nt, Homo sapiens
uggaauguaaggaagugugugg

SEQ ID NO: 1376 - RNA, 22 nt, Homo sapiens
auaagacgagcaaaaagcuugu

SEQ ID NO: 1377 - RNA, 23 nt, Homo sapiens
uaaagugcuuauagugcagguag

SEQ ID NO: 1378 - RNA, 23 nt, Homo sapiens
caaagugcucauagugcagguag

SEQ ID NO: 1379 - RNA, 22 nt, Homo sapiens
uagcuuaucagacugauguuga

SEQ ID NO: 1380 - RNA, 22 nt, Homo sapiens
cugugcgugugacagcggcuga

SEQ ID NO: 1381 - RNA, 22 nt, Homo sapiens
uucccuuugucauccuucgccu

SEQ ID NO: 1382 - RNA, 21 nt, Homo sapiens
uaacagucuccagucacggcc

SEQ ID NO: 1383 - RNA, 22 nt, Homo sapiens
accaucgaccguugauuguacc

SEQ ID NO: 1384 - RNA, 21 nt, Homo sapiens
acagcaggcacagacaggcag

SEQ ID NO: 1385 - RNA, 21 nt, Homo sapiens
augaccuaugaauugacagac

SEQ ID NO: 1386 - RNA, 21 nt, Homo sapiens
uaaucucagcuggcaacugug

SEQ ID NO: 1387 - RNA, 24 nt, Homo sapiens
uacugcaucaggaacugauuggau

SEQ ID NO: 1388 - RNA, 21 nt, Homo sapiens
uugugcuugaucuaaccaugu

SEQ ID NO: 1389 - RNA, 21 nt, Homo sapiens
ugauuguccaaacgcaauucu

SEQ ID NO: 1390 - RNA, 22 nt, Homo sapiens
aagcugccaguugaagaacugu

SEQ ID NO: 1391 - RNA, 21 nt, Homo sapiens
ccacaccguaucugacacuuu

SEQ ID NO: 1392 - RNA, 23 nt, Homo sapiens
agcuacauugucugcuggguuuc

SEQ ID NO: 1393 - RNA, 24 nt, Homo sapiens
agcuacaucuggcuacugggucuc

SEQ ID NO: 1394 - RNA, 21 nt, Homo sapiens
ugucaguuugucaaauacccc

SEQ ID NO: 1395 - RNA, 23 nt, Homo sapiens
caagucacuagugguuccguuua

SEQ ID NO: 1396 - RNA, 21 nt, Homo sapiens
aucacauugccagggauuucc

SEQ ID NO: 1397 - RNA, 21 nt, Homo sapiens
aucacauugccagggauuacc

SEQ ID NO: 1398 - RNA, 22 nt, Homo sapiens
uggcucaguucagcaggaacag

SEQ ID NO: 1399 - RNA, 22 nt, Homo sapiens
cauugcacuugucucggucuga

SEQ ID NO: 1400 - RNA, 21 nt, Homo sapiens
uucaaguaauccaggauaggc

SEQ ID NO: 1401 - RNA, 22 nt, Homo sapiens
uucaaguaauucaggauagguu

SEQ ID NO: 1402 - RNA, 21 nt, Homo sapiens
uucacaguggcuaaguuccgc

SEQ ID NO: 1403 - RNA, 21 nt, Homo sapiens
uucacaguggcuaaguucugc

SEQ ID NO: 1404 - RNA, 22 nt, Homo sapiens
aaggagcucacagucuauugag

SEQ ID NO: 1405 - RNA, 21 nt, Homo sapiens
agggcccccccucaauccugu

SEQ ID NO: 1406 - RNA, 22 nt, Homo sapiens
uaugugggaugguaaaccgcuu

SEQ ID NO: 1407 - RNA, 22 nt, Homo sapiens
ugguuuaccgucccacauacau

SEQ ID NO: 1408 - RNA, 21 nt, Homo sapiens
uagcaccaucugaaaucgguu

SEQ ID NO: 1409 - RNA, 23 nt, Homo sapiens
uagcaccauuugaaaucaguguu

SEQ ID NO: 1410 - RNA, 20 nt, Homo sapiens
uagcaccauuugaaaucggu

SEQ ID NO: 1411 - RNA, 23 nt, Homo sapiens
cagugcaauaguauugucaaagc

SEQ ID NO: 1412 - RNA, 22 nt, Homo sapiens
uaaacguggauguacuugcuuu

SEQ ID NO: 1413 - RNA, 23 nt, Homo sapiens
acuuuaacauggaagugcuuucu

SEQ ID NO: 1414 - RNA, 22 nt, Homo sapiens
uuuaacauggggguaccugcug

SEQ ID NO: 1415 - RNA, 23 nt, Homo sapiens
uaagugcuuccauguuuugguga

SEQ ID NO: 1416 - RNA, 23 nt, Homo sapiens
uaagugcuuccauguuuuaguag

SEQ ID NO: 1417 - RNA, 23 nt, Homo sapiens
uaagugcuuccauguuucagugg

SEQ ID NO: 1418 - RNA, 23 nt, Homo sapiens
uaagugcuuccauguuugagugu

SEQ ID NO: 1419 - RNA, 22 nt, Homo sapiens
cuuucagucggauguuugcagc

SEQ ID NO: 1420 - RNA, 22 nt, Homo sapiens
uguaaacauccucgacuggaag

SEQ ID NO: 1421 - RNA, 22 nt, Homo sapiens
uguaaacauccuacacucagcu

SEQ ID NO: 1422 - RNA, 23 nt, Homo sapiens
uguaaacauccuacacucucagc

SEQ ID NO: 1423 - RNA, 22 nt, Homo sapiens
uguaaacauccccgacuggaag

SEQ ID NO: 1424 - RNA, 22 nt, Homo sapiens
cuuucagucggauguuuacagc

SEQ ID NO: 1425 - RNA, 20 nt, Homo sapiens
uguaaacauccuugacugga

SEQ ID NO: 1426 - RNA, 21 nt, Homo sapiens
ggcaagaugcuggcauagcug

SEQ ID NO: 1427 - RNA, 21 nt, Homo sapiens
uauugcacauuacuaaguugc

SEQ ID NO: 1428 - RNA, 23 nt, Homo sapiens
aaaagcuggguugagagggcgaa

SEQ ID NO: 1429 - RNA, 22 nt, Homo sapiens
gcacauuacacggucgaccucu

SEQ ID NO: 1430 - RNA, 22 nt, Homo sapiens
ccacugccccaggugcugcugg

SEQ ID NO: 1431 - RNA, 23 nt, Homo sapiens
cgcauccccuagggcauuggugu

SEQ ID NO: 1432 - RNA, 23 nt, Homo sapiens
ccuaguagguguccaguaagugu

SEQ ID NO: 1433 - RNA, 20 nt, Homo sapiens
ccucugggcccuuccuccag

SEQ ID NO: 1434 - RNA, 22 nt, Homo sapiens
cuggcccucucugcccuuccgu

SEQ ID NO: 1435 - RNA, 22 nt, Homo sapiens
aacacaccugguuaaccucuuu

SEQ ID NO: 1436 - RNA, 19 nt, Homo sapiens
gugcauuguaguugcauug

SEQ ID NO: 1437 - RNA, 23 nt, Homo sapiens
gcaaagcacacggccugcagaga

SEQ ID NO: 1438 - RNA, 21 nt, Homo sapiens
gccccugggccuauccuagaa

SEQ ID NO: 1439 - RNA, 23 nt, Homo sapiens
ucaagagcaauaacgaaaaaugu

SEQ ID NO: 1440 - RNA, 23 nt, Homo sapiens
uccagcuccuauaugaugccuuu

SEQ ID NO: 1441 - RNA, 23 nt, Homo sapiens
uccagcaucagugauuuuguuga

SEQ ID NO: 1442 - RNA, 21 nt, Homo sapiens
ucccuguccuccaggagcuca

SEQ ID NO: 1443 - RNA, 23 nt, Homo sapiens
uccgucucaguuacuuuauagcc

SEQ ID NO: 1444 - RNA, 24 nt, Homo sapiens
ucucacacagaaaucgcacccguc

SEQ ID NO: 1445 - RNA, 21 nt, Homo sapiens
ugcugacuccuaguccagggc

SEQ ID NO: 1446 - RNA, 23 nt, Homo sapiens
ugucugcccgcaugccugccucu

SEQ ID NO: 1447 - RNA, 23 nt, Homo sapiens
uggcagugucuuagcugguuguu

SEQ ID NO: 1448 - RNA, 23 nt, Homo sapiens
uaggcagugucauuagcugauug

SEQ ID NO: 1449 - RNA, 23 nt, Homo sapiens
aggcaguguaguuagcugauugc

SEQ ID NO: 1450 - RNA, 22 nt, Homo sapiens
uuaucagaaucuccagggguac

SEQ ID NO: 1451 - RNA, 23 nt, Homo sapiens
aauccuuggaaccuaggugugag

SEQ ID NO: 1452 - RNA, 22 nt, Homo sapiens
auugcacgguauccaucuguaa

SEQ ID NO: 1453 - RNA, 22 nt, Homo sapiens
uaaugccccuaaaaauccuuau

SEQ ID NO: 1454 - RNA, 22 nt, Homo sapiens
aauugcacuuuagcaaugguga

SEQ ID NO: 1455 - RNA, 22 nt, Homo sapiens
acauagaggaaauuccacguuu

SEQ ID NO: 1456 - RNA, 21 nt, Homo sapiens
aauaauacaugguugaucuuu

SEQ ID NO: 1457 - RNA, 22 nt, Homo sapiens
agaucgaccguguuauauucgc

SEQ ID NO: 1458 - RNA, 21 nt, Homo sapiens
gccugcugggguggaaccugg

SEQ ID NO: 1459 - RNA, 21 nt, Homo sapiens
gugccgccaucuuuugagugu

SEQ ID NO: 1460 - RNA, 23 nt, Homo sapiens
aaagugcugcgacauuugagcgu

SEQ ID NO: 1461 - RNA, 23 nt, Homo sapiens
gaagugcuucgauuuuggggugu

SEQ ID NO: 1462 - RNA, 22 nt, Homo sapiens
acucaaaaugggggcgcuuucc

SEQ ID NO: 1463 - RNA, 22 nt, Homo sapiens
uuauaauacaaccugauaagug

SEQ ID NO: 1464 - RNA, 22 nt, Homo sapiens
uuuguucguucggcucgcguga

SEQ ID NO: 1465 - RNA, 21 nt, Homo sapiens
aucauagaggaaaauccacgu

SEQ ID NO: 1466 - RNA, 22 nt, Homo sapiens
aucauagaggaaaauccauguu

SEQ ID NO: 1467 - RNA, 22 nt, Homo sapiens
aucacacaaaggcaacuuuugu

SEQ ID NO: 1468 - RNA, 22 nt, Homo sapiens
cuccugacuccagguccugugu

SEQ ID NO: 1469 - RNA, 19 nt, Homo sapiens
ugguagacuauggaacgua

SEQ ID NO: 1470 - RNA, 22 nt, Homo sapiens
uauguaauaugguccacaucuu

SEQ ID NO: 1471 - RNA, 22 nt, Homo sapiens
ugguugaccauagaacaugcgc

SEQ ID NO: 1472 - RNA, 22 nt, Homo sapiens
uauacaagggcaagcucucugu

SEQ ID NO: 1473 - RNA, 22 nt, Homo sapiens
gaaguuguucgugguggauucg

SEQ ID NO: 1474 - RNA, 22 nt, Homo sapiens
agaucagaaggugauuguggcu

SEQ ID NO: 1475 - RNA, 20 nt, Homo sapiens
auuccuagaaauuguucaua

SEQ ID NO: 1476 - RNA, 23 nt, Homo sapiens
cgaauguugcucggugaaccccu

SEQ ID NO: 1477 - RNA, 22 nt, Homo sapiens
agguuacccgagcaacuuugca

SEQ ID NO: 1478 - RNA, 22 nt, Homo sapiens
aauauaacacagauggccuguu

SEQ ID NO: 1479 - RNA, 23 nt, Homo sapiens
acuucaccugguccacuagccgu

SEQ ID NO: 1480 - RNA, 22 nt, Homo sapiens
cuggacuuagggucagaaggcc

SEQ ID NO: 1481 - RNA, 22 nt, Homo sapiens
cuggacuuggagucagaaggcc

SEQ ID NO: 1482 - RNA, 22 nt, Homo sapiens
agcucggucugaggccccucag

SEQ ID NO: 1483 - RNA, 22 nt, Homo sapiens
cagcagcaauucauguuuugaa

SEQ ID NO: 1484 - RNA, 21 nt, Homo sapiens
aucgggaaugucguguccgcc

SEQ ID NO: 1485 - RNA, 22 nt, Homo sapiens
uaauacugucugguaaaaccgu

SEQ ID NO: 1486 - RNA, 21 nt, Homo sapiens
ugucuugcaggccgucaugca

SEQ ID NO: 1487 - RNA, 23 nt, Homo sapiens
ucuuggaguaggucauugggugg

SEQ ID NO: 1488 - RNA, 21 nt, Homo sapiens
cuggauggcuccuccaugucu

SEQ ID NO: 1489 - RNA, 22 nt, Homo sapiens
aucaugaugggcuccucggugu

SEQ ID NO: 1490 - RNA, 22 nt, Homo sapiens
uugcauauguaggaugucccau

SEQ ID NO: 1491 - RNA, 22 nt, Homo sapiens
uggcaguguauuguuagcuggu

SEQ ID NO: 1492 - RNA, 22 nt, Homo sapiens
uuuuugcgauguguuccuaaua

SEQ ID NO: 1493 - RNA, 23 nt, Homo sapiens
aaaccguuaccauuacugaguuu

SEQ ID NO: 1494 - RNA, 22 nt, Homo sapiens
uguuugcagaggaaacugagac

SEQ ID NO: 1495 - RNA, 21 nt, Homo sapiens
ucagucucaucugcaaagaag

SEQ ID NO: 1496 - RNA, 22 nt, Homo sapiens
gagguuguccguggugaguucg

SEQ ID NO: 1497 - RNA, 20 nt, Homo sapiens
gucauacacggcucuccucu

SEQ ID NO: 1498 - RNA, 22 nt, Homo sapiens
agaggcuggccgugaugaauuc

SEQ ID NO: 1499 - RNA, 21 nt, Homo sapiens
cccagauaauggcacucucaa

SEQ ID NO: 1500 - RNA, 23 nt, Homo sapiens
agugacaucacauauacggcagc

SEQ ID NO: 1501 - RNA, 22 nt, Homo sapiens
caaccuggaggacuccaugcug

SEQ ID NO: 1502 - RNA, 23 nt, Homo sapiens
aguggggaacccuuccaugagga

SEQ ID NO: 1503 - RNA, 23 nt, Homo sapiens
aggaccugcgggacaagauucuu

SEQ ID NO: 1504 - RNA, 22 nt, Homo sapiens
uuguacaugguaggcuuucauu

SEQ ID NO: 1505 - RNA, 24 nt, Homo sapiens
ugaaacauacacgggaaaccucuu

SEQ ID NO: 1506 - RNA, 23 nt, Homo sapiens
aaacaaacauggugcacuucuuu

SEQ ID NO: 1507 - RNA, 17 nt, Homo sapiens
auuacauggccaaucuc

SEQ ID NO: 1508 - RNA, 21 nt, Homo sapiens
cagcagcacacugugguuugu

SEQ ID NO: 1509 - RNA, 23 nt, Homo sapiens
uuucaagccagggggcguuuuuc

SEQ ID NO: 1510 - RNA, 23 nt, Homo sapiens
uuaagacuugcagugauguuuaa

SEQ ID NO: 1511 - RNA, 22 nt, Homo sapiens
augcaccugggcaaggauucug

SEQ ID NO: 1512 - RNA, 22 nt, Homo sapiens
aauccuuugucccugggugaga

SEQ ID NO: 1513 - RNA, 21 nt, Homo sapiens
auccuugcuaucugggugcua

SEQ ID NO: 1514 - RNA, 23 nt, Homo sapiens
uagcagcgggaacaguucugcag

SEQ ID NO: 1515 - RNA, 21 nt, Homo sapiens
agacccuggucugcacucuau

SEQ ID NO: 1516 - RNA, 22 nt, Homo sapiens
gucaacacuugcugguuuccuc

SEQ ID NO: 1517 - RNA, 21 nt, Homo sapiens
uaaggcacccuucugaguaga

SEQ ID NO: 1518 - RNA, 21 nt, Homo sapiens
uuuugcaccuuuuggagugaa

SEQ ID NO: 1519 - RNA, 23 nt, Homo sapiens
ugauuguagccuuuuggaguaga

SEQ ID NO: 1520 - RNA, 23 nt, Homo sapiens
ugauugguacgucuguggguaga

SEQ ID NO: 1521 - RNA, 23 nt, Homo sapiens
uacucaggagaguggcaaucaca

SEQ ID NO: 1522 - RNA, 21 nt, Homo sapiens
gugucuuuugcucugcaguca

SEQ ID NO: 1523 - RNA, 22 nt, Homo sapiens
aagugcugucauagcugagguc

SEQ ID NO: 1524 - RNA, 23 nt, Homo sapiens
cacucagccuugagggcacuuuc

SEQ ID NO: 1525 - RNA, 22 nt, Homo sapiens
uucacagggaggugucauuuau

SEQ ID NO: 1526 - RNA, 20 nt, Homo sapiens
auugacacuucugugaguag

SEQ ID NO: 1527 - RNA, 21 nt, Homo sapiens
gagugccuucuuuuggagcgu

SEQ ID NO: 1528 - RNA, 24 nt, Homo sapiens
uucuccaaaagaaagcacuuucug

SEQ ID NO: 1529 - RNA, 18 nt, Homo sapiens
ugcuuccuuucagagggu

SEQ ID NO: 1530 - RNA, 22 nt, Homo sapiens
aucuggagguaagaagcacuuu

SEQ ID NO: 1531 - RNA, 22 nt, Homo sapiens
ccucuagauggaagcacugucu

SEQ ID NO: 1532 - RNA, 23 nt, Homo sapiens
aucgugcaucccuuuagaguguu

SEQ ID NO: 1533 - RNA, 22 nt, Homo sapiens
ucgugcaucccuuuagaguguu

SEQ ID NO: 1534 - RNA, 22 nt, Homo sapiens
aucgugcauccuuuuagagugu

SEQ ID NO: 1535 - RNA, 20 nt, Homo sapiens
ucugcaaagggaagcccuuu

SEQ ID NO: 1536 - RNA, 23 nt, Homo sapiens
ucucuggagggaagcacuuucug

SEQ ID NO: 1537 - RNA, 23 nt, Homo sapiens
cucuagagggaagcacuuucucu

SEQ ID NO: 1538 - RNA, 21 nt, Homo sapiens
aaagcgcuucccuuugcugga

SEQ ID NO: 1539 - RNA, 22 nt, Homo sapiens
caaagcgcuccccuuuagaggu

SEQ ID NO: 1540 - RNA, 22 nt, Homo sapiens
caaagcgcuucucuuuagagug

SEQ ID NO: 1541 - RNA, 21 nt, Homo sapiens
caaagcgcuucccuuuggagc

SEQ ID NO: 1542 - RNA, 22 nt, Homo sapiens
aaagcgcuucccuucagagugu

SEQ ID NO: 1543 - RNA, 21 nt, Homo sapiens
aaagcgcuucucuuuagagga

SEQ ID NO: 1544 - RNA, 22 nt, Homo sapiens
uucuccaaaagggagcacuuuc

SEQ ID NO: 1545 - RNA, 25 nt, Homo sapiens
aaagugcauccuuuuagaguguuac

SEQ ID NO: 1546 - RNA, 23 nt, Homo sapiens
aaagugcauccuuuuagagguuu

SEQ ID NO: 1547 - RNA, 22 nt, Homo sapiens
aaagugcaucuuuuuagaggau

SEQ ID NO: 1548 - RNA, 23 nt, Homo sapiens
caaagugccucccuuuagagugu

SEQ ID NO: 1549 - RNA, 22 nt, Homo sapiens
aaagugccuccuuuuagagugu

SEQ ID NO: 1550 - RNA, 21 nt, Homo sapiens
cuccagagggaaguacuuucu

SEQ ID NO: 1551 - RNA, 23 nt, Homo sapiens
ucuacaaagggaagcccuuucug

SEQ ID NO: 1552 - RNA, 22 nt, Homo sapiens
aaagugcuucccuuuggacugu

SEQ ID NO: 1553 - RNA, 21 nt, Homo sapiens
aaagugcuuccuuuuagaggg

SEQ ID NO: 1554 - RNA, 23 nt, Homo sapiens
aaagugcuuccuuuuagaggguu

SEQ ID NO: 1555 - RNA, 23 nt, Homo sapiens
aaagugcuucucuuugguggguu

SEQ ID NO: 1556 - RNA, 21 nt, Homo sapiens
aaagugcuuccuuuuugaggg

SEQ ID NO: 1557 - RNA, 22 nt, Homo sapiens
aagugcuuccuuuuagaggguu

SEQ ID NO: 1558 - RNA, 24 nt, Homo sapiens
acaaagugcuucccuuuagagugu

SEQ ID NO: 1559 - RNA, 22 nt, Homo sapiens
acaaagugcuucccuuuagagu

SEQ ID NO: 1560 - RNA, 22 nt, Homo sapiens
aacgcacuucccuuuagagugu

SEQ ID NO: 1561 - RNA, 23 nt, Homo sapiens
aaaaugguucccuuuagaguguu

SEQ ID NO: 1562 - RNA, 21 nt, Homo sapiens
aacgcgcuucccuauagaggg

SEQ ID NO: 1563 - RNA, 21 nt, Homo sapiens
gaaggcgcuucccuuuggagu

SEQ ID NO: 1564 - RNA, 22 nt, Homo sapiens
cuacaaagggaagcacuuucuc

SEQ ID NO: 1565 - RNA, 21 nt, Homo sapiens
cuccagagggaugcacuuucu

SEQ ID NO: 1566 - RNA, 21 nt, Homo sapiens
gaaggcgcuucccuuuagagc

SEQ ID NO: 1567 - RNA, 21 nt, Homo sapiens
aaagugcuuccuuuuagaggc

SEQ ID NO: 1568 - RNA, 21 nt, Homo sapiens
cucuagagggaagcacuuucu

SEQ ID NO: 1569 - RNA, 24 nt, Homo sapiens
cucuugagggaagcacuuucuguu

SEQ ID NO: 1570 - RNA, 24 nt, Homo sapiens
cucuagagggaagcgcuuucuguu

SEQ ID NO: 1571 - RNA, 21 nt, Homo sapiens
cugcaaagggaagcccuuucu

SEQ ID NO: 1572 - RNA, 22 nt, Homo sapiens
uggaagacuagugauuuuguug

SEQ ID NO: 1573 - RNA, 23 nt, Homo sapiens
ucuuugguuaucuagcuguauga

SEQ ID NO: 1574 - RNA, 21 nt, Homo sapiens
uaaagcuagauaaccgaaagu

SEQ ID NO: 1575 - RNA, 21 nt, Homo sapiens
uauugcacuugucccggccug

SEQ ID NO: 1576 - RNA, 22 nt, Homo sapiens
aaagugcuguucgugcagguag

SEQ ID NO: 1577 - RNA, 22 nt, Homo sapiens
uucaacggguauuuauugagca

SEQ ID NO: 1578 - RNA, 22 nt, Homo sapiens
uuuggcacuagcacauuuuugc

SEQ ID NO: 1579 - RNA, 22 nt, Homo sapiens
ugagguaguaaguuguauuguu

SEQ ID NO: 1580 - RNA, 22 nt, Homo sapiens
aacccguagauccgaucuugug

SEQ ID NO: 1581 - RNA, 22 nt, Homo sapiens
cacccguagaaccgaccuugcg